(19) United States
(12) Patent Application Publication
(10) Pub. No.: US 2023/0233561 A1
(43) Pub. Date: Jul. 27, 2023

(54) ASSESSING AND TREATING BIOLOGICAL AGING

(71) Applicant: Mayo Foundation for Medical Education and Research, Rochester, MN (US)

(72) Inventors: Nathan K. LeBrasseur, Rochester, MN (US); Marissa Joy Schafer, Rochester, MN (US); Thomas A. White, Spring Valley, MN (US); Elizabeth J. Atkinson, Rochester, MN (US); Xu Zhang, Rochester, MN (US)

(21) Appl. No.: 18/008,758

(22) PCT Filed: Jun. 17, 2021
(86) PCT No.: PCT/US2021/037816

§ 371 Date: Dec. 7, 2022

Related U.S. Application Data
(60) Provisional application No. 63/040,502, filed on Jun. 17, 2020.

(51) Int. Cl. A61K 31/506; A61K 31/352; A61K 31/635; A61K 31/395; A61K 31/4045; A61K 31/519; A61K 31/155; A61K 31/436; A61K 38/17; A61K 31/496; A61P 39/00; G01N 33/68

(52) U.S. Cl. CPC A61K 31/506; A61K 31/155; A61K 31/352; A61K 31/395; A61K 31/436; A61K 31/496; A61K 31/519; A61K 31/635; A61K 31/4045; A61K 38/1709; A61P 39/00; G01N 33/68; G01N 2800/52

(57) ABSTRACT

Abstract: This document relates to methods and materials for assessing biological aging. For example, methods and materials that can be used to determine if a mammal (e.g., a human) has an advanced biological age, is at risk of developing one or more adverse outcomes (e.g., adverse outcomes associated with medical intervention at an advanced biological age) following a medical intervention, and/or is likely to be responsive to one or more senotherapeutic agents are provided herein. In some cases, methods and materials for using one or more senotherapeutic agents to improve one or more outcomes for a mammal following a medical intervention (e.g., surgery) are also provided.

### CROSS-REFERENCE TO RELATED APPLICATIONS

This application claims the benefit of U.S. Pat. Application Serial No. 63/040,502, filed on Jun. 17, 2020. The disclosure of the prior application is considered part of (and is incorporated by reference in) the disclosure of this application.

### STATEMENT REGARDING FEDERAL FUNDING

This invention was made with government support under AG055529 and AG052958 awarded by the National Institutes of Health. The government has certain rights in the invention.

### TECHNICAL FIELD

This document relates to methods and materials for assessing biological aging. For example, methods and materials provided herein can be used to determine if a mammal (e.g., a human) has an advanced biological age, is at risk of developing one or more adverse outcomes (e.g., adverse outcomes associated with medical intervention at an advanced biological age) following a medical intervention, and/or is likely to be responsive to one or more senotherapeutic agents. In some cases, this document provides methods and materials for using one or more senotherapeutic agents to improve one or more outcomes for a mammal following a medical intervention (e.g., surgery).

### BACKGROUND INFORMATION

Aging is the strongest risk factor for the majority of chronic diseases. Cellular senescence, a state of stable growth arrest caused by diverse forms of cellular and molecular damage, contributes to aging, and senescent cells accumulate with advancing age. Preclinical studies in rodents have established that both transgenic strategies and drugs that selectively kill senescent cells improve numerous yet pathologically distinct conditions of aging, including idiopathic pulmonary fibrosis (Schafer et al., Nat Commun.; 8:14532 (2017)), cardiovascular disease (Roos et al., Aging Cell.; 15(5):973-7 (2016); and Childs et al., Science.; 354(6311):472-7 (2016)), hepatic steatosis (Ogrodnik et al., Nat Commun.; 8:15691 (2017)), osteoporosis (Farr et al., Nat Med.; 23(9):1072-9 (2017)), diabetes (Palmer et al., Aging Cell.; 18(3):e12950 (2019)), physical decline (Xu et al., Nat Med.; 24(8):1246-56 (2018); and Baker et al., Nature.; 530(7589):184-9 (2016)), and brain dysfunction (Musi et al., Aging Cell.; 17(6):e12840 (2018); Bussian et al., Nature.; 562(7728):578-82 (2018); and Ogrodnik et al., Cell Metab.; 29(5):1061-77 e8 (2019)).

### SUMMARY

Dramatic variability is inherent to aging, with many older adults of a given chronological age experiencing multiple chronic conditions and functional limitations, while paired-age counterparts may have low or no disease burden and comparatively greater functional independence. Individuals with cumulatively more age-related impairments may be characterized as frail or biologically older according to a standardized accumulation of deficits index (Searle et al., BMC Geriatr.; 8:24 (2008)).

This document provides methods and materials related to assessing biological aging. In some cases, this document provides methods and materials for identifying a mammal (e.g., a human) as having an advanced biological age, as being at risk of developing one or more adverse outcomes (e.g., adverse outcomes associated with medical intervention at an advanced biological age) following a medical intervention, and/or as being likely to be responsive to one or more senotherapeutic agents. For example, this document provides methods and materials for detecting the presence or absence of an elevated level of expression of one or more polypeptides secreted by senescent cells (e.g., one or more senescence-associated secretory phenotype (SASP) polypeptides) within a mammal (e.g., a human) and classifying the mammal as having an advanced biological age, as being at risk of developing one or more adverse outcomes following a medical intervention, and/or as being likely to be responsive to one or more senotherapeutic agents if the presence of an elevated level of expression of one or more (e.g., four, five, six, or seven) SASP polypeptides is detected. In some cases, this document provides methods and materials for treating a mammal (e.g., a mammal identified as having an advanced biological age, as being at risk of developing one or more adverse outcomes following a medical intervention, and/or as being likely to be responsive to one or more senotherapeutic agents) undergoing one or more medical interventions (e.g., surgery or chemotherapy) to improve outcomes for that mammal following the medical intervention(s). For example, one or more senotherapeutic agents can be administered to a mammal (e.g., a human) identified as having an advanced biological age, as being at risk of developing one or more adverse outcomes following a medical intervention, and/or as being likely to be responsive to one or more senotherapeutic agents to reduce the risk of that mammal developing one or more adverse outcomes.

As demonstrated herein, circulating concentrations of select SASP polypeptides positively associate with age, frailty, and adverse post-surgery outcomes. For example, a panel that includes four, five, six, or seven of the following SASP factors can be used to identify an advanced biological age and can predict risk for adverse outcomes (e.g., surgical complications, ICU admission, rehospitalization, and/or mortality) in older adults in response to surgical intervention(s): (1) growth differentiation factor 15 (GDF15), (2) TNF Receptor Superfamily Member 6 (FAS), (3) osteopontin (OPN), (4) tumor necrosis factor receptor 1 (TNFR1), (5) ACTIVIN A, (6) chemokine (C-C motif) ligand 3 (CCL3), and (7) interleukin 15 (IL15).

Having the ability to identify a mammal (e.g., a human) as having an advanced biological age, as being at risk of developing one or more adverse outcomes (e.g., adverse outcomes associated with medical intervention at an advanced biological age) following a medical intervention, and/or as being likely to be responsive to one or more senotherapeutic agents provides a unique and unrealized opportunity to evaluate age-related health and to improve surgical outcomes by reducing morbidity and mortality. For example, an elevated level of expression of one or more (e.g., four, five, six, or seven) SASP polypeptides can be used to identify a mammal (e.g., a human) having an advanced biological age, as being at risk of developing one or more adverse outcomes following a medical intervention, and/or as being likely to be responsive to one or more senotherapeutic agents and guide clinical decision making. For example, an elevated level of expression of one or more (e.g., four, five, six, or seven) SASP polypeptides can be used to identify humans who may be most responsive to emerging therapies and can serve as an endpoint in associated clinical trials. Accordingly, the methods and materials provided herein have wide relevance for clinical practice and clinical research.

In general, one aspect of this document features methods for assessing the biological age of a mammal. The methods can include, or consist essentially of, (a) obtaining the chronological age of a mammal, (b) obtaining a reference level of expression for each of four or more SASP polypeptides for the chronological age of the mammal; (c) detecting the presence or absence of an elevated level of expression level of the SASP polypeptides in a sample obtained from the mammal as compared to the reference level, (d) classifying the mammal as having an advanced biological age based at least in part on the presence of the elevated levels, and (e) classifying the mammal as not having the advanced biological age based at least in part on the absence of the elevated levels. The mammal can be a human. The SASP polypeptides can be selected from the group consisting of a GDF15 polypeptide, a FAS polypeptide, an OPN polypeptide, a TNFR1 polypeptide, an ACTIVIN A polypeptide, a CCL3 polypeptide, an IL15 polypeptide, and combinations thereof. The SASP polypeptides can include a GDF15 polypeptide, a FAS polypeptide, an OPN polypeptide, a TNFR1 polypeptide, an ACTIVIN A polypeptide, a CCL3 polypeptide, and an IL15 polypeptide. The sample can be whole blood, serum, plasma, urine, cerebrospinal fluid, skeletal muscle tissue, adipose tissue, kidney tissue, bone tissue, or liver tissue.

In some cases, the aspect in the previous paragraph can include, for step (b), obtaining a reference level of expression for an IL15 polypeptide and obtaining a reference level of expression for three or more (e.g., three, four, five, or six) of the following SASP polypeptides: a GDF15 polypeptide, a FAS polypeptide, an OPN polypeptide, a TNFR1 polypeptide, an ACTIVIN A polypeptide, a CCL3 polypeptide, and can include, for step (c), detecting the presence or absence of an elevated level of expression of the IL 15 polypeptide and detecting the presence or absence of an elevated level of expression of the three or more (e.g., three, four, five, or six) SASP polypeptides.In another aspect, this document features methods for identifying a mammal as being at risk of developing an adverse outcome following a medical intervention. The methods can include, or consist essentially of, (a) obtaining the chronological age of a mammal, (b) obtaining a reference level of expression for each of four or more SASP polypeptides for the chronological age of the mammal; (c) detecting the presence or absence of an elevated level of expression level of the SASP polypeptides in a sample obtained from the mammal as compared to the reference level, (d) classifying the mammal as being at risk of developing the adverse outcome following the medical intervention based at least in part on the presence of the elevated levels, and (e) classifying said mammal as not being at risk of developing the adverse outcome following the medical intervention based at least in part on the absence of the elevated levels. The mammal can be a human. The SASP polypeptides can be selected from the group consisting of a GDF15 polypeptide, a FAS polypeptide, an OPN polypeptide, a TNFR1 polypeptide, an ACTIVIN A polypeptide, a CCL3 polypeptide, an IL15 polypeptide, and combinations thereof. The SASP polypeptides can include a GDF15 polypeptide, a FAS polypeptide, an OPN polypeptide, a TNFR1 polypeptide, an ACTIVIN A polypeptide, a CCL3 polypeptide, and an IL15 polypeptide. The sample can be whole blood, serum, plasma, urine, cerebrospinal fluid, skeletal muscle tissue, adipose tissue, kidney tissue, bone tissue, or liver tissue.

In some cases, the aspect in the previous paragraph can include, for step (b), obtaining a reference level of expression for an IL15 polypeptide and obtaining a reference level of expression for three or more (e.g., three, four, five, or six) of the following SASP polypeptides: a GDF15 polypeptide, a FAS polypeptide, an OPN polypeptide, a TNFR1 polypeptide, an ACTIVIN A polypeptide, a CCL3 polypeptide, and can include, for step (c), detecting the presence or absence of an elevated level of expression of the IL 15 polypeptide and detecting the presence or absence of an elevated level of expression of the three or more (e.g., three, four, five, or six) SASP polypeptides.

In another aspect, this document features methods for identifying a mammal as likely to be responsive to a senotherapeutic agent. The methods can include, or consist essentially of, (a) obtaining the chronological age of a mammal, (b) obtaining a reference level of expression for each of four or more SASP polypeptides for the chronological age of the mammal; (c) detecting the presence or absence of an elevated level of expression level of the SASP polypeptides in a sample obtained from the mammal as compared to the reference level, (d) classifying the mammal as being likely to be responsive to the senotherapeutic agent based at least in part on the presence of the elevated levels, and (e) classifying the mammal as not being likely to be responsive to the senotherapeutic agent based at least in part on the absence of the elevated levels. The mammal can be a human. The SASP polypeptides can be selected from the group consisting of a GDF15 polypeptide, a FAS polypeptide, an OPN polypeptide, a TNFR1 polypeptide, an ACTIVIN A polypeptide, a CCL3 polypeptide, an IL15 polypeptide, and combinations thereof. The SASP polypeptides can include a GDF15 polypeptide, a FAS polypeptide, an OPN polypeptide, a TNFR1 polypeptide, an ACTIVIN A polypeptide, a CCL3 polypeptide, and an IL15 polypeptide. The sample can be whole blood, serum, plasma, urine, cerebrospinal fluid, skeletal muscle tissue, adipose tissue, kidney tissue, bone tissue, or liver tissue.

In some cases, the aspect in the previous paragraph can include, for step (b), obtaining a reference level of expression for an IL15 polypeptide and obtaining a reference level of expression for three or more (e.g., three, four, five, or six) of the following SASP polypeptides: a GDF15 polypeptide, a FAS polypeptide, an OPN polypeptide, a TNFR1 polypeptide, an ACTIVIN A polypeptide, a CCL3 polypeptide, and can include, for step (c), detecting the presence or absence of an elevated level of expression of the IL 15 polypeptide and detecting the presence or absence of an elevated level of expression of the three or more (e.g., three, four, five, or six) SASP polypeptides.

In another aspect, this document features methods for identifying a mammal as having an enriched systemic senescent cell burden. The methods can include, or consist essentially of, (a) obtaining the chronological age of a mammal, (b) obtaining a reference level of expression for each of four or more SASP polypeptide for the chronological age of the mammal; (c) detecting the presence or absence of an elevated level of expression level of the SASP polypeptides in a sample obtained from the mammal as compared to the reference level, (d) classifying the mammal as having the enriched systemic senescent cell burden based at least in part on the presence of the elevated levels, and (e) classifying the mammal as not having the enriched systemic senescent cell burden based at least in part on the absence of the elevated levels. The mammal can be a human. The SASP polypeptides can be selected from the group consisting of a GDF15 polypeptide, a FAS polypeptide, an OPN polypeptide, a TNFR1 polypeptide, an ACTIVIN A polypeptide, a CCL3 polypeptide, an IL15 polypeptide, and combinations thereof. The SASP polypeptides can include a GDF15 polypeptide, a FAS polypeptide, an OPN polypeptide, a TNFR1 polypeptide, an ACTIVIN A polypeptide, a CCL3 polypeptide, and an IL15 polypeptide. The sample can be whole blood, serum, plasma, urine, cerebrospinal fluid, skeletal muscle tissue, adipose tissue, kidney tissue, bone tissue, or liver tissue.

In some cases, the aspect in the previous paragraph can include, for step (b), obtaining a reference level of expression for an IL15 polypeptide and obtaining a reference level of expression for three or more (e.g., three, four, five, or six) of the following SASP polypeptides: a GDF15 polypeptide, a FAS polypeptide, an OPN polypeptide, a TNFR1 polypeptide, an ACTIVIN A polypeptide, a CCL3 polypeptide, and can include, for step (c), detecting the presence or absence of an elevated level of expression of the IL 15 polypeptide and detecting the presence or absence of an elevated level of expression of the three or more (e.g., three, four, five, or six) SASP polypeptides.

In another aspect, this document features methods for treating a mammal having frailty. The methods can include, or consist essentially of, (a) identifying a mammal as having an elevated level of expression for each of four or more SASP polypeptides, for the mammal’s chronological age, in a sample from the mammal; and (b) administering a senotherapeutic agent to the mammal. The can be a human. The senotherapeutic agent can be dasatinib, quercetin, navitoclax, A1331852, A1155463, fisetin, luteolin, geldanamycin, tanespimycin, alvespimycin, piperlongumine, panobinostat, FOX04-related peptides, nutlin3a, ruxolitinib, metformin, or rapamycin. The senotherapeutic agent can be effective to reduce or eliminate a symptom of frailty. The symptom of frailty can be unintentional weight loss, exhaustion, muscle weakness, slowness while walking, low levels of activity, inflammation, difficulties with activities of daily living, or combinations thereof.

In some cases, the aspect in the previous paragraph can include, for step (a), identifying the mammal as having an elevated level of expression of an IL15 polypeptide and as having an elevated level of expression of three or more (e.g., three, four, five, or six) of the following SASP polypeptides: a GDF15 polypeptide, a FAS polypeptide, an OPN polypeptide, a TNFR1 polypeptide, an ACTIVIN A polypeptide, a CCL3 polypeptide.

In another aspect, this document features methods for treating a mammal having frailty. The methods can include, or consist essentially of, administering a senotherapeutic agent to a mammal identified as having an elevated level of expression for each of four or more SASP polypeptides, for the mammal’s chronological age, in a sample from the mammal. The can be a human. The senotherapeutic agent can be dasatinib, quercetin, navitoclax, A1331852, A1155463, fisetin, luteolin, geldanamycin, tanespimycin, alvespimycin, piperlongumine, panobinostat, FOX04-related peptides, nutlin3a, ruxolitinib, metformin, or rapamycin. The senotherapeutic agent can be effective to reduce or eliminate a symptom of frailty. The symptom of frailty can be unintentional weight loss, exhaustion, muscle weakness, slowness while walking, low levels of activity, inflammation, difficulties with activities of daily living, or combinations thereof.

In some cases, the aspect in the previous paragraph can include administering a senotherapeutic agent to a mammal identified as having an elevated level of expression of an IL15 polypeptide and as having an elevated level of expression of three or more (e.g., three, four, five, or six) of the following SASP polypeptides: a GDF15 polypeptide, a FAS polypeptide, an OPN polypeptide, a TNFR1 polypeptide, an ACTIVIN A polypeptide, a CCL3 polypeptide.

In another aspect, this document features methods for improving the outcome of a mammal undergoing a medical intervention. The methods can include, or consist essentially of, (a) identifying a mammal as having an elevated level of expression for each of four or more SASP polypeptides, for the mammal’s chronological age, in a sample from the mammal; and (b) administering a senotherapeutic agent to the mammal. The mammal can be a human. The senotherapeutic agent can be dasatinib, quercetin, navitoclax, A1331852, A1155463, fisetin, luteolin, geldanamycin, tanespimycin, alvespimycin, piperlongumine, panobinostat, FOX04-related peptides, nutlin3a, ruxolitinib, metformin, or rapamycin. The senotherapeutic agent can be effective to reduce or eliminate an adverse event that can occur following a medical intervention. The adverse event can be myocardial infarction, new arrhythmia, new conduction abnormality, stroke, deep venous thrombosis, pulmonary emboli, pneumonia, plural effusion, new renal insufficiency, GI bleeding, new seizure disorder, significant hypotension, significant tachycardia, significant bradycardia, urinary tract infection, other infection, acute dementia, vascular complication, acute kidney injury, or combinations thereof. The medical intervention can include a surgery.

In some cases, the aspect in the previous paragraph can include, for step (a), identifying the mammal as having an elevated level of expression of an IL15 polypeptide and as having an elevated level of expression of three or more (e.g., three, four, five, or six) of the following SASP polypeptides: a GDF15 polypeptide, a FAS polypeptide, an OPN polypeptide, a TNFR1 polypeptide, an ACTIVIN A polypeptide, a CCL3 polypeptide.

In another aspect, this document features methods for improving the outcome of a mammal undergoing a medical intervention. The methods can include, or consist essentially of, administering a senotherapeutic agent to a mammal identified as having an elevated level of expression for each of four or more SASP polypeptides, for the mammal’s chronological age, in a sample from the mammal. The mammal can be a human. The senotherapeutic agent can be dasatinib, quercetin, navitoclax, A1331852, A1155463, fisetin, luteolin, geldanamycin, tanespimycin, alvespimycin, piperlongumine, panobinostat, FOX04-related peptides, nutlin3a, ruxolitinib, metformin, or rapamycin. The senotherapeutic agent can be effective to reduce or eliminate an adverse event that can occur following a medical intervention. The adverse event can be myocardial infarction, new arrhythmia, new conduction abnormality, stroke, deep venous thrombosis, pulmonary emboli, pneumonia, plural effusion, new renal insufficiency, GI bleeding, new seizure disorder, significant hypotension, significant tachycardia, significant bradycardia, urinary tract infection, other infection, acute dementia, vascular complication, acute kidney injury, or combinations thereof. The medical intervention can include a surgery.

In some cases, the aspect in the previous paragraph can include administering a senotherapeutic agent to a mammal identified as having an elevated level of expression of an IL15 polypeptide and as having an elevated level of expression of three or more (e.g., three, four, five, or six) of the following SASP polypeptides: a GDF15 polypeptide, a FAS polypeptide, an OPN polypeptide, a TNFR1 polypeptide, an ACTIVIN A polypeptide, a CCL3 polypeptide.

In another aspect, this document features methods for reducing a systemic senescent cell burden of a mammal. The methods can include, or consist essentially of, (a) identifying a mammal as having an elevated level of expression for each of four or more SASP polypeptides, for the mammal’s chronological age, in a sample from the mammal; and (b) administering a senotherapeutic agent to the mammal. The mammal can be a human. The senotherapeutic agent can be dasatinib, quercetin, navitoclax, A1331852, A1155463, fisetin, luteolin, geldanamycin, tanespimycin, alvespimycin, piperlongumine, panobinostat, FOX04-related peptides, nutlin3a, ruxolitinib, metformin, or rapamycin.

In some cases, the aspect in the previous paragraph can include, for step (a), identifying the mammal as having an elevated level of expression of an IL15 polypeptide and as having an elevated level of expression of three or more (e.g., three, four, five, or six) of the following SASP polypeptides: a GDF15 polypeptide, a FAS polypeptide, an OPN polypeptide, a TNFR1 polypeptide, an ACTIVIN A polypeptide, a CCL3 polypeptide.

In another aspect, this document features methods for reducing a systemic senescent cell burden a mammal. The methods can include, or consist essentially of, administering a senotherapeutic agent to a mammal identified as having an elevated level of expression for each of four or more SASP polypeptides, for the mammal’s chronological age, in a sample from the mammal. The mammal can be a human. The senotherapeutic agent can be dasatinib, quercetin, navitoclax, A1331852, A1155463, fisetin, luteolin, geldanamycin, tanespimycin, alvespimycin, piperlongumine, panobinostat, FOX04-related peptides, nutlin3a, ruxolitinib, metformin, or rapamycin.

In some cases, the aspect in the previous paragraph can include administering a senotherapeutic agent to a mammal identified as having an elevated level of expression of an IL15 polypeptide and as having an elevated level of expression of three or more (e.g., three, four, five, or six) of the following SASP polypeptides: a GDF15 polypeptide, a FAS polypeptide, an OPN polypeptide, a TNFR1 polypeptide, an ACTIVIN A polypeptide, a CCL3 polypeptide.

In another aspect, this document features methods for improving the outcome of a mammal undergoing a medical intervention. The methods can include, or consist essentially of, (a) identifying a mammal as having an elevated level of expression for each of four or more SASP polypeptides, for the mammal’s chronological age, in a sample from the mammal; and (b) selecting the mammal for more frequent monitoring following a medical intervention. The mammal can be a human. The adverse event can be myocardial infarction, new arrhythmia, new conduction abnormality, stroke, deep venous thrombosis, pulmonary emboli, pneumonia, plural effusion, new renal insufficiency, GI bleeding, new seizure disorder, significant hypotension, significant tachycardia, significant bradycardia, urinary tract infection, other infection, acute dementia, vascular complication, acute kidney injury, or combinations thereof. The medical intervention can include a surgery. The method can include performing the more frequent monitoring.

In some cases, the aspect in the previous paragraph can include, for step (a), identifying the mammal as having an elevated level of expression of an IL15 polypeptide and as having an elevated level of expression of three or more (e.g., three, four, five, or six) of the following SASP polypeptides: a GDF15 polypeptide, a FAS polypeptide, an OPN polypeptide, a TNFR1 polypeptide, an ACTIVIN A polypeptide, a CCL3 polypeptide.

In another aspect, this document features methods for improving the outcome of a mammal undergoing a medical intervention. The methods can include, or consist essentially of, selecting a mammal identified as having an elevated level of expression for each of four or more SASP polypeptides, for the mammal’s chronological age, in a sample from the mammal for more frequent monitoring following a medical intervention. The mammal can be a human. The adverse event can be myocardial infarction, new arrhythmia, new conduction abnormality, stroke, deep venous thrombosis, pulmonary emboli, pneumonia, plural effusion, new renal insufficiency, GI bleeding, new seizure disorder, significant hypotension, significant tachycardia, significant bradycardia, urinary tract infection, other infection, acute dementia, vascular complication, acute kidney injury, or combinations thereof. The medical intervention can include a surgery. The method can include performing the more frequent monitoring.

In some cases, the aspect in the previous paragraph can include selecting a mammal identified as having an elevated level of expression of an IL15 polypeptide and as having an elevated level of expression of three or more (e.g., three, four, five, or six) of the following SASP polypeptides: a GDF15 polypeptide, a FAS polypeptide, an OPN polypeptide, a TNFR1 polypeptide, an ACTIVIN A polypeptide, a CCL3 polypeptide, for more frequent monitoring following a medical intervention.

In another aspect, this document features methods for improving the outcome of a mammal undergoing a medical intervention. The methods can include, or consist essentially of, (a) identifying a mammal as having an elevated level of expression for each of four or more SASP polypeptides, for the mammal’s chronological age, in a sample from the mammal; and (b) selecting the mammal for more robust transitional care following a medical intervention. The mammal can be a human. The adverse event can be myocardial infarction, new arrhythmia, new conduction abnormality, stroke, deep venous thrombosis, pulmonary emboli, pneumonia, plural effusion, new renal insufficiency, GI bleeding, new seizure disorder, significant hypotension, significant tachycardia, significant bradycardia, urinary tract infection, other infection, acute dementia, vascular complication, acute kidney injury, or combinations thereof. The medical intervention can include a surgery. The method can include performing a more robust transitional care.

In some cases, the aspect in the previous paragraph can include, for step (a), identifying the mammal as having an elevated level of expression of an IL15 polypeptide and as having an elevated level of expression of three or more (e.g., three, four, five, or six) of the following SASP polypeptides: a GDF15 polypeptide, a FAS polypeptide, an OPN polypeptide, a TNFR1 polypeptide, an ACTIVIN A polypeptide, a CCL3 polypeptide.

In another aspect, this document features methods for improving the outcome of a mammal undergoing a medical intervention. The methods can include, or consist essentially of, selecting a mammal identified as having an elevated level of expression for each of four or more SASP polypeptides, for the mammal’s chronological age, in a sample from the mammal for more robust transitional care following a medical intervention. The mammal can be a human. The adverse event can be myocardial infarction, new arrhythmia, new conduction abnormality, stroke, deep venous thrombosis, pulmonary emboli, pneumonia, plural effusion, new renal insufficiency, GI bleeding, new seizure disorder, significant hypotension, significant tachycardia, significant bradycardia, urinary tract infection, other infection, acute dementia, vascular complication, acute kidney injury, or combinations thereof. The medical intervention can include a surgery. The method can include performing a more robust transitional care.

In some cases, the aspect in the previous paragraph can include selecting a mammal identified as having an elevated level of expression of an IL15 polypeptide and as having an elevated level of expression of three or more (e.g., three, four, five, or six) of the following SASP polypeptides: a GDF15 polypeptide, a FAS polypeptide, an OPN polypeptide, a TNFR1 polypeptide, an ACTIVIN A polypeptide, a CCL3 polypeptide, for more robust transitional care following a medical intervention.

In another aspect, this document features methods for improving the outcome of a mammal undergoing a medical intervention. The methods can include, or consist essentially of, (a) identifying a mammal as having an elevated level of expression for each of four or more SASP polypeptides, for the mammal’s chronological age, in a sample from the mammal; and (b) selecting the mammal to undergo a lifestyle intervention. The mammal can be a human. The lifestyle intervention can be a change in diet or increased exercise. The lifestyle intervention can include a change in diet and increased exercise. The adverse event can be myocardial infarction, new arrhythmia, new conduction abnormality, stroke, deep venous thrombosis, pulmonary emboli, pneumonia, plural effusion, new renal insufficiency, GI bleeding, new seizure disorder, significant hypotension, significant tachycardia, significant bradycardia, urinary tract infection, other infection, acute dementia, vascular complication, acute kidney injury, or combinations thereof.

In some cases, the aspect in the previous paragraph can include, for step (a), identifying the mammal as having an elevated level of expression of an IL15 polypeptide and as having an elevated level of expression of three or more (e.g., three, four, five, or six) of the following SASP polypeptides: a GDF15 polypeptide, a FAS polypeptide, an OPN polypeptide, a TNFR1 polypeptide, an ACTIVIN A polypeptide, a CCL3 polypeptide.

In another aspect, this document features methods for improving the outcome of a mammal undergoing a medical intervention. The methods can include, or consist essentially of, selecting a mammal identified as having an elevated level of expression for each of four or more SASP polypeptides, for the mammal’s chronological age, in a sample from the mammal to undergo a lifestyle intervention. The mammal can be a human. The lifestyle intervention can be a change in diet or increased exercise. The lifestyle intervention can include a change in diet and increased exercise. The adverse event can be myocardial infarction, new arrhythmia, new conduction abnormality, stroke, deep venous thrombosis, pulmonary emboli, pneumonia, plural effusion, new renal insufficiency, GI bleeding, new seizure disorder, significant hypotension, significant tachycardia, significant bradycardia, urinary tract infection, other infection, acute dementia, vascular complication, acute kidney injury, or combinations thereof.

In some cases, the aspect in the previous paragraph can include selecting a mammal identified as having an elevated level of expression of an IL15 polypeptide and as having an elevated level of expression of three or more (e.g., three, four, five, or six) of the following SASP polypeptides: a GDF15 polypeptide, a FAS polypeptide, an OPN polypeptide, a TNFR1 polypeptide, an ACTIVIN A polypeptide, a CCL3 polypeptide, to undergo a lifestyle intervention.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1C show that senescent human cells secrete a heterogeneous SASP. FIG. 1A. SA-β-Gal staining confirmed senescence induction in irradiated versus sham-treated human cells (scale bar 200 mm). FIG. 1B. Fold change in concentration of secreted SASP proteins by irradiated senescent cells (SnC) normalized to the sham control (C) samples for each cell type. FIG. 1C. Absolute secreted protein concentration (pg/mL) from one million senescent versus non-senescent control cells. (endothelial cells (endo), preadipocytes (pre), fibroblasts (fibro), epithelial cells (epi), and myoblasts (myo); mean depicted; two-tailed t-tests with significance indicated as p < 0.05*, 0.01 **, and 0.001***; n = 3 replicates per cell type). See Tables 2 and 3 for supportive data.

FIGS. 2A-2F show markers of senescence in irradiated cells. FIG. 2A. Percentage of endothelial cells (endo), preadipocytes (pre), fibroblasts (fibro), epithelial cells (epi), and myoblasts (myo) staining positively for SA-β-Gal. Expression of cyclin dependent kinase inhibitors and a SASP factor in (FIG. 2B) endo, (FIG. 2C) pre, (FIG. 2D) fibro, (FIG. 2E) epi, and (FIG. 2F) myo cells in culture. (Mean + SEM; individual two-tailed t-tests with significance indicated as p < 0.05*, 0.01**, and 0.001***; n = 3 replicates per cell type).

FIGS. 3A-3H show that circulating SASP factors are associated with chronological age. Circulating concentrations of SASP proteins GDF15 (FIG. 3A), ACTIVIN A (FIG. 3B), TNFR1 (FIG. 3C), CCL4 (FIG. 3D), FAS (FIG. 3E), CCL3 (FIG. 3F), TNFα (FIG. 3G), and IL6 (FIG. 3H) demonstrating the strongest unadjusted Spearman correlations with chronological age are depicted among biobank participants age 20-90 years. Women (n = 137) are indicated by pink circles, and men (n = 130) are indicated by blue circles. See Table 5 for supportive data.

FIGS. 4A-4H shows that circulating SASP factors are associated with increased risk of adverse post-operative outcomes. Levels of circulating SASP factors were compared among older adults who underwent surgery for severe aortic stenosis and (FIG. 4A) experienced at least one adverse event (n = 42) or were (FIG. 4B) rehospitalized within 12 months of hospital discharge (n = 28) (black circles) to counterparts who did not experience adverse outcomes (no adverse event, n = 55; no rehospitalization, n = 69) (gray circles). FIG. 4C. Among older women who underwent surgery for ovarian cancer, circulating SASP factors were compared for participants who were admitted to the ICU within 30 days of surgery (n = 11) (black triangles) versus those that were not (n = 22) (gray triangles). (FIGS. 4A-4C: median + 95% confidence interval are indicated with Kruskal-Wallis rank sum test results.) ROC AUCs indicating the adverse outcome risk discriminatory ability of a panel that includes seven SASP factors (GDF15, FAS, OPN, TNFR1, ACTIVIN A, CCL3, and IL15), a single top SASP factor (GDF15 or FAS), frailty score, or age + sex or age for older adults undergoing surgery for (FIGS. 4D-4E) severe aortic stenosis or (FIG. 4F) age for older adults undergoing surgery for ovarian cancer. FIG. 4G. All study participants in which accumulation of deficit frailty status was determined and all proteins were measured (n = 343) were clustered based on the presence of any post-surgical adverse event, frailty score, and chronological age. Six phenotypic clusters emerged: (1) non-frail, younger, and no adverse events (n = 32); (2) non-frail, older, and no adverse events (n = 25); (3) lower frailty score and no adverse events (n = 42); (4) intermediate frailty score and no adverse events (n = 82); (5) higher frailty score and adverse events (n = 53); (6) higher frailty score and no adverse events (n = 109). FIG. 4H. Scaled concentration comparison of the seven SASP proteins identified by GBM as associated with adverse events among the six clusters.

FIGS. 5A-5C show phenotypic cluster definition. All study participants in which accumulation of deficit frailty status was determined and all proteins were measured were clustered based on the presence of (FIG. 5A) any post-surgical adverse event, (FIG. 5B) frailty score, and (FIG. 5C) chronological age (n = 343).

FIG. 6 shows a ten-year survival curve of 224 women with ovarian cancer who underwent surgery. Participants were divided into four quartiles (Q1-Q4) based on age, BMI, and seven biomarkers of aging (GDF15, TNFR1, FAS, ACTIVIN A, IL-15, OPN, and MIP1A). Q1 represents participants with lowest values and Q4 represents participants with highest values.

FIG. 7 shows a correlation matrix of age, BMI, and seven biomarkers of aging (GDF15, TNFR1, FAS, ACTIVIN A, IL-15, OPN, and MIP1A) in 224 women with ovarian cancer.

FIG. 8 shows a ten-year survival curve of 224 women with ovarian cancer who underwent surgery. Participants were divided into four quartiles (Q1-Q4) based on age, BMI, and seven biomarkers of aging (GDF15, TNFR1, FAS, ACTIVIN A, IL-15, OPN, and MIP1A). Q1 represents participants with lowest values and Q4 represents participants with highest values.

FIGS. 9A - 9F. Predictive ability of post-surgical residual disease status (FIG. 9A), plasma concentrations of TNFR1 (FIG. 9B), FAS (FIG. 9C), and GDF15 (FIG. 9D), frailty index (FIG. 9E), and BMI (FIG. 9F) for survival in 224 women with ovarian cancer (negative predictive values reflect a beneficial influence on survival, positive values reflect a negative influence on survival).

FIG. 10 shows a ten-year survival curve of 224 women with ovarian cancer who underwent surgery. Participants were divided into four quartiles (Q1-Q4) based on the following clinical variables: age, BMI, ASA score, albumin, FIGO, serous histology, surgical complexity, residual disease, and ascites. Q1 represents participants with lowest/favorable values and Q4 represents participants with highest/unfavorable healthy values.

FIGS. 11A - 11D. In statistical models limited to clinical variables, predictive ability of post-surgical residual disease status (FIG. 11A), BMI (FIG. 11B), age (FIG. 11C), and albumin (FIG. 11D) for survival in 224 women with ovarian cancer (negative predictive values reflect a beneficial influence on survival, positive values reflect a negative influence on survival).

FIG. 12 shows a ten-year survival curve of 224 women with ovarian cancer who underwent surgery. Participants were divided into four quartiles (Q1-Q4) based on the following clinical variables: age, BMI, ASA score, albumin, FIGO, serous histology, surgical complexity, residual disease, and ascites, and frailty index (deficit accumulation). Q1 represents participants with lowest/favorable values and Q4 represents participants with highest/unfavorable healthy values.

FIGS. 13A - 13D. In statistical models limited to clinical variables plus the frailty index, predictive ability of post-surgical residual disease status (FIG. 13A), frailty index (FIG. 13B), BMI (FIG. 13C), and age (FIG. 13D) for survival in 224 women with ovarian cancer (negative predictive values reflect a beneficial influence on survival, positive values reflect a negative influence on survival).

FIG. 14 shows a ten-year survival curve of 224 women with ovarian cancer who underwent surgery. Participants were divided into four quartiles (Q1-Q4) based on age, BMI, and 25 biomarkers of aging. Q1 represents participants with lowest values and Q4 represents participants with highest values.

FIGS. 15A - 15D. In statistical models limited to age, bmi, and biomarkers, predictive ability of plasma concentrations of TNFR1 (FIG. 15A), GDF15 (FIG. 15B), FAS (FIG. 15C), and IL6 (FIG. 15D) for survival in 224 women with ovarian cancer (negative predictive values reflect a beneficial influence on survival, positive values reflect a negative influence on survival).

FIG. 16 show a ten-year survival curve, starting 90 days after surgery, of 224 women with ovarian cancer who underwent surgery. Participants were divided into four quartiles (Q1-Q4) based on age, BMI, frailty index, ASA score, albumin, FIGO, serous histology, surgical complexity, residual disease, ascites and 25 biomarkers of aging. Q1 represents participants with lowest/most favorable values and Q4 represents participants with highest values/least favorable values.

FIGS. 17A - 17D. In statistical models inclusive of age, BMI, frailty index, ASA score, albumin, FIGO, serous histology, surgical complexity, residual disease, ascites and 25 biomarkers of aging, predictive ability of plasma concentrations of TNFR1 (FIG. 17A), FAS (FIG. 17B), GDF15 (FIG. 17C), and residual disease status (FIG. 17D) for survival in 224 women with ovarian cancer (negative predictive values reflect a beneficial influence on survival, positive values reflect a negative influence on survival).

FIG. 18 shows a ten-year survival curve, starting 90 days after surgery, of 224 women with ovarian cancer who underwent surgery. Participants were divided into four quartiles (Q1-Q4) based on age, BMI, frailty index, ASA score, albumin, FIGO, serous histology, ascites and 25 biomarkers of aging (note: surgical complexity, residual disease are removed in this model). Q1 represents participants with lowest/most favorable values and Q4 represents participants with highest values/least favorable values.

FIGS. 19A - 19D. In statistical models inclusive of age, BMI, frailty index, ASA score, albumin, FIGO, serous histology, ascites and 25 biomarkers of aging (note: surgical complexity, residual disease are removed in this model), predictive ability of plasma concentrations of TNFR1 (FIG. 19A), FAS (FIG. 19B), GDF15 (FIG. 19C), and residual disease status (FIG. 19D) for survival in 224 women with ovarian cancer (negative predictive values reflect a beneficial influence on survival, positive values reflect a negative influence on survival).

FIG. 20. Using penalized COX models, ten-year survival curves of 224 women with ovarian cancer who underwent surgery. Participants were divided into four quartiles (Q1-Q4) based on age, BMI, and seven biomarkers of aging (GDF15, TNFR1, FAS, ACTIVIN A, IL-15, OPN, and MIP1A). Q1 represents participants with lowest values and Q4 represents participants with highest values.

FIG. 21. Using penalized COX models, ten-year survival curves of 224 women with ovarian cancer who underwent surgery. Participants were divided into four quartiles (Q1-Q4) based on just the seven biomarkers of aging (GDF15, TNFR1, FAS, ACTIVIN A, IL-15, OPN, and MIP1A). Q1 represents participants with lowest values and Q4 represents participants with highest values.

FIG. 22. Using penalized COX models, ten-year survival curves of 224 women with ovarian cancer who underwent surgery. Participants were divided into four quartiles (Q1-Q4) based on IL15 plus GDF15, TNFR1, FAS, ACTIVIN A, OPN, and/or MIP1A. Q1 represents participants with lowest values and Q4 represents participants with highest values.

### DETAILED DESCRIPTION

This document provides methods and materials related to assessing biological aging. In some cases, this document provides methods and materials for identifying a mammal (e.g., a human) as having an advanced biological age, as being at risk of developing one or more adverse outcomes (e.g., adverse outcomes associated with medical intervention at an advanced biological age) following a medical intervention, and/or as being likely to be responsive to one or more senotherapeutic agents. As described herein, a panel that includes four, five, six, or seven of the following SASP factors can be used to identify an advanced biological age and can predict risk for adverse outcomes (e.g., surgical complications, ICU admission, rehospitalization, and/or mortality) in older adults in response to surgical intervention(s): (1) GDF15, (2) FAS, (3) OPN, (4) TNFR1, (5) ACTIVIN A, (6) CCL3, and (7) IL15. For example, an elevated level of expression of one or more (e.g., four, five, six, or seven) SASP polypeptides can be present in a sample obtained from a mammal (e.g., a human) having an advanced biological age, at risk of developing one or more adverse outcomes following a medical intervention, and/or likely to be responsive to one or more senotherapeutic agents. In some cases, a mammal (e.g., a human) can be identified as having an advanced biological age, as being at risk of developing one or more adverse outcomes following a medical intervention, and/or as being likely to be responsive to one or more senotherapeutic agents based, at least in part, on the presence of an elevated level of expression of one or more (e.g., four, five, six, or seven) SASP polypeptides in a sample obtained from the mammal. In some cases, this document provides methods and materials for treating a mammal (e.g., a mammal identified as having an advanced biological age, as being at risk of developing one or more adverse outcomes following a medical intervention, and/or as being likely to be responsive to one or more senotherapeutic agents) undergoing one or more medical interventions (e.g., surgery) to improve outcomes for the mammal following the medical intervention(s). For example, one or more senotherapeutic agents can be administered to a mammal (e.g., a human) identified as having an advanced biological age, as being at risk of developing one or more adverse outcomes (e.g., adverse outcomes associated with medical intervention at an advanced biological age) following a medical intervention, and/or as being likely to be responsive to one or more senotherapeutic agents as described herein (e.g., based, at least in part, on the presence of an elevated level of expression of one or more (e.g., four, five, six, or seven) SASP polypeptides in a sample from the mammal) to reduce the risk of that mammal developing one or more adverse outcomes.

In some cases, the presence of an elevated level of expression of one or more (e.g., one, two, three, four, five, six, seven, or more) SASP polypeptides in a sample (e.g., a sample obtained from a mammal such as a human) can be used to identify the mammal as having an advanced biological age, as being at risk of developing one or more adverse outcomes (e.g., adverse outcomes associated with medical intervention at an advanced biological age) following a medical intervention, and/or as being likely to be responsive to one or more senotherapeutic agents. The term “elevated level” as used herein with respect to a level of expression of a SASP polypeptide refers to any level that is greater than a reference level of expression of that SASP polypeptide in a mammal (e.g., a human). The term “reference level” as used herein with respect to expression of a SASP polypeptide refers to the level of expression of the SASP polypeptide typically observed in a sample (e.g., a control sample) from one or more comparable mammals (e.g., humans of comparable chronological age) that have chronological and biological ages that match or are within 5 years of each other. In some cases, the values set forth in Table 1 can be used as reference levels for the indicated SASP polypeptide and chronological age.

TABLE 1

Mean levels (with standard deviation) of SASP polypeptide serum concentrations (pg/mL) based on chronological age
chronological age

| | | | | | | | |
|---|---|---|---|---|---|---|---|
| | 20 | 30 | 40 | 50 | 60 | 70 | 80 |
| GDF15 | 492.8 (± 192.2) | 642.3 (± 255.3) | 733.9 (± 292.9) | 865.8 (± 243.2) | 1055 (± 399.5) | 1852 (± 879.6) | 2207 (± 897.5) |
| FAS | 6421 (± 2032) | 7056 (± 2023) | 6998 (± 2696) | 7731 (± 1951) | 9257 (± 2743) | 9103 (± 2351) | 9920 (± 3363) |
| OPN | 24522 (± 11467) | 23311 (± 13751) | 24571 (± 13243) | 24467 (± 10438) | 27869 (± 9515) | 32438 (± 16490) | 35419 (± 17488) |
| TNFR1 | 3632 (± 747.5) | 3887 (± 726.1) | 4007 (± 919.8) | 4233 (± 1112) | 5322 (± 1839) | 6389 (± 2171) | 7496 (± 2797) |
| ACTIVIN A | 150.8 (± 67.22) | 180.8 (± 51.68) | 201.6 (± 62.56) | 207.3 (± 78.98) | 275.8 (± 73.85) | 336.8 (± 87.88) | 407.5 (± 106.5) |
| CCL3 | 471.7 (± 232.5) | 512.8 (± 197.0) | 634.7 (± 245.0) | 647 (± 166.8) | 697.2 (± 192.9) | 700 (± 159.7) | 744.8 (± 234.6) |
| IL15 | 0.6535 (± 0.4025) | 0.9189 (± 0.7367) | 0.8353 (± 0.5077) | 0.9532 (± 0.6931) | 0.9156 (± 0.4979) | 1.153 (± 0.4259) | 1.164 (± 0.75) |

Control samples can include, without limitation, samples from normal (e.g., healthy) mammals, samples from mammals having a chronological age of about 40 or less, samples from mammals having a chronological age of about 35 or less, samples from mammals having a chronological age of about 30 or less, and samples from mammals having a chronological age of about 25 or less. In some cases, an elevated level of expression of a SASP polypeptide can be a level that is at least 2 (e.g., at least 5, at least 10, at least 15, at least 20, at least 25, at least 35, or at least 50) fold greater than a reference level of expression of the SASP polypeptide. In some cases, when control samples have an undetectable level of expression of a SASP polypeptide, an elevated level can be any detectable level of expression of the SASP polypeptide. It will be appreciated that levels from comparable samples are used when determining whether or not a particular level is an elevated level.

The presence of an elevated level of expression of any appropriate SASP polypeptide (e.g., in a sample such as a sample obtained from a mammal such as a human) can be used to identify a mammal (e.g., a human) as having an advanced biological age, as being at risk of developing one or more adverse outcomes (e.g., adverse outcomes associated with medical intervention at an advanced biological age) following a medical intervention, and/or as being 21 likely to be responsive to one or more senotherapeutic agents. In some cases, a SASP polypeptide can be a cytokine. In some cases, a SASP polypeptide can be a chemokine. In some cases, a SASP polypeptide can be a matrix remodeling protein. In some cases, a SASP polypeptide can be a growth factor. Examples of SASP polypeptides include, without limitation, ACTIVIN A polypeptides, ADAMTS13 polypeptides, CCL3 polypeptides, CCL4 polypeptides, CCL5 polypeptides, CCL17 polypeptides, CCL22 polypeptides, FAS polypeptides, GDF15 polypeptides, GDNF polypeptides, ICAM1 polypeptides, IL6 polypeptides, IL7 polypeptides, IL8 polypeptides, IL15 polypeptides, MMP2 polypeptides, MMP9 polypeptides, OPN polypeptides, PAI1 polypeptides, PAI2 polypeptides, SOST polypeptides, TNFα polypeptides, TNFR1 polypeptides, and VEGFA polypeptides. For example, the presence of an elevated level of GDF15 polypeptides, an elevated level of FAS polypeptides, an elevated level of OPN polypeptides, an elevated level of TNFR1 polypeptides, an elevated level of ACTIVIN A polypeptides, an elevated level of CCL3 polypeptides, and/or an elevated level of IL15 polypeptides in a sample obtained from a mammal (e.g., a human) can be used to identify that mammal has having an advanced biological age, as being at risk of developing one or more adverse outcomes (e.g., adverse outcomes associated with medical intervention at an advanced biological age) following a medical intervention, and/or as being likely to be responsive to one or more senotherapeutic agents. Exemplary polypeptide sequences (and the nucleic acids encoding such polypeptides) of SASP polypeptides can be as set forth in the National Center for Biotechnology Information (NCBI) databases at, for example, Accession No. NP_004855 (Version NP_004855.2), Accession No. NP_690610 (Version NP_690610.1), and Accession No. NP_000573 (Version NP_000573.1).

When a SASP polypeptide is a GDF15 polypeptide, an elevated level of expression in a sample obtained from a human having a chronological age of about 40 years can be a level that is greater than about 750 pg/mL (e.g., greater than about 750 pg/mL, greater than about 800 pg/mL, greater than about 850 pg/mL, greater than about 900 pg/mL, greater than about 950 pg/mL, greater than about 1000 pg/mL, greater than about 1025 pg/mL, or greater than about 1050 pg/mL), an elevated level of expression in a sample obtained from a human having a chronological age of about 50 years can be a level that is greater than about 900 pg/mL (e.g., greater than about 950 pg/mL, greater than about 1000 pg/mL, greater than about 1050 pg/mL, greater than about 1100 pg/mL, or greater than about 1150 pg/mL), an elevated level of expression in a sample obtained from a human having a chronological age of about 60 years can be a level that is greater than about 1075 pg/mL (e.g., greater than about 1100 pg/mL, greater than about 1150 pg/mL, greater than about 1200 pg/mL, greater than about 1250 pg/mL, greater than about 1300 pg/mL, greater than about 1350 pg/mL, greater than about 1400 pg/mL, greater than about 1450 pg/mL, or greater than about 1500 pg/mL), and an elevated level of expression in a sample obtained from a human having a chronological age of about 70 years can be a level that is greater than about 1900 pg/mL (e.g., greater than about 1950 pg/mL, greater than about 2000 pg/mL, greater than about 2150 pg/mL, greater than about 2200 pg/mL, greater than about 2250 pg/mL, greater than about 2300 pg/mL, greater than about 2350 pg/mL, greater than about 2400 pg/mL, greater than about 2450 pg/mL, greater than about 2500 pg/mL, greater than about 2550 pg/mL, greater than about 2600 pg/mL, greater than about 2650 pg/mL, greater than about 2700 pg/mL, or greater than about 2750 pg/mL).

When a SASP polypeptide is a FAS polypeptide, an elevated level of expression in a sample obtained from a human having a chronological age of about 40 years can be a level that is greater than about 7000 pg/mL (e.g., greater than about 7500 pg/mL, greater than about 8000 pg/mL, greater than about 8500 pg/mL, greater than about 9000 pg/mL, greater than about 9500 pg/mL, or greater than about 10,000 pg/mL), an elevated level of expression in a sample obtained from a mammal having a chronological age of about 50 years can be a level that is greater than about 7750 pg/mL (e.g., greater than about 7800 pg/mL, greater than about 7900 pg/mL, greater than about 8000 pg/mL, greater than about 8500 pg/mL, greater than about 9000 pg/mL, greater than about 9500 pg/mL, or greater than about 10,000 pg/mL), an elevated level of expression in a sample obtained from a mammal having a chronological age of about 60 years can be a level that is greater than about 9500 pg/mL (e.g., greater than about 9500 pg/mL, greater than about 10,000 pg/mL, greater than about 10,500 pg/mL, greater than about 11,000 pg/mL, greater than about 11,500 pg/mL, greater than about 12,000 pg/mL, or greater than about 12,500 pg/mL), and an elevated level of expression in a sample obtained from a mammal having a chronological age of about 70 years can be a level that is greater than about 9500 pg/mL (e.g., greater than about 9500 pg/mL, greater than about 10,000 pg/mL, greater than about 10,500 pg/mL, greater than about 11,000 pg/mL, greater than about 11,500 pg/mL, greater than about 12,000 pg/mL, or greater than about 12,500 pg/mL).

When a SASP polypeptide is an OPN polypeptide, an elevated level of expression in a sample obtained from a human having a chronological age of about 40 years can be a level that is greater than about 25,000 pg/mL (e.g., greater than about 26,000 pg/mL, greater than about 27,000 pg/mL, greater than about 28,000 pg/mL, greater than about 29,000 pg/mL, greater than about 30,000 pg/mL, greater than about 31,000 pg/mL, greater than about 32,000 pg/mL, greater than about 33,000 pg/mL, greater than about 34,000 pg/mL, greater than about 35,000 pg/mL, greater than about 36,000 pg/mL, greater than about 37,000 pg/mL, or greater than about 38,000 pg/mL), an elevated level of expression in a sample obtained from a mammal having a chronological age of about 50 years can be a level that is greater than about 25,000 pg/mL (e.g., greater than about 26,000 pg/mL, greater than about 27,000 pg/mL, greater than about 28,000 pg/mL, greater than about 29,000 pg/mL, greater than about 30,000 pg/mL, greater than about 31,000 pg/mL, greater than about 32,000 pg/mL, greater than about 33,000 pg/mL, greater than about 34,000 pg/mL, greater than about 35,000 pg/mL, greater than about 36,000 pg/mL, greater than about 37,000 pg/mL, or greater than about 38,000 pg/mL), an elevated level of expression in a sample obtained from a mammal having a chronological age of about 60 years can be a level that is greater than about 28,000 pg/mL (e.g., greater than about 29,000 pg/mL, greater than about 30,000 pg/mL, greater than about 31,000 pg/mL, greater than about 32,000 pg/mL, greater than about 33,000 pg/mL, greater than about 34,000 pg/mL, greater than about 35,000 pg/mL, greater than about 36,000 pg/mL, greater than about 37,000 pg/mL, or greater than about 38,000 pg/mL), and an elevated level of expression in a sample obtained from a mammal having a chronological age of about 70 years can be a level that is greater than about 35,000 pg/mL (e.g., greater than about 36,000 pg/mL, greater than about 37,000 pg/mL, greater than about 38,000 pg/mL, greater than about 39,000 pg/mL, greater than about 40,000 pg/mL, greater than about 41,000 pg/mL, greater than about 42,000 pg/mL, greater than about 43,000 pg/mL, greater than about 44,000 pg/mL, greater than about 45,000 pg/mL, greater than about 46,000 pg/mL, greater than about 47,000 pg/mL, greater than about 48,000 pg/mL, or greater than about 49,000 pg/mL).

When a SASP polypeptide is a TNFR1 polypeptide, an elevated level of expression in a sample obtained from a human having a chronological age of about 40 years can be a level that is greater than about 4100 pg/mL (e.g., greater than about 4200 pg/mL, greater than about 4300 pg/mL, greater than about 4400 pg/mL, greater than about 4500 pg/mL, greater than about 4600 pg/mL, greater than about 4700 pg/mL, greater than about 4800 pg/mL, greater than about 4900 pg/mL, or greater than about 5000 pg/mL), an elevated level of expression in a sample obtained from a mammal having a chronological age of about 50 years can be a level that is greater than about 4500 pg/mL (e.g., greater than about 4600 pg/mL, greater than about 4700 pg/mL, greater than about 4800 pg/mL, greater than about 4900 pg/mL, greater than about 5000 pg/mL, greater than about 5100 pg/mL, greater than about 5200 pg/mL, greater than about 5300 pg/mL, or greater than about 5400 pg/mL), an elevated level of expression in a sample obtained from a mammal having a chronological age of about 60 years can be a level that is greater than about 5500 pg/mL (greater than about 6000 pg/mL, greater than about 6100 pg/mL, greater than about 6200 pg/mL, greater than about 6300 pg/mL, greater than about 6400 pg/mL, greater than about 6500 pg/mL, greater than about 6600 pg/mL, greater than about 6700 pg/mL, greater than about 6800 pg/mL, greater than about 6900 pg/mL, greater than about 7000 pg/mL, greater than about 7100 pg/mL, or greater than about 7200 pg/mL), and an elevated level of expression in a sample obtained from a mammal having a chronological age of about 70 years can be a level that is greater than about 6500 pg/mL (e.g., greater than about 7000 pg/mL, greater than about 7500 pg/mL, greater than about 8000 pg/mL, greater than about 8500 pg/mL, or greater than about 9000 pg/mL).

When a SASP polypeptide is an ACTIVIN A polypeptide, an elevated level of expression in a sample obtained from a human having a chronological age of about 40 years can be a level that is greater than about 210 pg/mL (e.g., greater than about 220 pg/mL, greater than about 230 pg/mL, greater than about 240 pg/mL, greater than about 250 pg/mL, greater than about 260 pg/mL, greater than about 270 pg/mL, or greater than about 280 pg/mL), an elevated level of expression in a sample obtained from a mammal having a chronological age of about 50 years can be a level that is greater than about 210 pg/mL (e.g., greater than about 220 pg/mL, greater than about 230 pg/mL, greater than about 240 pg/mL, greater than about 250 pg/mL, greater than about 260 pg/mL, greater than about 270 pg/mL, greater than about 280 pg/mL, greater than about 290 pg/mL, or greater than about 300 pg/mL), an elevated level of expression in a sample obtained from a mammal having a chronological age of about 60 years can be a level that is greater than about 280 pg/mL (e.g., greater than about 290 pg/mL, greater than about 300 pg/mL, greater than about 310 pg/mL, greater than about 320 pg/mL, greater than about 330 pg/mL, greater than about 340 pg/mL, greater than about 350 pg/mL, greater than about 360 pg/mL, or greater than about 370 pg/mL), and an elevated level of expression in a sample obtained from a mammal having a chronological age of about 70 years can be a level that is greater than about 350 pg/mL (e.g., greater than about 360 pg/mL, greater than about 370 pg/mL, greater than about 380 pg/mL, greater than about 390 pg/mL, greater than about 400 pg/mL, greater than about 410 pg/mL, greater than about 420 pg/mL, greater than about 430 pg/mL, or greater than about 440 pg/mL).

When a SASP polypeptide is a CCL3 polypeptide, an elevated level of expression in a sample obtained from a human having a chronological age of about 40 years can be a level that is greater than about 650 pg/mL (e.g., greater than about 700 pg/mL, greater than about 750 pg/mL, greater than about 800 pg/mL, greater than about 850 pg/mL, greater than about 900 pg/mL, or greater than about 950 pg/mL), an elevated level of expression in a sample obtained from a mammal having a chronological age of about 50 years can be a level that is greater than about 650 pg/mL (e.g., greater than about 700 pg/mL, greater than about 750 pg/mL, greater than about 800 pg/mL, greater than about 850 pg/mL, or greater than about 900 pg/mL), an elevated level of expression in a sample obtained from a mammal having a chronological age of about 60 years can be a level that is greater than about 700 pg/mL (e.g., greater than about 750 pg/mL, greater than about 800 pg/mL, greater than about 850 pg/mL, greater than about 900 pg/mL, or greater than about 950 pg/mL), and an elevated level of expression in a sample obtained from a mammal having a chronological age of about 70 years can be a level that is greater than about 700 pg/mL (e.g., greater than about 750 pg/mL, greater than about 800 pg/mL, greater than about 850 pg/mL, greater than about 900 pg/mL, or greater than about 950 pg/mL).

When a SASP polypeptide is an IL15 polypeptide, an elevated level of expression in a sample obtained from a human having a chronological age of about 40 years can be a level that is greater than about 0.85 pg/mL (e.g., greater than about 0.90 pg/mL, greater than about 0.95 pg/mL, greater than about 1.00 pg/mL, greater than about 1.05 pg/mL, greater than about 1.10 pg/mL, greater than about 1.15 pg/mL, greater than about 1.20 pg/mL, greater than about 1.25 pg/mL, greater than about 1.30 pg/mL, greater than about 1.35 pg/mL, or greater than about 1.40 pg/mL), an elevated level of expression in a sample obtained from a mammal having a chronological age of about 50 years can be a level that is greater than about 0.96 pg/mL (e.g., greater than about 1.00 pg/mL, greater than about 1.10 pg/mL, greater than about 1.20 pg/mL, greater than about 1.30 pg/mL, greater than about 1.40 pg/mL, greater than about 1.50 pg/mL, greater than about 1.60 pg/mL, greater than about 1.70 pg/mL, or greater than about 1.80 pg/mL), an elevated level of expression in a sample obtained from a mammal having a chronological age of about 60 years can be a level that is greater than about 0.95 pg/mL (e.g., greater than about 1.00 pg/mL, greater than about 1.10 pg/mL, greater than about 1.20 pg/mL, greater than about 1.30 pg/mL, greater than about 1.40 pg/mL, greater than about 1.50 pg/mL, greater than about 1.60 pg/mL, or greater than about 1.70 pg/mL), and an elevated level of expression in a sample obtained from a mammal having a chronological age of about 70 years can be a level that is greater than about 0.95 pg/mL (e.g., greater than about 1.00 pg/mL, greater than about 1.10 pg/mL, greater than about 1.20 pg/mL, greater than about 1.30 pg/mL, greater than about 1.40 pg/mL, greater than about 1.50 pg/mL, greater than about 1.60 pg/mL, or greater than about 1.70 pg/mL).

Any appropriate mammal can be assessed and/or treated as described herein. In some cases, a mammal (e.g., a human) can have experienced one or more age-related diseases, age-related dysfunctions, and/or age-related conditions (e.g., cardiovascular disease and cancer). In some cases, a mammal (e.g., a human) can have a young chronological age (e.g., can be less than about 50 years of age, less than about 40 years of age, less than about 45 years of age, less than about 40 years of age, less than about 35 years of age, less than about 30 years of age, or less than about 25 years of age). In some cases, a mammal that can be assessed and/or treated as described herein can be a human that has survived a childhood cancer. In some cases, a mammal that can be assessed and/or treated as described herein can be a human that is overweight (e.g., a human that is obese). Examples of mammals that can be assessed and/or treated as described herein include, without limitation, humans, non-human primates such as monkeys, dogs, cats, horses, cows, pigs, sheep, mice, and rats.

Any appropriate sample from a mammal (e.g., a human) can be assessed as described herein (e.g., for the presence, absence, or level of expression of one or more (e.g., four, five, six, or seven) SASP polypeptides). In some cases, a sample can be a biological sample. In some cases, a sample can contain one or more biological molecules (e.g., nucleic acids such as DNA and RNA, polypeptides, carbohydrates, lipids, hormones, and/or metabolites). Examples of samples that can be assessed as described herein include, without limitation, fluid samples (e.g., whole blood, serum, plasma, urine, and cerebrospinal fluid) and tissue samples (e.g., skeletal muscle tissue, adipose tissue, liver tissue, kidney tissue, and bone tissue) such as biopsy samples. In some cases, a sample can be a fluid sample (e.g., a blood sample such as serum or plasma). In some cases, a sample is not a tissue sample. A biological sample can be a fresh sample or a fixed sample (e.g., a formaldehyde-fixed sample or a formalin-fixed sample). In some cases, a biological sample can be a processed sample (e.g., to isolate or extract one or more biological molecules). For example, a blood (e.g., plasma) sample can be obtained from a mammal (e.g., a human) and can be assessed for the presence, absence, or level of expression of one or more (e.g., four, five, six, or seven) SASP polypeptides to determine if the mammal has an advanced biological age, is at risk of developing one or more adverse outcomes (e.g., adverse outcomes associated with medical intervention at an advanced biological age) following a medical intervention, and/or is likely to be responsive to one or more senotherapeutic agents based, at least in part, on the presence of an elevated level of expression of one or more (e.g., four, five, six, or seven) SASP polypeptides in the sample.

Any appropriate method can be used to detect the presence, absence, or level of expression of one or more (e.g., four, five, six, or seven) SASP polypeptides within a sample (e.g., a sample obtained from a mammal such as a human). In some cases, the presence, absence, or level of expression of one or more (e.g., four, five, six, or seven) SASP polypeptides within a sample can be determined by detecting the presence, absence, or level of one or more (e.g., four, five, six, or seven) SASP polypeptides in the sample. For example, immunoassays (e.g., enzyme-linked immunosorbent assays (ELISAs), multiplex assays using combinations of analyte-specific antibodies and/or aptamers, oligo-linked antibody pairs,and western blotting techniques) and mass spectrometry techniques (e.g., proteomics-based mass spectrometry assays or targeted quantification-based mass spectrometry assays) can be used to determine the presence, absence, or level of one or more (e.g., four, five, six, or seven) SASP polypeptides in a sample. When an immunoassay is used to determine the presence, absence, or level of one or more (e.g., four, five, six, or seven) SASP polypeptides in a sample, the immunoassay can use any appropriate antibody. Examples of antibodies that can be used in an immunoassay to determine the presence, absence, or level of one or more (e.g., four, five, six, or seven) SASP polypeptides in a sample include, without limitation, anti-GDF15 antibody [6D12.H10.E4] (ab189358; Abcam), anti-TNF Receptor I antibody (ab19139; Abcam), anti-osteopontin antibody (ab69498; Abcam), and anti-Activin A antibody [MM0074-7L18] (ab89307; Abcam). In some cases, an antibody that can be used in an immunoassay to determine the presence, absence, or level of one or more (e.g., four, five, six, or seven) SASP polypeptides in a sample can be as described elsewhere (see, e.g., Li et al., Mol. Cell. Biol., 38:N/A(2018); Hu et al., Genes Dev., 32:1344-1357 (2018); Wang et al., EMBO Mol. Med., 9:1150-1164 (2017); Tian et al., J. Ethnopharmacol., 232:227-235 (2019); Xu et al., Food Funct., 10:1302-1316 (2019); Yang et al., Biochem. Biophys. Res. Commun., 511:780-786 (2019); Gu et al., Am. J. Transl. Res., 11 :2603-2615 (2019); Li et al., Cell Death Dis., 10:805 (2019); Li et al., Aging, 11:6983-6998 (2019); Tsigkou et al., Reprod. Sci., 22:1597-602 (2015); Karve et al., PLoS One, 7:e37697 (2012); and Lonardo et al., Cell Cycle, 11:1282-90 (2012)). In some cases, the presence, absence, or level of expression of one or more (e.g., four, five, six, or seven) SASP polypeptides within a sample can be determined by detecting the presence, absence, or level of mRNA encoding a SASP polypeptide in the sample. For example, polymerase chain reaction (PCR)-based techniques such as quantitative reverse transcription (RT)-PCR (qPCR) techniques and RNA sequencing can be used to determine the presence, absence, or level of mRNA encoding a SASP polypeptide in the sample. In some cases, the presence, absence, or level of expression of one or more (e.g., four, five, six, or seven) SASP polypeptides within a sample can be determined by qPCR. In some cases, the presence, absence, or level of expression of one or more (e.g., four, five, six, or seven) SASP polypeptides within a sample can be determined as described in Example 1.

When the presence, absence, or level of expression of two or more (e.g., two, three, four, five, six, seven, or more) SASP polypeptides within a sample (e.g., a sample obtained from a mammal such as a human) is being detected, the presence, absence, or level of each SASP polypeptide can be detected in separate assays or in a single assay (e.g., a multiplexed assay). For example, the presence, absence, or level of expression of each SASP polypeptide within a sample can be detected in a single assay using a multiplexed bead-based assay (e.g., a multiplexed bead-based immunoassay). For example, the presence, absence, or level of mRNA encoding each SASP polypeptide within a sample can be detected in a single using a multiplexed qPCR assay (e.g., a qPCR assay performed using a multi-well plate).

In some cases, the presence of an elevated level of expression of one or more (e.g., four, five, six, or seven) SASP polypeptides in a sample obtained from a mammal (e.g., a human) can be used to identify that mammal as having an advanced biological age. As used herein, an advanced biological age is a biological age which is greater than the chronological age of a mammal (e.g., a human). In some cases, a mammal (e.g., a human) having an advanced biological age can have a frailty index of greater than about 0.15. In some cases, a frailty index can be determined as described elsewhere (see, e.g., Narasimhulu et al, Gynecol Oncol., S0090-8258(20)31131-8 (2020); Evans et al., Age and Ageing, 43(1):127-32 (2014); and Drubbel et al., J. Gerontology, 68(3):301-8 (2013)). For example, the presence of an elevated level of GDF15 polypeptides, the presence of an elevated level of FAS polypeptides, the presence of an elevated level of OPN polypeptides, the presence of an elevated level of TNFR1 polypeptides, the presence of an elevated level of ACTIVIN A polypeptides, the presence of an elevated level of CCL3 polypeptides, and/or the presence of an elevated level of IL15 polypeptides in a sample obtained from a mammal (e.g., a human) can be used to identify that mammal as having an advanced biological age.

In some cases, the absence of an elevated level of expression of one or more (e.g., four, five, six, or seven) SASP polypeptides (e.g., the presence of a reference level of one or more SASP polypeptides) in a sample obtained from a mammal (e.g., a human) can be used to identify that mammal as not having an advanced biological age (e.g., as lacking an advanced biological age). For example, the absence of an elevated level of GDF15 polypeptides, the absence of an elevated level of FAS polypeptides, the absence of an elevated level of OPN polypeptides, the absence of an elevated level of TNFR1 polypeptides, the absence of an elevated level of ACTIVIN A polypeptides, the absence of an elevated level of CCL3 polypeptides, and/or the absence of an elevated level of IL15 polypeptides in a sample obtained from a mammal (e.g., a human) can be used to identify that mammal as not having an advanced biological age or as having chronological and biological ages that match or are within about 5 years of each other.

In some cases, the presence of an elevated level of expression of one or more (e.g., four, five, six, or seven) SASP polypeptides in a sample obtained from a mammal (e.g., a human) can be used to identify that mammal as being at risk of developing one or more adverse outcomes (e.g., adverse outcomes associated with medical intervention at an advanced biological age) following a medical intervention. An adverse outcome following a medical intervention can be any type of adverse outcome following a medical intervention. In some cases, an adverse outcome following a medical intervention can be associated with an advanced biological age. Examples of adverse outcomes following a medical intervention such as surgery include, without limitation, ICU admission (e.g., ICU admission to the ICU within 30 days of surgery), rehospitalization (e.g., rehospitalization within 12 months of hospital discharge), experiencing an adverse event (e.g., experiencing an adverse event within 12 months of surgery), and mortality. Examples of adverse events that can occur following a medical intervention such as surgery (e.g., adverse post-operative events) include, without limitation, myocardial infarction, new arrhythmia, new conduction abnormality, stroke, deep venous thrombosis, pulmonary emboli, pneumonia, plural effusion, new renal insufficiency, GI bleeding, new seizure disorder, significant hypotension, significant tachycardia, significant bradycardia, urinary tract infection, other infection, acute dementia, vascular complication, and acute kidney injury,. A medical intervention can be any type of medical intervention. In some cases, a medical intervention can be surgery. Examples of medical interventions that can be followed by an adverse outcome include, without limitation, cytoreductive surgery, cardiovascular surgeries, (e.g., surgery for severe aortic stenosis), orthopedic surgeries (e.g., hip replacement), organ transplant (e.g., lung, liver, kidney, heart, and combinations thereof), and gynecologic surgeries. Examples of adverse outcomes following a medical intervention such as drug interventions include, without limitation, drug-related toxicity and an inability to complete a full regimen of therapy (e.g., recommended cycles of chemotherapy). For example, the presence of an elevated level of GDF 15 polypeptides, the presence of an elevated level of FAS polypeptides, the presence of an elevated level of OPN polypeptides, the presence of an elevated level of TNFR1 polypeptides, the presence of an elevated level of ACTIVIN A polypeptides, the presence of an elevated level of CCL3 polypeptides, and/or the presence of an elevated level of IL15 polypeptides in a sample obtained from a mammal (e.g., a human) can be used to identify that mammal as being at risk of developing one or more adverse outcomes (e.g., adverse outcomes associated with medical intervention at an advanced biological age) following a medical intervention.

In some cases, the absence of an elevated level of expression of one or more (e.g., four, five, six, or seven) SASP polypeptides (e.g., the presence of a reference level of one or more SASP polypeptides) in a sample obtained from a mammal (e.g., a human) can be used to identify that mammal as being at lower risk (e.g., as not being at risk) of developing one or more adverse outcomes (e.g., adverse outcomes associated with medical intervention at an advanced biological age) following a medical intervention (e.g., as lacking a risk or as being at low risk of developing one or more adverse outcomes following a medical intervention). For example, the absence of an elevated level of GDF15 polypeptides, the absence of an elevated level of FAS polypeptides, the absence of an elevated level of OPN polypeptides, the absence of an elevated level of TNFR1 polypeptides, the absence of an elevated level of ACTIVIN A polypeptides, the absence of an elevated level of CCL3 polypeptides, and/or the absence of an elevated level of IL15 polypeptides in a sample obtained from a mammal (e.g., a human) can be used to identify that mammal as not being at risk of developing one or more adverse outcomes (e.g., adverse outcomes associated with medical intervention at an advanced biological age) following a medical intervention.

In some cases, the presence of an elevated level of expression of one or more (e.g., four, five, six, or seven) SASP polypeptides in a sample obtained from a mammal (e.g., a human) can be used to identify that mammal as being likely to be responsive to one or more senotherapeutic agents. A senotherapeutic agent can be any type of molecule (e.g., small molecules or polypeptides). In some cases, a senotherapeutic agent can be a senolytic agent (i.e., an agent having the ability to induce cell death in senescent cells). In some cases, a senotherapeutic agent can be a senomorphic agent (i.e., an agent having the ability to suppress senescent phenotypes without cell killing). Examples of senotherapeutic agents include, without limitation, dasatinib, quercetin, navitoclax, A1331852, A1155463, fisetin, luteolin, geldanamycin, tanespimycin, alvespimycin, piperlongumine, panobinostat, FOX04-related peptides, nutlin3a, ruxolitinib, metformin, and rapamycin. For example, the presence of an elevated level of GDF15 polypeptides, the presence of an elevated level of FAS polypeptides, the presence of an elevated level of OPN polypeptides, the presence of an elevated level of TNFR1 polypeptides, the presence of an elevated level of ACTIVIN A polypeptides, the presence of an elevated level of CCL3 polypeptides, and/or the presence of an elevated level of IL15 polypeptides in a sample obtained from a mammal (e.g., a human) can be used to identify that mammal as being likely to be responsive to one or more senotherapeutic agents.

In some cases, the absence of an elevated level of expression of one or more (e.g., four, five, six, or seven) SASP polypeptides (e.g., the presence of a reference level of one or more SASP polypeptides) in a sample obtained from a mammal (e.g., a human) can be used to identify that mammal as not being likely to be responsive to one or more senotherapeutic agents (e.g., as lacking responsive to one or more senotherapeutic agents). For example, the absence of an elevated level of GDF15 polypeptides, the absence of an elevated level of FAS polypeptides, the absence of an elevated level of OPN polypeptides, the absence of an elevated level of TNFR1 polypeptides, the absence of an elevated level of ACTIVIN A polypeptides, the absence of an elevated level of CCL3 polypeptides, and/or the absence of an elevated level of IL15 polypeptides in a sample obtained from a mammal (e.g., a human) can be used to identify that mammal as not being likely to be responsive to one or more senotherapeutic agents.

In some cases, the presence of an elevated level of expression of one or more (e.g., four, five, six, or seven) SASP polypeptides in a sample obtained from a mammal (e.g., a human) can be used to identify the presence of senescent cells (e.g., the presence of an enriched systemic senescent cell burden) within the mammal. As used herein, the term systemic senescent cell burden refers to the abundance of senescent cells within the organs/tissues of a mammal (e.g., a human). An enriched systemic senescent cell burden can be an abundance of senescent cells that is greater than the amount of senescent cells that typically accumulate within most organs of a healthy mammal having a comparable chronological age. A senescent cell can be any type of cell. In some cases, a senescent cell can be a post-mitotic cell. Examples of types of cells that can be senescent and whose presence within a mammal (e.g., a human) can be identified as described herein include, without limitation, endothelial cells, epithelial cells, preadipocytes, fibroblasts, myoblasts, mesenchymal stem cells, osteocytes, microglia, immune cells, cardiomyocytes, myofibers, and neurons. For example, the presence of an elevated level of GDF15 polypeptides, an elevated level of FAS polypeptides, an elevated level of OPN polypeptides, an elevated level of TNFR1 polypeptides, an elevated level of ACTIVIN A polypeptides, an elevated level of CCL3 polypeptides, and/or an elevated level of IL15 polypeptides in a sample obtained from a mammal (e.g., a human) can be used to identify the presence of an enriched systemic senescent cell burden within the mammal.

In some cases, the absence of an elevated level of expression of one or more (e.g., four, five, six, or seven) SASP polypeptides (e.g., the presence of a reference level of one or more SASP polypeptides) in a sample obtained from a mammal (e.g., a human) can be used to identify that mammal as not having a systemic senescent cell burden (e.g., as lacking a systemic senescent cell burden). For example, the absence of an elevated level of GDF15 polypeptides, the absence of an elevated level of FAS polypeptides, the absence of an elevated level of OPN polypeptides, the absence of an elevated level of TNFR1 polypeptides, the absence of an elevated level of ACTIVIN A polypeptides, the absence of an elevated level of CCL3 polypeptides, and/or the absence of an elevated level of IL15 polypeptides in a sample obtained from a mammal (e.g., a human) can be used to identify that mammal as not having an enriched systemic senescent cell burden.

This document provides methods and materials for treating a mammal (e.g., a mammal identified as having an advanced biological age, as being at risk of developing one or more adverse outcomes (e.g., adverse outcomes associated with medical intervention at an advanced biological age) following a medical intervention, and/or as being likely to be responsive to one or more senotherapeutic agents as described herein). In some cases, a mammal (e.g., a human) identified as having an advanced biological age, as being at risk of developing one or more adverse outcomes following a medical intervention, and/or as being likely to be responsive to one or more senotherapeutic agents as described herein (e.g., based, at least in part, on the presence of an elevated level of expression of one or more (e.g., four, five, six, or seven) SASP polypeptides in a sample from the mammal) and undergoing one or more medical interventions (e.g., surgery) can be administered one or more senotherapeutic agents to treat the mammal. For example, a mammal (e.g., a human) identified as having an advanced biological age, as being at risk of developing one or more adverse outcomes following a medical intervention, and/or as being likely to be responsive to one or more senotherapeutic agents as described herein can be administered or instructed to self-administer one or more (e.g., one, two, three, four, five or more) senotherapeutic agents. Any appropriate senotherapeutic agent can be used as described herein. A senotherapeutic agent that can be used as described herein can be any type of molecule (e.g., small molecules or polypeptides). In some cases, a senotherapeutic agent can be a senolytic agent (i.e., an agent having the ability to induce cell death in senescent cells). In some cases, a senotherapeutic agent can be a senomorphic agent (i.e., an agent having the ability to suppress senescent phenotypes without cell killing). Examples of senotherapeutic agents that can be used as described herein (e.g., to reduce the risk of developing adverse outcomes following a medical intervention) can include, without limitation, dasatinib, quercetin, navitoclax, A1331852, A1155463, fisetin, luteolin, geldanamycin, tanespimycin, alvespimycin, piperlongumine, panobinostat, FOX04-related peptides, nutlin3a, ruxolitinib, metformin, and rapamycin.

In some cases, a mammal (e.g., a human) identified as having an advanced biological age, as being at risk of developing one or more adverse outcomes following a medical intervention, and/or as being likely to be responsive to one or more senotherapeutic agents as described herein (e.g., based, at least in part, on the presence of an elevated level of expression of one or more (e.g., four, five, six, or seven) SASP polypeptides in a sample from the mammal) can undergo one or more lifestyle interventions to treat the mammal. For example, a mammal (e.g., a human) identified as having an advanced biological age as described herein can undergo one or more lifestyle interventions to boost resilience of the mammal prior to a medical intervention. Examples of lifestyle interventions that can be used as described herein (e.g., to reduce the risk of developing adverse outcomes following a medical intervention) can include, without limitation, change in diet and increased exercise.

In some cases, a mammal (e.g., a human) identified as having an advanced biological age as described herein (e.g., based, at least in part, on the presence of an elevated level of expression of one or more (e.g., four, five, six, or seven) SASP polypeptides in a sample from the mammal) can be treated with one or more agents used to treat frailty. For example, a mammal (e.g., a human) identified as having an advanced biological age as described herein can be administered or instructed to self-administer one or more senotherapeutic agents to reduce or eliminate one or more (e.g., one, two, three, four, five or more) symptoms of frailty. Examples of symptoms of frailty that can be reduced or eliminated as described herein include, without limitation, unintentional weight loss, exhaustion, muscle weakness, slowness while walking, low levels of activity, inflammation, and difficulties with activities of daily living. For example, one or more senotherapeutic agents can be administered to a mammal (e.g., a human) in need thereof (e.g., a mammal having an advanced biological age) as described herein to reduce one or more symptoms of frailty in the mammal by, for example, 10, 20, 30, 40, 50, 60, 70, 80, 90, 95, or more percent.

In some cases, a mammal (e.g., a human) identified as being at risk of developing one or more adverse outcomes following a medical intervention as described herein (e.g., based, at least in part, on the presence of an elevated level of expression of one or more (e.g., four, five, six, or seven) SASP polypeptides in a sample from the mammal) and undergoing one or more medical interventions (e.g., surgery) can be treated with one or more senotherapeutic agents and/or can undergo one or more lifestyle interventions to improve outcomes for the mammal following the medical intervention(s). For example, a mammal (e.g., a human) identified as having an advanced biological age as described herein can be administered or instructed to self-administer one or more senotherapeutic agents to alleviate (e.g., to reduce or eliminate) one or more (e.g., one, two, three, four, five or more) adverse event that can occur following a medical intervention (e.g., surgery). Examples of adverse events that can occur following a medical intervention such as surgery (e.g., adverse post-operative events) include, without limitation, myocardial infarction, new arrhythmia, new conduction abnormality, stroke, deep venous thrombosis, pulmonary emboli, pneumonia, plural effusion, new renal insufficiency, GI bleeding, new seizure disorder, significant hypotension, significant tachycardia, significant bradycardia, urinary tract infection, other infection, acute dementia, vascular complication, and acute kidney injury. Each of these adverse events that can occur following a medical intervention such as surgery can be identified and/or monitored using clinical techniques as described elsewhere. For example, one or more senotherapeutic agents can be administered to a mammal (e.g., a human) in need thereof (e.g., a mammal at risk of developing one or more adverse outcomes following a medical intervention) as described herein to reduce the severity of one or more adverse events that can occur following a medical intervention such as surgery in the mammal by, for example, 10, 20, 30, 40, 50, 60, 70, 80, 90, 95, or more percent.

In some cases, a mammal (e.g., a human) identified as having an advanced biological age, as being at risk of developing one or more adverse outcomes following a medical intervention, and/or as being likely to be responsive to one or more senotherapeutic agents as described herein (e.g., based, at least in part, on the presence of an elevated level of expression of one or more (e.g., four, five, six, or seven) SASP polypeptides in a sample from the mammal) can be treated with one or more senotherapeutic agents and/or can undergo one or more lifestyle interventions to reduce the number of senescent cells (e.g., to reduce a systemic senescent cell burden) within the mammal. For example, one or more senotherapeutic agents can be administered to a mammal (e.g., a human) in need thereof (e.g., a mammal having an advanced biological age, as being at risk of developing one or more adverse outcomes following a medical intervention, and/or as being likely to be responsive to one or more senotherapeutic agents) as described herein to reduce the number of senescent cells in the mammal by, for example, 10, 20, 30, 40, 50, 60, 70, 80, 90, 95, or more percent.

In some cases, a mammal (e.g., a human) identified as having an advanced biological age, as being at risk of developing one or more adverse outcomes following a medical intervention, and/or as being likely to be responsive to one or more senotherapeutic agents as described herein (e.g., based, at least in part, on the presence of an elevated level of expression of one or more (e.g., four, five, six, or seven) SASP polypeptides in a sample from the mammal) can be selected for more frequent (e.g., additional and/or increased) monitoring following a medical intervention.

In some cases, a mammal (e.g., a human) identified as having an advanced biological age, as being at risk of developing one or more adverse outcomes following a medical intervention, and/or as being likely to be responsive to one or more senotherapeutic agents as described herein (e.g., based, at least in part, on the presence of an elevated level of expression of one or more (e.g., four, five, six, or seven) SASP polypeptides in a sample from the mammal) can be selected for more robust transitional care following a medical intervention.

In some cases, the methods and materials described herein can be used for identifying one or more agents that can be used for treating a mammal (e.g., a mammal identified as having an advanced biological age, as being at risk of developing one or more adverse outcomes following a medical intervention, and/or as being likely to be responsive to one or more senotherapeutic agents). For example, a mammal can be administered a candidate agent, and the presence, absence, or level of expression of one or more (e.g., four, five, six, or seven) SASP polypeptides can be used to identify whether or not the candidate agent can be used for treating a mammal identified as having an advanced biological age, as being at risk of developing one or more adverse outcomes following a medical intervention, and/or as being likely to be responsive to one or more senotherapeutic agents. In some cases, the presence of an elevated level of expression of one or more (e.g., four, five, six, seven, or more) SASP polypeptides in a sample (e.g., a sample obtained from a mammal such as a human) can be used to determine that the candidate agent can be used for treating a mammal identified as having an advanced biological age, as being at risk of developing one or more adverse outcomes following a medical intervention, and/or as being likely to be responsive to one or more senotherapeutic agents. In some cases, the absence of an elevated level of expression of one or more (e.g., four, five, six, seven, or more) SASP polypeptides (e.g., the presence of a reference level of one or more SASP polypeptides) in a sample (e.g., a sample obtained from a mammal such as a human) can be used to determine that the candidate agent is not likely to be useful for treating a mammal identified as having an advanced biological age, as being at risk of developing one or more adverse outcomes following a medical intervention, and/or as being likely to be responsive to one or more senotherapeutic agents. For example, the presence, absence, or level of expression of one or more (e.g., four, five, six, or seven) SASP polypeptides can be used as an endpoint in a clinical trial (e.g., a clinical trial to determine whether a candidate agent has a desired mechanism of action and/or to determine whether a candidate agent can progress to a next phase of clinical trial).The invention will be further described in the following examples, which do not limit the scope of the invention described in the claims.

### EXAMPLES

### Example 1: The Senescence-Associated Secretome as an Indicator of Age and Medical Risk

This Example identifies circulating senescence-associated secretory phenotype (SASP) polypeptides associated with advanced age and/or medical risk.

### Results

### Senescent Cells Exhibit a Robust and Distinct SASP

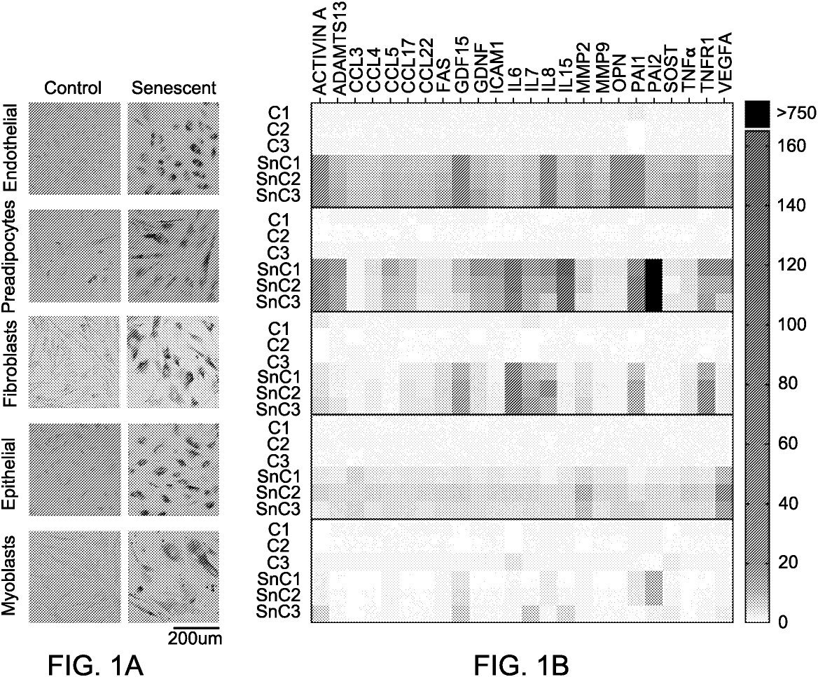

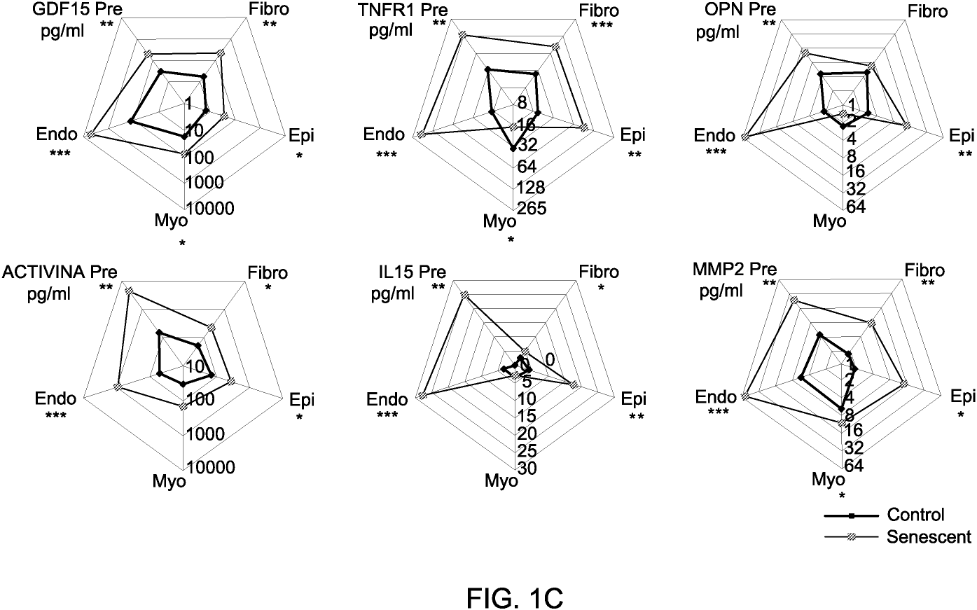

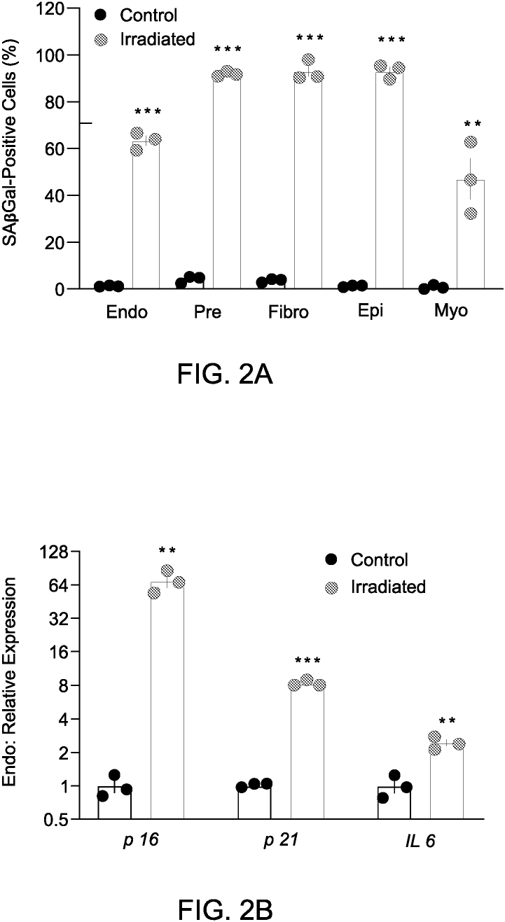

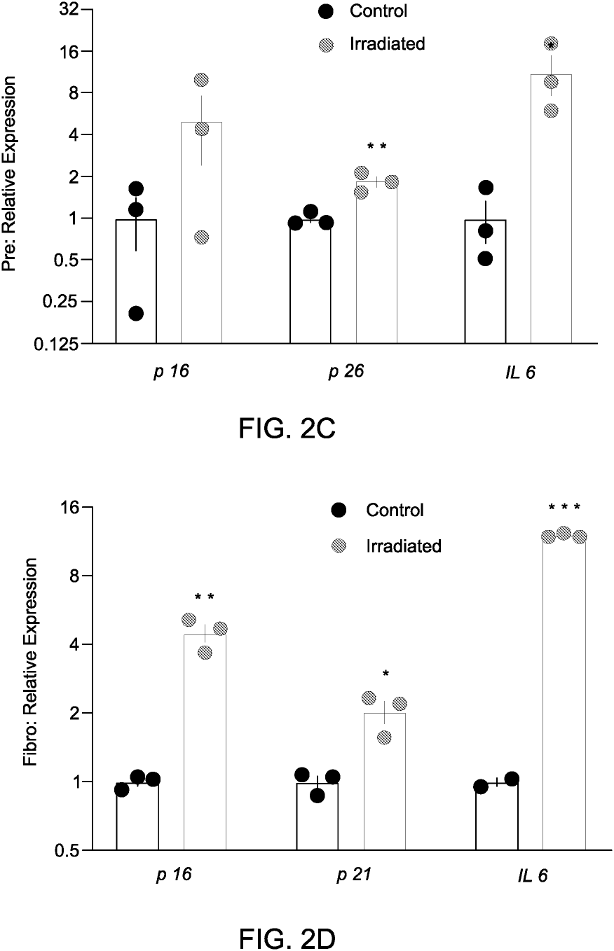

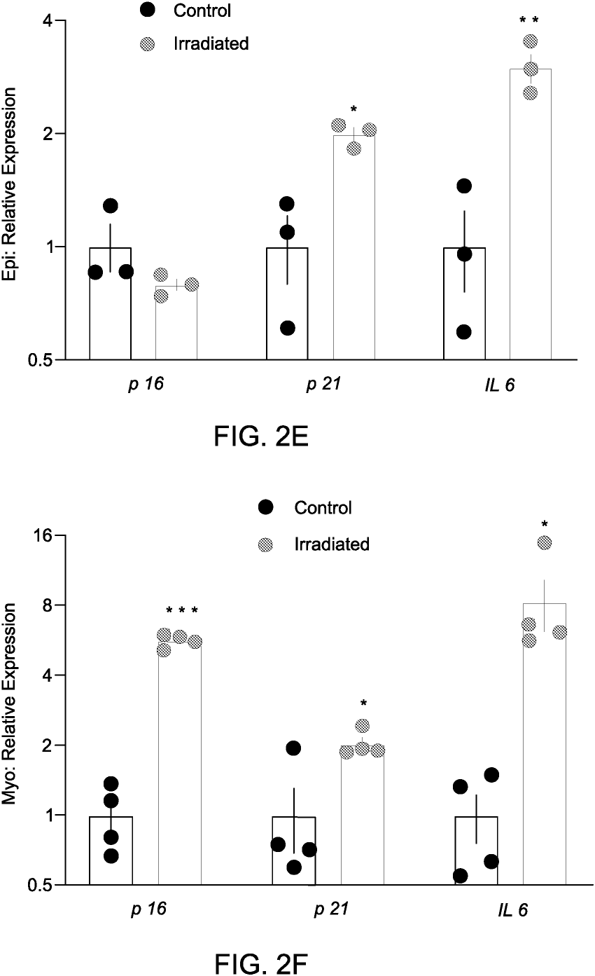

To develop a candidate panel of SASP biomarkers for human application, conditioned media were collected from five different senescent versus non-senescent human cell types: endothelial and epithelial cells, preadipocytes, fibroblasts, and myoblasts. Irradiation-induced senescence was confirmed by senescence-associated β-galactosidase (SA-β-Gal) staining and real-time PCR analysis of senescence-activated genes (FIG. 1A and FIG. 2). A biased approach, based on the molecular knowledge of the SASP obtained in model systems, was used to select candidate proteins. High levels of both distinct and overlapping SASP factors, including cytokines, chemokines, matrix remodeling proteins, and growth factors, were identified in all senescent cells assayed, relative to non-senescent cells (FIG. 1B and Table 2). Senescent endothelial cells, preadipocytes, and fibroblasts produced a more robust SASP, relative to epithelial cells and myoblasts, with distinct proteins increased per cell type (FIGS. 1B-C, Table 2 and 3). GDF15, OPN, and IL8 were abundantly produced and secreted by senescent endothelial cells, while higher levels of IL15, IL6, and ACTIVIN A were produced and secreted by senescent preadipocytes (FIGS. 1B-C and Table 3 (Appendix A)). Thus, distinct cell types throughout the body may uniquely contribute to a dynamic SASP in vivo.

TABLE 2

Fold change comparison of in vitro SASP

| | Endothelial | | Preadipocytes | | Fibroblasts | | Epithelial | | Myoblasts | |
|---|---|---|---|---|---|---|---|---|---|---|
| | Foldchange | P value | Foldchange | P value | Foldchange | P value | Foldchange | P value | Foldchange | P value |
| ACTIVIN A | 17.85 | <0.001 | 28.18 | 0.005 | 4.37 | 0.040 | 3.77 | 0.041 | 3.27 | 0.163 |
| ADAMTS13 | 9.97 | <0.001 | 12.77 | <0.001 | 2.64 | 0.063 | 3.94 | 0.008 | 0.79 | 0.896 |
| CCL3 | 7.21 | <0.001 | 1.26 | 0.173 | 1.33 | 0.361 | 5.26 | <0.001 | 0.40 | 0.020 |
| CCL4 | 7.21 | <0.001 | 3.89 | <0.001 | 2.19 | 0.001 | 4.16 | 0.007 | 0.63 | 0.229 |
| CCL5 | 8.81 | <0.001 | 9.17 | 0.001 | 1.00 | NA | 4.12 | 0.007 | 1.61 | 0.020 |
| CCL17 | 8.10 | <0.001 | 7.42 | <0.001 | 3.16 | 0.005 | 4.15 | 0.003 | 0.72 | 0.561 |
| CCL22 | 8.20 | <0.001 | 3.25 | 0.002 | 2.92 | 0.002 | 4.11 | 0.006 | 0.60 | 0.160 |
| FAS | 9.50 | <0.001 | 3.53 | <0.001 | 4.79 | <0.001 | 4.24 | 0.012 | 1.19 | 0.069 |
| GDF15 | 41.96 | <0.001 | 6.90 | 0.011 | 11.74 | <0.001 | 5.03 | 0.003 | 4.80 | 0.020 |
| GDNF | 10.33 | 0.001 | 11.67 | 0.001 | 4.06 | 0.002 | 4.16 | 0.011 | 0.75 | 0.823 |
| ICAM1 | 8.28 | <0.001 | 12.35 | <0.001 | 2.06 | 0.004 | 4.22 | 0.006 | 0.76 | 0.615 |
| IL6 | 6.67 | <0.001 | 78.96 | 0.003 | 56.51 | 0.004 | 3.84 | 0.035 | 3.23 | 0.200 |
| IL7 | 8.18 | <0.001 | 11.42 | <0.001 | 10.92 | 0.002 | 4.15 | 0.011 | 0.82 | 0.962 |
| IL8 | 46.15 | <0.001 | 14.67 | 0.065 | 12.34 | 0.029 | 3.61 | 0.048 | 3.44 | 0.056 |
| IL15 | 8.40 | <0.001 | 139.94 | <0.001 | 1.81 | 0.014 | 4.08 | 0.006 | 0.90 | 0.827 |
| MMP2 | 10.31 | <0.001 | 5.49 | 0.004 | 4.56 | 0.004 | 7.96 | 0.012 | 1.42 | 0.030 |
| MMP9 | 8.28 | <0.001 | 3.06 | 0.001 | 1.79 | 0.008 | 4.13 | 0.007 | 0.67 | 0.208 |
| OPN | 26.14 | <0.001 | 2.82 | 0.002 | 1.39 | 0.090 | 4.99 | 0.008 | 0.70 | 0.471 |
| PAI1 | 27.15 | <0.001 | 51.48 | 0.018 | 10.02 | 0.002 | 3.80 | 0.057 | 2.43 | 0.199 |
| PAI2 | 8.93 | <0.001 | 1759.00 | 0.027 | 1.00 | NA | 3.99 | <0.001 | 7.35 | 0.085 |
| SOST | 9.49 | <0.001 | 2.76 | 0.087 | 1.00 | NA | 3.82 | 0.027 | 1.58 | 0.054 |
| TNFR1 | 11.35 | <0.001 | 4.03 | 0.002 | 3.02 | <0.001 | 5.10 | 0.006 | 0.54 | 0.034 |
| TNFα | 8.18 | <0.001 | 13.41 | 0.002 | 19.70 | 0.016 | 4.32 | 0.014 | 0.84 | 0.919 |
| VEGFA | 9.07 | <0.001 | 11.22 | 0.019 | 2.37 | 0.001 | 12.24 | 0.003 | 1.76 | 0.030 |

### Circulating SASP Factors Are Associated With Advanced Chronological Age

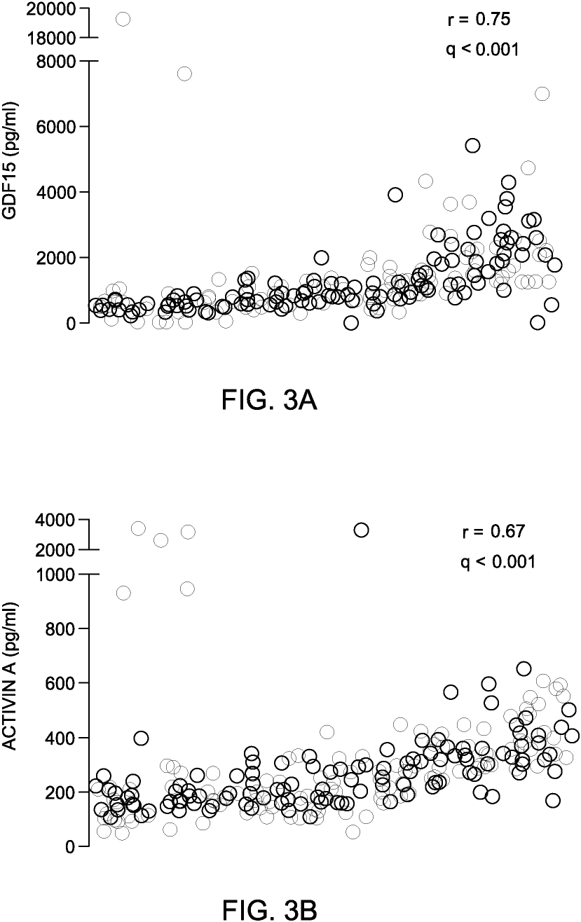

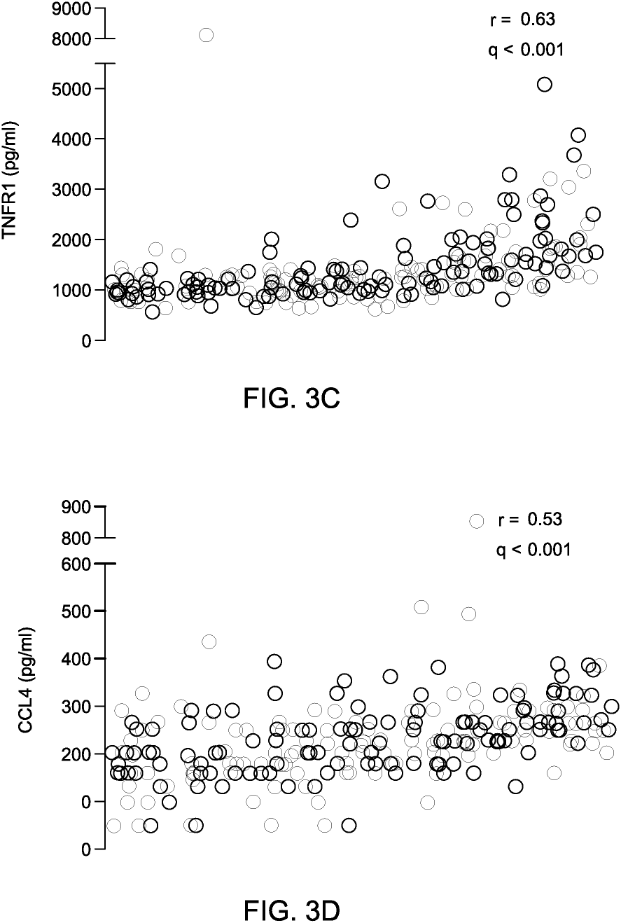

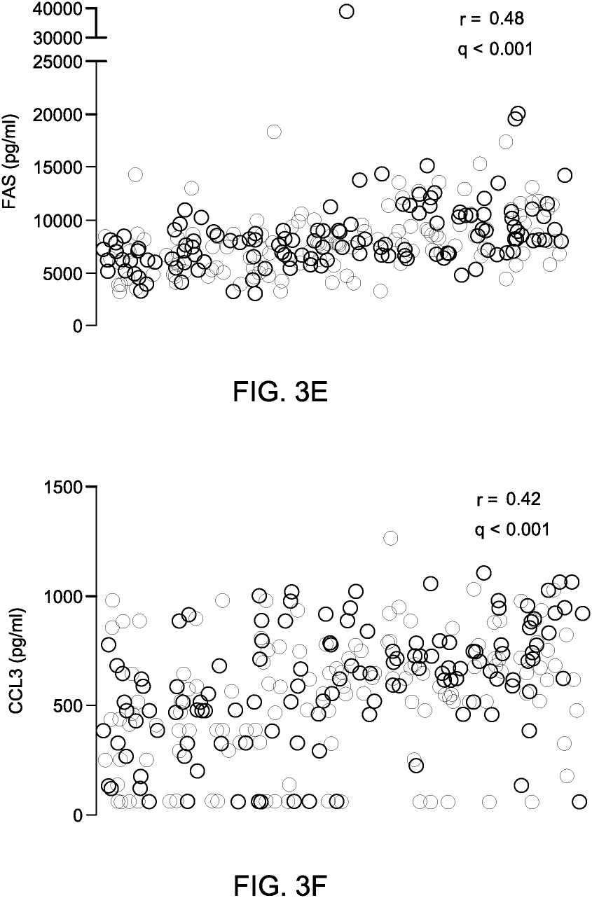

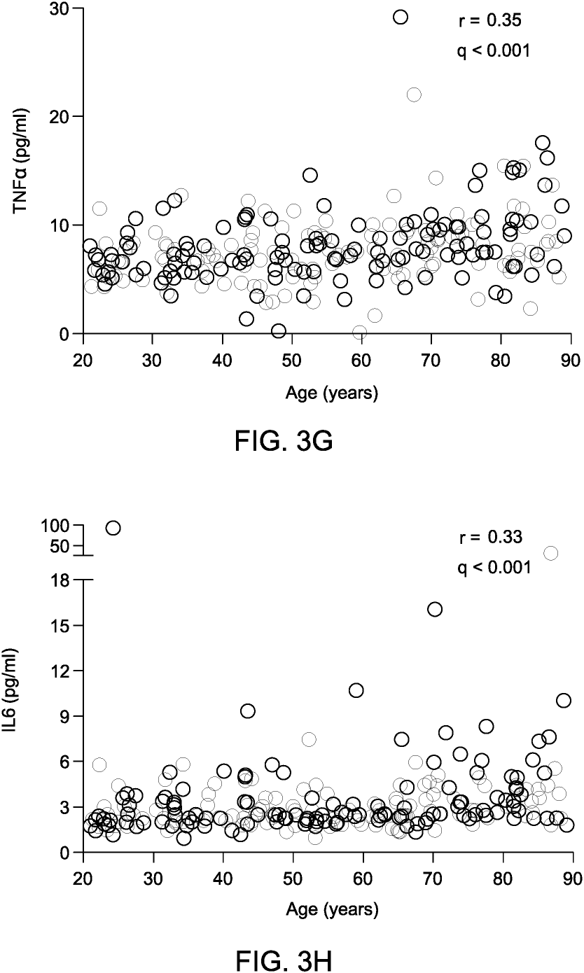

Building on the premise that senescent cells accumulate with chronological age, the panel of 24 SASP proteins identified as biologically relevant in vitro was measured in the plasma of a random sample of 267 participants. The sample was equivalently distributed by sex and age from 20-90 years (Table 4). Circulating concentrations of 19 SASP proteins were associated with chronological age, and associations between 17 SASP factors and chronological age remained significant after adjusting for sex and BMI (Table 5), highlighting the potential influence of sex and body composition on the biology of aging. In unadjusted analyses, Spearman correlation analyses indicated that GDF15 and ACTIVIN A were the strongest candidate biomarkers of chronological age, followed by TNFR1, CCL4, FAS, CCL3, TNFα, and IL6, all of which individually explained at least 10% of the variance in chronological age in unadjusted analyses (FIG. 3). GDF15, ACTIVIN A, CCL4, FAS, CCL3, and TNFα remained significantly associated with age after adjusting for sex and BMI (Table 5).

TABLE 4

Characteristics of participants used to study associations between circulating SASP and chronological age

| | 20-29 | 30-39 | 40-49 | 50-59 | 60-69 | 70-79 | 80-89 | |
|---|---|---|---|---|---|---|---|---|
| Characteristic | Number (%) or Median (Q1,Q3) | | | | | | | p-value |
| n | 38 | 37 | 38 | 39 | 37 | 38 | 40 | |
| Female | 20 (52.6%) | 19 (51.4%) | 20 (52.6%) | 20 (51.3%) | 20 (54.1%) | 18 (47.4%) | 20 (50%) | 0.810¹ |
| Male | 18 (47.4%) | 18 (48.6%) | 18 (47.4%) | 19 (48.7%) | 17 (45.9%) | 20 (52.6%) | 20 (50%) | |
| Age in years | 24.2 (23,26.1) | 33.2 (32.4,36.4) | 44.5 (43.2,47.6) | 54.6 (52.8,56.9) | 65.5 (63,67.5) | 74.4 (71.9,76.9) | 83.2 (81.7,86.3) | N/A |
| BMI | 24 (21.7,27.4) | 26.7 (24.3,28.8) | 25.8 (23.6,27.8) | 27.2 (23.4,30.3) | 28.1 (24.8,31.2) | 28.6 (24.8,31) | 27.1 (24.7,29.5) | 0.004² |
| Frailty score | 0 (0,0.03) n-miss = 9 | 0 (0,0.03) n-miss = 10 | 0.03 (0.03,0.08) n-miss = 8 | 0.06 (0,0.1) n-miss = 4 | 0.07 (0.03,0.13) n-miss = 4 | 0.1 (0.06,0.17) n-miss = 6 | 0.19 (0.1,0.27) n-miss = 10 | <0.001² |

¹Kruskal-Wallis, ²Spearman Correlation

TABLE 5

Circulating SASP factors are associated with chronological age

| | | Model 1 | | Model 2 | |
|---|---|---|---|---|---|
| Protein | Alias | r-value | q-value | r-value | q-value |
| ACTIVIN A | INHBA | 0.671 | <0.001 | 0.105 | 0.022 |
| ADAMTS13 | VWFCP | -0.163 | 0.011 | -0.184 | <0.001 |
| CCL3 | MIP1A, SCYA3 | 0.415 | 0.001 | 0.393 | <0.001 |
| CCL4 | MIP1B, SCYA4 | 0.526 | <0.001 | 0.446 | <0.001 |
| CCL5 | RANTES, SCYA5 | -0.138 | 0.031 | -0.153 | 0.001 |
| CCL17 | TARC, SCYA17 | 0.237 | <0.001 | 0.235 | <0.001 |
| CCL22 | MDC, SCYA22 | 0.187 | 0.003 | 0.155 | 0.001 |
| FAS | APT1, TNFRSF6 | 0.482 | <0.001 | 0.376 | <0.001 |
| GDF15 | MIC1, NAG1, NRG1 | 0.746 | <0.001 | 0.320 | <0.001 |
| GDNF | ATF | -0.054 | 0.425 | -0.040 | 0.416 |
| ICAM1 | CD54 | 0.192 | 0.003 | 0.082 | 0.077 |
| IL6 | IFNB2 | 0.330 | <0.001 | 0.015 | 0.759 |
| IL7 | | -0.130 | 0.041 | -0.160 | <0.001 |
| IL8 | CXCL8 | 0.198 | 0.002 | 0.106 | 0.021 |
| IL15 | | 0.267 | <0.001 | 0.127 | 0.005 |
| MMP2 | CLG4A | 0.119 | 0.061 | 0.098 | 0.031 |
| MMP9 | CLG4B | 0.037 | 0.574 | 0.013 | 0.759 |
| OPN | SPP1, PSEC0156 | 0.260 | <0.001 | 0.228 | <0.001 |
| PAI1 | SERPINE1, PLANH1 | -0.033 | 0.587 | -0.027 | 0.575 |
| PAI2 | SERPINEB2, PLANH2 | -0.042 | 0.534 | -0.032 | 0.511 |
| SOST | DAND6 | 0.303 | <0.001 | 0.280 | <0.001 |
| TNFα | TNFSF2 | 0.349 | <0.001 | 0.317 | <0.001 |
| TNFR1 | CD 120a | 0.632 | <0.001 | 0.440 | <0.001 |
| VEGFA | VPH | 0.172 | 0.007 | 0.185 | <0.001 |

Model 1: FDR-corrected spearman correlation of chronological age versus SASP protein.
Model 2: FDR-corrected spearman correlation of chronological age versus SASP protein adjusted for sex and BMI.

### Circulating SASP Factors Are Associated with Advanced Biological Age

The principal exploratory sample used to test associations between plasma levels of the panel of 24 SASP factors and biological age, as measured by the frailty index, was comprised of older adults undergoing surgery for severe aortic stenosis (n = 97). To determine whether associations between biological age and circulating SASP factors were disease-agnostic, plasma SASP factor concentrations were also assessed in a limited case-control study of older women undergoing surgery for ovarian cancer, in which women with a greater burden of age-associated deficits based on the frailty index were compared to counterparts with lower deficit burden, yet of similar age and disease severity (n = 36). Plasma SASP factor concentrations and frailty index associations were also studied in the subset of 267 Mayo Clinic biobank sample participants age 60-90 years (n = 115). Demographic information for all three samples is presented in Tables 6 and 7.

TABLE 6

Characteristics of participants used to study associations between circulating SASP and biological age

| | Aortic Stenosis | Ovarian Cancer | Biobank, 60+ | |
|---|---|---|---|---|
| Characteristic | Number (%) or Median (Q1,Q3) | | | p-value |
| n | 97 | 36 | 115 | |
| Female | 42 (43%) | 36 (100%) | 58 (50%) | <0.001¹ |
| Male | 55 (57%) | 0 (0%) | 57 (50%) | |
| Age in years | 82.0 (76.0, 87.0) | 71.7 (64.8, 77.1) | 75.0 (68.2, 81.8) | <0.001² |
| BMI | 29.1 (26.5, 33.2) | 26.8 (22.6, 32.6) | 27.8 (24.7, 31.0) | 0.008² |
| Frailty score | 0.23 (0.17, 0.29) n-miss = 0 | 0.14 (0.05, 0.27) n-miss = 0 | 0.10 (0.06, 0.18) n-miss = 20 | <0.001² |

¹Pearson’s Chi-Square, ²Kruskal-Wallis

TABLE 7

Characteristics of biologically older versus younger ovarian cancer participants

| | Non-frail | Frail | |
|---|---|---|---|
| | Number (%) or Median (Q1,Q3) | | p-value |
| n | 18 | 18 | |
| Age in years | 71.6 (65.4, 77.0) | 71.7 (65.5, 77.0) | 0.975¹ |
| Stage: IIIc | 15 (83.3%) | 15 (83.3%) | 1.000² |
| IV | 3 (16.7%) | 3 (16.7%) | |
| BMI | 23.0 (21.9, 26.9) | 31.6 (25.6, 34.2) | 0.001¹ |
| Frailty score | 0.05 (0.01, 0.07) | 0.27 (0.21, 0.31) | < 0.001¹ |

¹Kruskal-Wallis, ²Chi-Square

In unadjusted analyses, eight SASP factors, ACTIVIN A, CCL4, GDF15, IL6, IL15, OPN, TNFα, and TNFR1, were positively associated with the frailty index in any one of the three participant groups (Table 8; Model 1). GDF15 and OPN increased in association with the frailty index in all three participant groups and remained significant after adjusting for chronological age, BMI, and/or sex as potential confounding or effect modifying variables (Table 8). Similarly, after adjustment for age, BMI, and/or sex, TNFR1 was associated with a higher frailty index across all three groups. Increased CCL4 and TNFα were associated with advanced biological age in both surgical groups and remained significant after adjustment but was not significantly associated with frailty index in aged, non-surgical participants. IL15 was positively associated with frailty index in only the aortic stenosis and non-surgical participant groups, before and after adjustment. Using both unadjusted and adjusted models, ACTIVIN A and IL6 were positively associated with the frailty index in the non-surgical sample participants (Table 8).

TABLE 8

Circulating SASP factors associated with biological age

| | | Aortic Stenosis Ovarian Cancer | | | | | | | | Referent, 60-90y | | | |
|---|---|---|---|---|---|---|---|---|---|---|---|---|---|
| | | Model 1 | | Model 2 | | Model 1 | | Model 3 | | Model 1 | | Model 2 | |
| Protein | Alias | r-value | q-value | r-value | q-value | r-value | q-value | r-value | q-value | r-value | q-value | r-value | q-value |
| ACTIVIN A | INHBA | 0.034 | 0.743 | -0.066 | 0.352 | 0.226 | 0.301 | 0.237 | 0.026 | 0.414 | 0.001 | 0.345 | <0.001 |
| ADAMTS13 | VWFCP | 0.155 | 0.258 | 0.111 | 0.106 | -0.028 | 0.911 | -0.072 | 0.498 | -0.020 | 0.954 | 0.038 | 0.647 |
| CCL3 | MIP1A, SCYA3 | 0.187 | 0.172 | 0.187 | 0.005 | 0.298 | 0.238 | 0.316 | 0.003 | 0.037 | 0.933 | 0.001 | 0.990 |
| CCL4 | MIP1B, SCYA4 | 0.270 | 0.041 | 0.298 | <0.001 | 0.495 | 0.012 | 0.530 | <0.001 | 0.214 | 0.144 | 0.139 | 0.065 |
| CCL5 | RANTES, SCYA5 | 0.167 | 0.217 | 0.150 | 0.027 | 0.213 | 0.301 | 0.362 | 0.001 | -0.056 | 0.933 | -0.083 | 0.278 |
| CCL17 | TARC, SCYA17 | 0.085 | 0.503 | 0.083 | 0.240 | 0.216 | 0.301 | 0.284 | 0.005 | 0.039 | 0.933 | -0.031 | 0.682 |
| CCL22 | MDC, SCYA22 | 0.217 | 0.111 | 0.144 | 0.030 | 0.293 | 0.238 | 0.321 | 0.002 | 0.095 | 0.867 | 0.091 | 0.257 |
| FAS | APT1, TNFRSF6 | 0.129 | 0.362 | -0.026 | 0.712 | 0.269 | 0.270 | 0.322 | 0.003 | 0.076 | 0.912 | 0.029 | 0.682 |
| GDF15 | MIC1, NAG1, NRG1 | 0.304 | 0.034 | 0.313 | <0.001 | 0.490 | 0.012 | 0.344 | 0.001 | 0.369 | 0.002 | 0.333 | <0.001 |
| GDNF | | 0.081 | 0.504 | 0.047 | 0.499 | 0.220 | 0.301 | 0.106 | 0.311 | -0.069 | 0.912 | -0.103 | 0.221 |
| ICAM1 | CD54 | 0.132 | 0.355 | 0.070 | 0.338 | 0.274 | 0.238 | 0.124 | 0.237 | 0.098 | 0.867 | -0.047 | 0.550 |
| IL6 | | 0.093 | 0.468 | 0.207 | 0.001 | 0.043 | 0.866 | -0.203 | 0.054 | 0.301 | 0.016 | 0.303 | <0.001 |
| IL7 | | 0.113 | 0.409 | 0.061 | 0.390 | 0.231 | 0.301 | 0.311 | 0.003 | 0.014 | 0.954 | -0.089 | 0.257 |
| IL8 | CXCL8 | 0.166 | 0.217 | -0.005 | 0.926 | 0.361 | 0.118 | 0.159 | 0.138 | 0.043 | 0.933 | 0.029 | 0.682 |
| IL15 | | 0.291 | 0.034 | 0.296 | <0.001 | 0.173 | 0.401 | 0.156 | 0.139 | 0.279 | 0.028 | 0.280 | <0.001 |
| MMP2 | CLG4A | 0.056 | 0.633 | 0.057 | 0.407 | 0.243 | 0.295 | 0.363 | 0.001 | 0.011 | 0.954 | 0.063 | 0.415 |
| MMP9 | CLG4B | -0.046 | 0.682 | -0.146 | 0.029 | -0.191 | 0.358 | 0.190 | 0.071 | -0.015 | 0.954 | 0.124 | 0.112 |
| OPN | SPP1, PSEC0156 | 0.399 | 0.001 | 0.390 | <0.001 | 0.489 | 0.012 | 0.354 | 0.001 | 0.314 | 0.013 | 0.365 | <0.001 |
| PAI1 | SERPINE1, PLANH1 | 0.117 | 0.402 | 0.106 | 0.112 | 0.126 | 0.567 | 0.286 | 0.005 | -0.024 | 0.954 | 0.051 | 0.533 |
| PAI2 | SERPINEB2, PLANH2 | 0.188 | 0.172 | 0.136 | 0.039 | 0.063 | 0.805 | 0.214 | 0.043 | -0.038 | 0.933 | -0.080 | 0.283 |
| SOST | | -0.105 | 0.441 | -0.151 | 0.027 | 0.006 | 0.973 | 0.087 | 0.425 | 0.139 | 0.608 | 0.086 | 0.270 |
| TNFa | | 0.282 | 0.034 | 0.402 | < 0.001 | 0.499 | 0.012 | 0.413 | <0.001 | 0.090 | 0.867 | 0.067 | 0.396 |
| TNFR1 | | 0.246 | 0.058 | 0.263 | <0.001 | 0.488 | 0.012 | 0.450 | <0.001 | 0.375 | 0.002 | 0.359 | <0.001 |
| VEGFA | VPH | 0.249 | 0.058 | 0.231 | <0.001 | 0.287 | 0.238 | 0.308 | 0.004 | -0.006 | 0.956 | -0.017 | 0.807 |

Model 1: FDR-corrected spearman correlation of frailty score versus protein concentration.
Model 2: FDR-corrected spearman correlation of frailty score versus protein concentration adjusted for age, sex, and BMI.
Model 3: FDR-corrected spearman correlation of frailty score versus protein concentration adjusted for age and BMI.

### Circulating SASP Factors Are Associated With Adverse Post-Surgical Outcomes

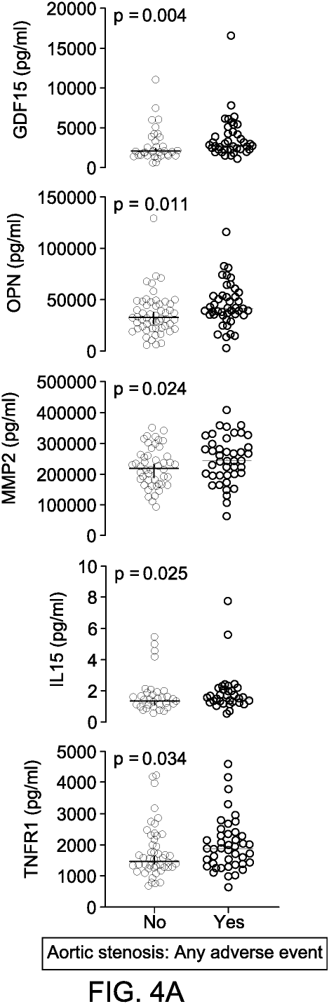

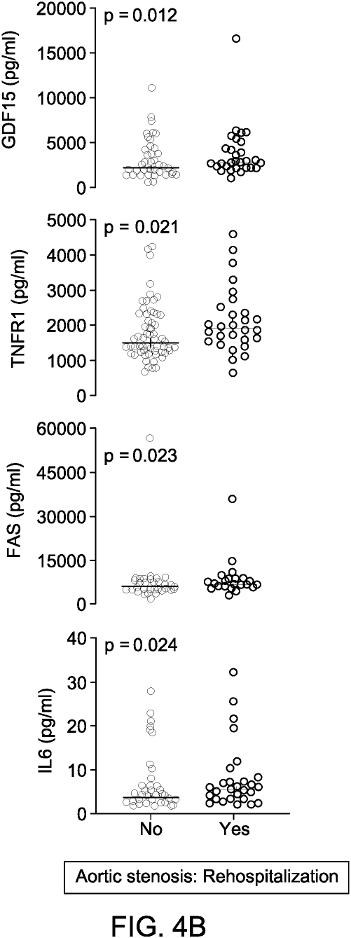

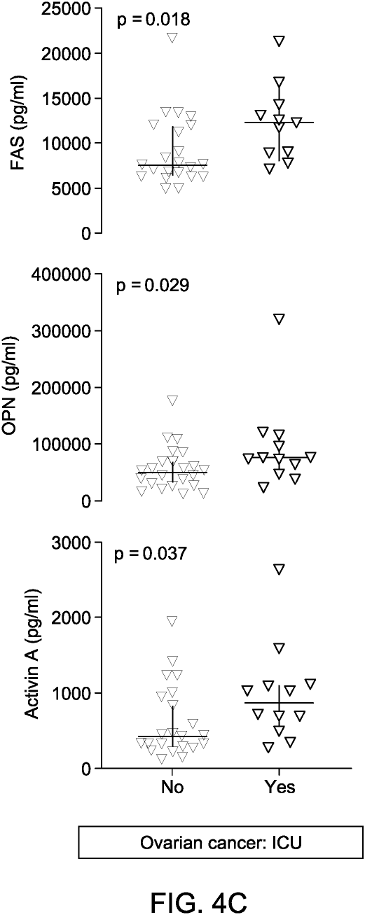

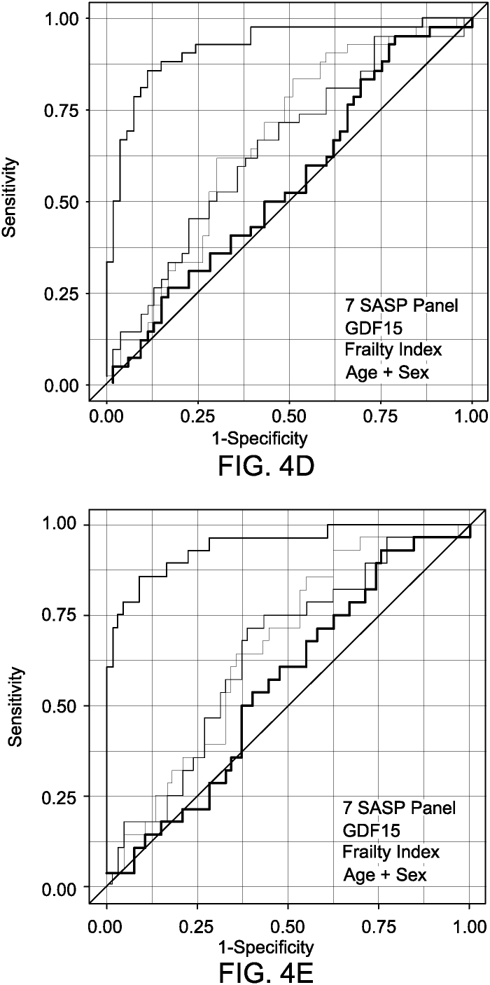

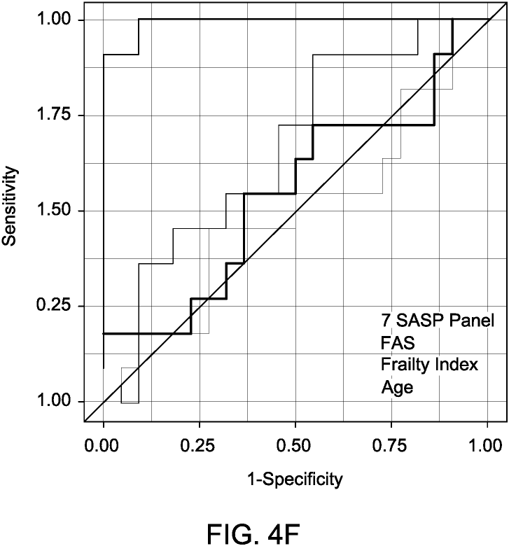

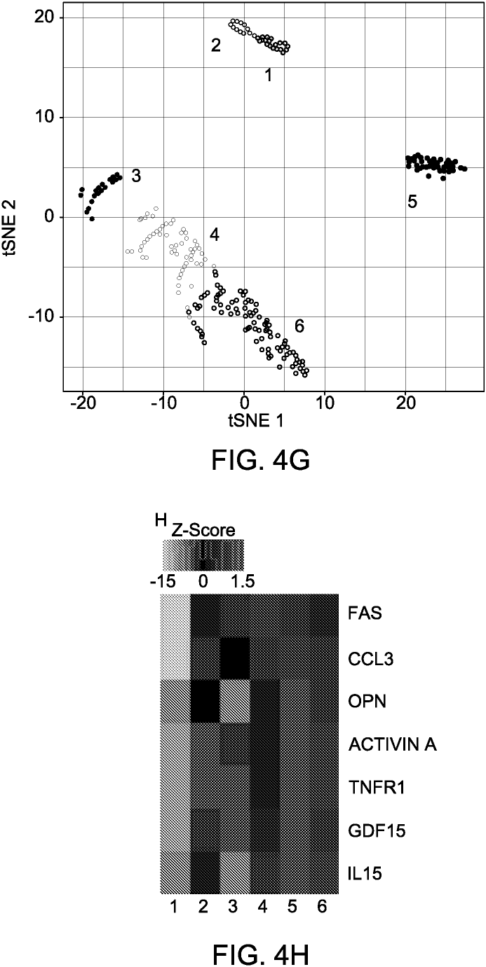

Relationships between pre-surgery circulating concentrations of SASP factors and adverse outcomes were examined in study participants who underwent surgery for severe aortic stenosis. Of 19 events assessed (myocardial infarction, new arrhythmia, new conduction abnormality, stroke, deep venous thrombosis, pulmonary emboli, pneumonia, plural effusion, new renal insufficiency, GI bleeding, new seizure disorder, significant hypotension, significant tachycardia, significant bradycardia, urinary tract infection, other infection, acute dementia, vascular complication, acute kidney injury), 42 individuals had at least one adverse event (43% of participants), and 55 individuals had no adverse event within 12 months of hospital discharge. Participants experiencing at least one adverse event were of similar chronological age but had higher median frailty scores compared to participants with no adverse events (frailty index = 0.26 vs. 0.20, p = 0.006). Median circulating GDF15, OPN, MMP2, IL 15, and TNFR1 concentrations were significantly higher in participants with at least one adverse event compared to participants without adverse events (FIG. 4A). As predictors of risk of an adverse event within 12 months of surgery, the receiver operating characteristic area under the curve (ROC AUC) for GDF15 was 0.66, while the ROC AUCs for frailty score and age + sex were 0.65 and 0.56, respectively (FIG. 4D).

Rehospitalization within 12 months of hospital discharge was assessed as a separate variable from any adverse event. Twenty-eight of the 97 participants undergoing surgery for severe aortic stenosis (29%) were rehospitalized within one year of surgical discharge. Participants who were rehospitalized at least once were of similar chronological age, but had advanced biological age compared to participants that were not (frailty index = 0.27 vs. 0.21, p = 0.012). Median circulating GDF15, TNFR1, FAS, and IL6 concentrations were significantly higher among the rehospitalized versus non-rehospitalized participants (FIG. 4B). The rehospitalization predictive ability of pre-surgery circulating GDF15, TNFR1, or IL6 levels was equivalent to that of biological age (GDF15 ROC AUC = 0.66; TNFR1 ROC AUC = 0.66; IL6 ROC AUC = 0.66; frailty index ROC AUC = 0.66) and potentially greater than the predictive ability of age + sex (age + sex ROC AUC = 0.56) (FIG. 4E).

In study participants who underwent surgery for ovarian cancer, the most common adverse event experienced was admission to the ICU within 30 days of surgery (12 of 36 total participants (33%)). Eight of the 12 individuals who were admitted to the ICU were frail cases and four were non-frail controls, representing a non-significant relationship between frailty and ICU admission (p = 0.157). Median circulating levels of FAS, OPN, and ACTIVIN A prior to surgery were significantly higher among participants who were admitted to the ICU within 30 days of surgery, relative to those who were not (FIG. 4C). FAS, OPN, and ACTIVIN A were identified as the most robust candidate biomarker predictors of risk of an adverse event within 12 months of surgery, with potentially higher predictive power relative to either chronological age or biological age alone (FAS ROC AUC = 0.76; OPN ROC AUC = 0.74; ACTIVIN A ROC AUC = 0.74; age ROC AUC = 0.50; frailty index ROC AUC = 0.63) (FIG. 4F).

Gradient boosting machine (GBM) modeling was next used to identify a single panel including SASP proteins capable of predicting adverse outcomes better than age or single factor across the distinct aortic stenosis and ovarian cancer patient samples. A seven protein panel including GDF15, FAS, OPN, TNFR1, ACTIVIN A, CCL3, and IL15 was consistently able to predict adverse events in both surgical populations more robustly than a single protein, biological age, or chronological age + sex. Specifically, the ROC AUCs for discriminating risk of any adverse event or rehospitalization within 12 months of surgery for severe aortic stenosis were 0.84 and 0.81, respectively (FIGS. 4D-E). The ROC AUC for discriminating risk of admission to the ICU within 30 days of surgery for ovarian cancer was 0.85 (FIG. 4F).

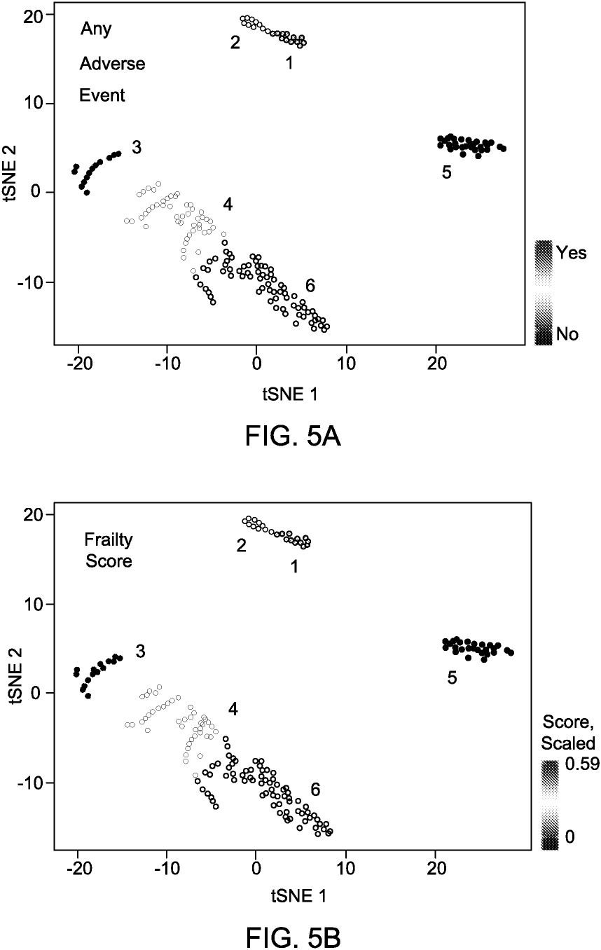

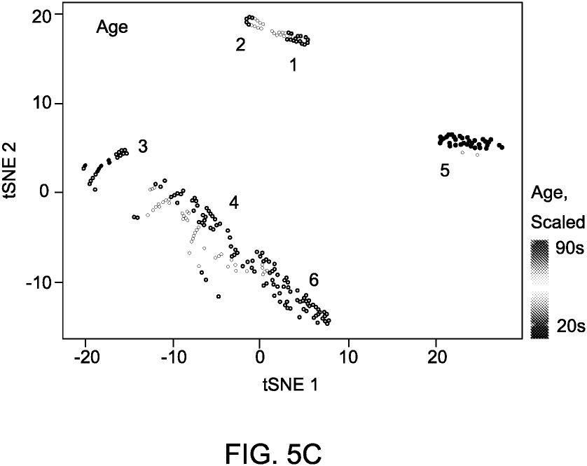

To explore the GBM-identified panel through another approach, t-distributed stochastic neighbor embedding (tSNE) projection was utilized to generate phenotypic participant clusters for circulating SASP factor comparisons. All participant samples in which frailty status was ascertained and all SASP proteins were measured were applied to this analysis (n = 343). The presence of any post-operative adverse event (any adverse event or rehospitalized within 12 months of surgery for the aortic stenosis group, ICU admission within 30 days of surgery for the ovarian cancer group), frailty score, and age were used as cluster definition variables (FIG. 5), rendering six clusters (FIG. 4G). Cluster one and two were comprised of non-frail participants with no adverse events, with cluster one chronologically younger and cluster two chronologically older. Cluster three and four were comprised of participants with low to moderate frailty and no adverse events. Cluster five was comprised of participants with higher frailty scores and adverse events, and cluster six was comprised of participants with higher frailty scores and no adverse events. Scaled comparison of the GBM-identified seven candidate biomarker panel (FIG. 4H) revealed distinct profiles of SASP factor concentrations per cluster, with higher levels of GBM-identified SASP factors demarcating older, more frail adults that had an adverse event following surgery (cluster five) from those that did not (cluster six).

As shown herein, distinct senescent cell types secrete SASP polypeptides, with senescent endothelial cells, preadipocytes, and fibroblasts producing a more robust SASP polypeptides, as compared to senescent epithelial cells and myoblasts.

Taken together, these results also demonstrate that senescent cells secrete increased levels of GDF15 polypeptides, FAS polypeptides, OPN polypeptides, TNFR1 polypeptides, ACTIVIN A polypeptides, CCL3 polypeptides, and IL15 polypeptides. For example, increased levels of these polypeptide can be detected in a blood sample obtained from a mammal to identify the presence of senescent cells within the mammal. For example, increased levels of these polypeptides can be detected in a blood sample obtained from a mammal to identify the mammal as being more likely to experience frailty and/or adverse post-surgery outcomes.

### METHODS

### Cell Culture Experiments

Human fibroblasts (IMR90; American Type Culture Collection (ATCC, Manassas, VA, USA)) were cultured in Dulbecco’s Modified Eagle Medium (DMEM) containing 10% Fetal Bovine Serum (FBS) and Penicillin-Streptomycin-Glutamine (Gibco). Primary human preadipocytes isolated from three healthy kidney donors were cultured in Minimum Essential Medium a (a-MEM) containing 10% FBS and Penicillin-Streptomycin-Glutamine. Human Umbilical Vein Endothelial Cells (HLTVEC; Lonza, Basel, Switzerland) were cultured in EGM-2 BulletKit (Lonza). Human epithelial cells (ARPE-19; ATCC) were cultured in DMEM/F12 containing 10% FBS and Penicillin-Streptomycin-Glutamine. Human myoblasts derived from healthy donors (Cook MyoSite, Pittsburgh, PA, USA) were cultured in skeletal muscle cell growth medium (Promocell, Heidelberg, Germany). Cells were exposed to sham conditions or, to induce senescence, 10 Gy radiation using a RS2000 X-Ray Irradiator (RAD Source Technologies, Suwanee, GA, USA). Fibroblasts, preadipocytes, epithelial cells, and myoblasts were then cultured for 21 days and HUVECs were cultured for 7 days prior to collection. Cells were provided fresh media every three days. After the indicated time, senescence was confirmed by staining for senescence-associated β-galactosidase (SA-β-Gal) and real-time PCR analysis of cyclin-dependent kinase inhibitor (p16 and p21) and SASP gene expression. For SA-(β-Gal staining, cells were fixed in phosphate-buffered 4% paraformaldehyde for 10 minutes at room temperature. Cells were then washed twice with PBS and incubated overnight (16-18 hours) in SA-β-Gal staining solution (1 mg/mL X-Gal, 40 mM citric acid/sodium phosphate buffer pH 6.0, 5 mM potassium ferrocyanide, 5 mM potassium ferricyanide, 150 mM sodium chloride and 2 mM magnesium chloride) at 37° C. on a shaker and in the dark. Cells were then washed twice with PBS and nuclei stained with Hoechst dye for 5 minutes. Fluorescence microscopy (Eclipse Ti, Nikon, Japan) was used for imaging. Images were taken under bright field for SA-β-Gal staining and the same field under blue fluorescence channel for nuclear staining. Conditioned media from cultured fibroblasts, preadipocytes, myoblasts and epithelial cells were obtained by exposing cells to RMPI 1640 containing 1 mM sodium pyruvate, 2 mM glutamine, minimum essential medium (MEM) vitamins, MEM nonessential amino acids, and Penicillin-Streptomycin. Non-senescent and senescent cells were washed three times with PBS and then cultured for 24 hours before media were collected. For HUVECs, cells were washed three times with PBS and then cultured in EBM-2 medium with 0.5% FBS for 24 hours before media were collected. Conditioned media were filtered through 0.22 µm filter prior to analysis. Cells were trypsinized, counted, and harvested in Trizol (Invitrogen, Invitrogen, Carlsbad, CA, USA) for RNA isolation according to manufacturer’s instructions. RNA concentration was assessed by Nanodrop (Thermo Fisher Scientific, Waltham, MA, USA). cDNA was synthesized using M-MLV reverse transcriptase (Invitrogen), and real-time PCR was performed with PerfeCTa FastMix II (QuantaBio, Beverly, MA, USA) and the Applied Biosystems StepOne Plus Real-Time PCR system (Applied Biosystems, Foster City, CA, USA). Gene expression was analyzed by delta-delta CT method and normalized to the reference gene, TATA-Box Binding Protein (TBP). The primers and probes used are listed in Table 9.

TABLE 9

Primers and probes for real-time PCR

| | | | |
|---|---|---|---|
| Gene | Primers and probes catalog number or sequence | SEQ ID NO: | Source |
| TBP | Hs.PT.58.20792004 | | IDT |
| P16 | Forward primer: 5′ CCAACGCACCGAATAGTTACG 3′ | 1 | IDT |
| | Reverse primer: 5′ GCGCTGCCCATCATCATG 3′ | 2 | |
| | Probe: 5′ FAM - CCTGGATCGGCCTCCGAC - ZEN / IBFQ 3′ | 3 | |
| P21 | Hs.PT.58.40874346.g | | IDT |
| IL6 | Hs.PT.58.40226675 | | IDT |

### Assessment of the SASP in Biological Fluids and Cell Culture Media

The concentration of ADAMTS13, CCL3, CCL4, CCL5, CCL17, CCL22, FAS, GDF15, GDNF, ICAM1, IL15, IL6, IL7, IL8, MMP2, MMP9, OPN, PAI1, SOST, TNFR1, TNFa, and VEGFA in conditioned media from non-senescent and senescent cells and EDTA plasma were quantified using commercially available multiplex magnetic bead immunoassays (R&D Sytems, Minneapolis, MN) based on Luminex® xMAP multianalyte profiling platform and analyzed on MAGPIX® System (Merck Millipore). All assays were performed according to the manufacturer’s protocols. ACTIVIN A concentration was determined by a Quantikine ELISA Kit (R&D Systems) according to the manufacturer’s instructions. PAI2 concentration was determined by an ELISA Kit (Cloud-Clone Corp., Katy, TX, USA) according to the manufacturer’s instructions. For all proteins, more than 80% of the samples were within the detectable range. Undetectable targets were assigned a value of half of the lowest value; the number and percentage of imputed samples per target are summarized in Table 10.

TABLE 10

Summary of imputed values

| Protein | Alias | Number Imputed Values | Percentage Imputed of All Samples per Target |
|---|---|---|---|
| ACTIVIN A | INHBA | 0 | 0% |
| ADAMTS13 | VWFCP | 0 | 0% |
| CCL3 | MIP1A, SCYA3 | 40 | 10% |
| CCL4 | MIP1B, SCYA4 | 8 | 2% |
| CCL5 | RANTES, SCYA5 | 0 | 0% |
| CCL17 | TARC, SCYA17 | 1 | 0% |
| CCL22 | MDC, SCYA22 | 0 | 0% |
| FAS | APT1, TNFRSF6 | 0 | 0% |
| GDF15 | MIC1, NAG1, NRG1 | 5 | 1% |
| GDNF | ATF | 52 | 13% |
| ICAM1 | CD54 | 0 | 0% |
| IL6 | IFNB2 | 0 | 0% |
| IL7 | | 0 | 0% |
| IL8 | CXCL8 | 0 | 0% |
| IL15 | | 0 | 0% |
| MMP2 | CLG4A | 0 | 0% |
| MMP9 | CLG4B | 0 | 0% |
| OPN | SPP1, PSEC0156 | 16 | 4% |
| PAI1 | SERPINE1, PLANH1 | 0 | 0% |
| PAI2 | SERPINEB2, PLANH2 | 0 | 0% |
| SOST | DAND6 | 0 | 0% |
| TNFa | TNFSF2 | 1 | 0% |
| TNFR1 | CD 120a | 0 | 0% |
| VEGFA | VPH | 1 | 0% |

### Participant Samples

Biobank sample: The Mayo Clinic biobank is comprised of residents of Olmsted County, Minnesota (n = 56,964) who donate biological specimens and provide risk factor data, access to clinical data obtained from the medical record, and consent to participate in approved research. The Mayo Clinic Biobank started enrolment in April 2009. Participants are predominantly white (95%). F or the present study, archived plasma samples were requested from 280 participants between 20 and 90 years of age (20 women and 20 men per decade). Participants with a history of cancer, other than breast cancer and melanoma, prior to the age of 50, or autoimmune diseases (e.g., rheumatoid arthritis, lupus), and women with BMI < 18.5 or > 40.0 kg/m² and men with BMI < 18.5 or > 35.0 kg/m² were excluded. Samples of 13 participants were of insufficient volume for SASP factor analysis, resulting in n = 267.

Aortic stenosis sample: This sample included women and men scheduled for surgical or transcatheter aortic valve replacement. Demographic characteristics and medical history, including previous surgical events and diagnoses, were ascertained by interview, physical exam, and electronic medical record review at baseline. Adverse post-operative events were recorded 1, 3, 6, and/or 12 months post discharge from the hospital. For assessment of any adverse event within 12 months of discharge, the following outcomes were considered: myocardial infarction, new arrhythmia, new conduction abnormality, stroke, deep venous thrombosis, pulmonary emboli, pneumonia, plural effusion, new renal insufficiency, GI bleeding, new seizure disorder, significant hypotension, significant tachycardia, significant bradycardia, urinary tract infection, other infection, acute dementia, vascular complication, or acute kidney injury. Rehospitalization within 12 months of discharge was considered as a separate adverse event.

Ovarian cancer sample: This sample included patients who underwent primary cytoreductive surgery for stage IIIC or IV ovarian cancer, fallopian tube, or primary peritoneal cancer. Exclusion criteria included patients who received neoadjuvant chemotherapy, patients undergoing palliative or diagnostic surgeries only, patients without frailty index available, and patients who denied access to their medical record. All patients had a surgical resection to < 1 cm of residual disease and all had a BMI < 40 kg/m². Cases were defined as patients having a frailty index >0.15. Cases were matched by age (within 3 years) and cancer stage to non-frail controls.

### Frailty Index

The frailty index was calculated using a combination of comorbidities and patient-provided activities of daily living (ADL) variables abstracted from the medical record. The index reflects the percent of variables that a given subject experienced. The comorbidity variables assessed were myocardial infarct, diabetes, peripheral vascular disease, chronic obstructive pulmonary disease, hypertension, hyperlipidemia, BMI (underweight/obese = 1 point, overweight = 0.5 points), anemia, cerebrovascular disease, dementia, peptic ulcer, hemiplegia/paraplegia, renal disease, moderate/severe liver disease, rheumatologic disease, any malignancy, metastatic solid tumor, and depression. The ADL variables assessed were difficulty preparing meals, difficulty feeding oneself, difficulty dressing, difficulty using the toilet, difficulty housekeeping, difficulty climbing stairs, difficulty bathing, difficulty walking, difficulty using transportation, difficulty getting in and out of bed, difficulty taking medications/managing medications, dependent on assistive devices (cane, wheelchair, braces, walker, others), and dependent on device for breathing (CPAP, nasal oxygen).

### Statistical Analyses

Two-tailed t-tests were used to compare cell culture data. Descriptive statistics (percentages or medians/25th/75th percentiles and means/SD) were used to summarize the characteristics among patient cohorts. Comparisons between the groups were performed using chi-square and Wilcoxon rank-sum tests. Spearman correlation coefficients were used to summarize the relationship between the proteins and age. Univariate logistic regression models were fit predicting adverse events using the candidate biomarker, age, sex, and frailty score variables; ROC AUCs were estimated from these models. GBM, a machine learning technique, was used to create a multivariable prediction model using the GBM package available in the R software environment. This technique involves combining information from multiple decision trees that are iteratively built in such a way that each iteration focuses increasingly on the portions of the data that are most ill-fitting. The number of trees included in the model (number of iterations), the depth of the trees and the size of the shrinkage parameter were determined by 5-fold cross-validation. AUC values from the GBM models were optimism corrected using an internal validation bootstrap process, since external data to validate the AUC values were not utilized. As part of the model results, variables are ranked in importance indicating relative contribution to the models. tSNE clustering and phenograph analysis was performed using the cytofkit package. All other analyses were performed using SAS version 9.4 (SAS Institute, Cary, NC, USA), R 3.4.2 (R Foundation for Statistical Computing, Vienna, Austria), R 3.6.0, or GraphPad Prism 8.1.2 (San Diego, CA).

### Example 2: The Senescence-Associated Secretome as an Indicator of Survival After Ovarian Cancer

### Study Aim

Assess gradient boosting models predicting survival based on senescence biomarkers and known clinical predictors.
Evaluating the ability of 30+ biomarkers (including Activin A, FAS, osteopontin, GDF15, II,15, and TNFR1) to, when combined, identify patients likely to have better or worse outcomes following surgery related to ovarian cancer treatment.

Cohort Details are as shown in Table 11:

TABLE 11

| | | | |
|---|---|---|---|
| | Frail 0.15-1) (N==56) | Not Frail(<0.15) (N=224) | p value |
| Year of Surgery | | | 0.601¹ |
| Median | 2006.0 | 2006.0 | |
| Q1, Q3 | 2004.0, 2011.2 | 2004.0, 2014.0 | |
| Age at surgery (years) | | | 0.050¹ |
| Median | 60.7 | 63.6 | |
| QI, Q3 | 59.8, 74.0 | 55.4, 70.9 | |
| BMI | | | < 0.001¹ |
| Median | 31.1 | 25.5 | |
| Q1, Q3 | 26.5, 36.6 | 22.8, 29.6 | |
| BMI group | | | < 0.001² |
| Underwt (<18.5) | 0(0.0%) | 4(1.8%) | |
| Normal (18.5-24.9) | 10 (17.9%) | 98 (43.8%) | |
| Overwt (25.0-29.9) | 14 (25.0%) | 72 (32.1%) | |
| Obesity I (30.0-34.9) | 13 (23.2%) | 31 (13.8%) | |
| Obesity II (35.9-39.9) | 12 (21.4%) | 9 (4.0%) | |
| Obesity III (40.0+) | 7 (12.5%) | 10 (4.5%) | |
| ASA level 3+ | | | < 0.001³ |
| No | 16 (28.6%) | 137 (61.2%) | |
| Yes | 40 (71.4%) | 87 (38.8%) | |
| Preoperative albumin g/dL) | | | 0.003¹ |
| N-Miss | 21 | 90 | |
| Median | 3.7 | 4.0 | |
| Q1, Q3 | 3.4, 4.0 | 3.6, 4.4 | |
| Preoperative albumin (g/dL) | | | 0.176³ |
| IMPreop albumin <3 g/dL | 3 (5.4%) | 3 (1.3%) | |
| 1= Preop albumin >= 3 g/dL | 32 (57.1%) | 131 (58.5%) | |
| 2= Preop albumin missing | 21 (37.5%) | 90 (40.2%) | |
| FIGO Grade | | | 0.302³ |
| 1-2 | 1 (1.8%) | 11 (4.9%) | |
| 3 | 55 (98.2%) | 213 (95.1%) | |
| FIGO Stage | | | 0.616³ |
| 9=IIIc | 42 (75.0%) | 175 (78.1%) | |
| 10= IV | 14 (25.0%) | 49 (21.9%) | |
| Serous histology | | | 0.517³ |
| No | 8 (14.3%) | 25 (11.2%) | |
| Yes | 48 (85.7%) | 199 (88.8%) | |
| Surgical complexity | | | 0.074³ |
| Low | 14 (25.0%) | 33 (14.7%) | |
| Intermediate | 32 (57.1%) | 124 (55.4%) | |
| High | 10 (17.9%) | 67 (29.9%) | |
| Residual disease | | | 0.117³ |
| Microscopic | 20 (35.7%) | 107 (47.8%) | |
| Yes: measurable | 22 (39.3%) | 84 (37.5%) | |
| Yes: suboptimal/extensive | 14 (25.0%) | 33 (14.7%) | |
| Ascites | | | 1.000³ |
| No | 17 (30.4%) | 68 (30.4%) | |
| Yes | 39 (69.6%) | 156 (60.6%) | |
| rwscore | | | <0.001¹ |

### Statistical Methods

GBM, a machine learning technique, was used to create the model predicting survival. Analysis was run using the generalized boosted model package (gbm3) available in the R software environment. This technique involves combining information from multiple decision trees and is sometimes referred to as ensemble learning. The trees are built in such a way that each iteration focuses increasingly on the portions of the data that was most ill-fitting. The chief advantage of this method is that it naturally incorporates interactions between variables, is not as susceptible to extreme values and handles missing values without the need to impute data.

The number of trees included in the model (number of iterations, tried 100-1000), the depth of the trees (1=no interactions, 2=2-way interactions, 3=3-way interactions, 4=4-way interactions) and the size of the shrinkage parameter (0.001, 0.005, 0.01) were determined by repeated 5-fold cross-validation maximizing the discrimination ability of the model. This is the recommended approach to prevent overfitting of the model. Discrimination, the ability of a risk score to accurately rank individuals from low to high risk, was assessed by calculating Harrell’s C-statistic. Assessment of the C-statistic using the original data can lead to overly optimistic results. For large datasets the data is often split into training and testing subsets, however that is not appropriate for smaller datasets such as is available here. Instead, some sort of internal validation process is needed. This internal validation process needs to include all the steps used for building the model including the selection of the shrinkage parameter, number of trees, and depth of the trees. The approach used here is as follows:

1. Develop the GBM model using repeated cross-validation to select the 3 parameters (shrinkage, number of trees, depth of the tree).

2. Create an outer cross-validation process:
Divide the data into 5 even groups
Use the full process from step 1 on ⅘ths of the data to obtain a best set of the 3 parameters and use that model to obtain predictions on the ⅕th of the data not used in the modeling process.
Repeat, each time holding out a different 5th of the data

3. Combine the predictions from each cross-validation (⅕ + ⅕ + ⅕ + ⅕ + ⅕) and use these predictions to estimate the C-statistic.

This model was fit using:
age, BMI, and all the biomarkers

The unadjusted c-statistic is 0.66 and the adjusted c-statistic is 0.59. The best set of parameters for the model included a tree depth of 1, a shrinkage of 0.001, and 700 trees. The top variables include:

| | |
|---|---|
| TNF RI | 25.01 |
| GDF-15 | 22.10 |
| Fas | 17.43 |
| IL-6 | 7.88 |
| BMI | 7.76 |
| TNF RII | 7.56 |
| MMP-7 | 3.07 |
| Activin A | 1.74 |
| PAI-1 | 1.56 |
| Age at surgery | 1.20 |
| MMP-2 | 0.92 |
| IL-15 | 0.73 |
| SOST | 0.60 |
| Osteopontin | 0.52 |
| PARC | 0.50 |
| MMP-9 | 0.36 |
| Eotaxin | 0.32 |
| MPO | 0.15 |
| MCP-1 | 0.11 |
| STC-1 | 0.09 |
| MDC | 0.09 |
| RANTES | 0.08 |
| IL-7 | 0.08 |
| IL-8 | 0.07 |
| GRO alpha | 0.07 |

### Plot Results

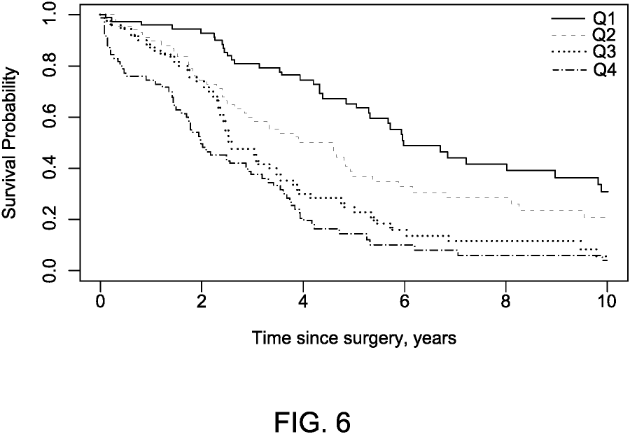

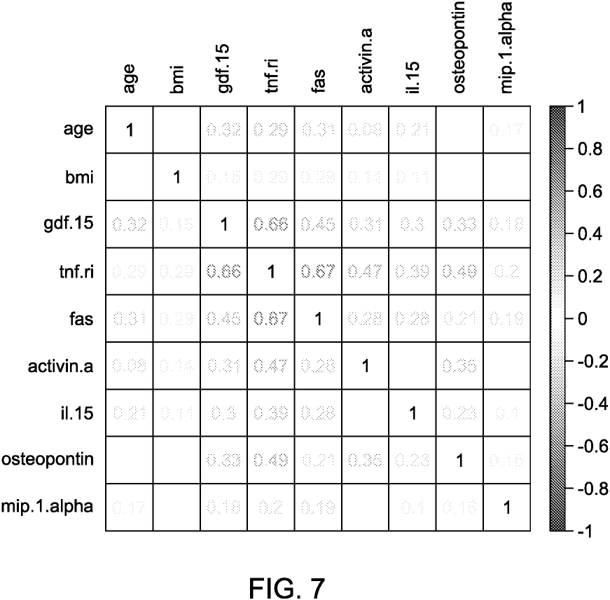

As one way to examine the results, the predicted values from the GBM model were plotted against some of the top predictors (FIG. 6). The plot survival curve was stratified by quartiles of the predicted values (Ql=lowest predicted values, Q4=highest predicted values).

### Example 3: The Senescence-Associated Secretome as an Indicator of Survival After Ovarian Cancer

Additional GBM summaries were generated using various combinations of SASP biomarkers.

### Run With 7 Markers (Adj for Age, Bmi)

A model was fit using:
age, bmi
7 biomarkers: GDF 15, FAS, OPN, TNFRI, ACTIVIN A, MIP lA, and II,15.

The unadjusted c-statistic is 0.67 and the adjusted c-statistic is 0.61. The best set of parameters for the model included a tree depth of 1, a shrinkage of 0.005, and 300 trees. The unadjusted c-statistic values, stratified by age (<60, 60-69, 70+) are: 0.65, 0.68, and 0.66 respectively. The top variables include:

TABLE 12

| labels | rel_inf |
|---|---|
| TNF RI | 26.92 |
| GDF-15 | 22.01 |
| Fas | 20.28 |
| BMI | 14.54 |
| Age at surgery (years) | 7.67 |
| Osteopontin | 3.93 |
| Activin A | 2.97 |
| IL-15 | 1.18 |
| MIP-I alpha | 0.53 |

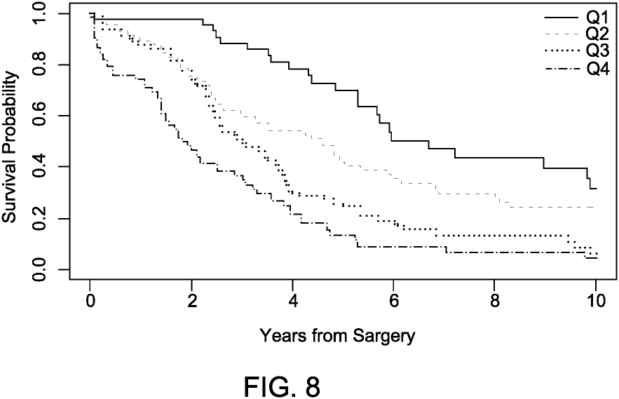

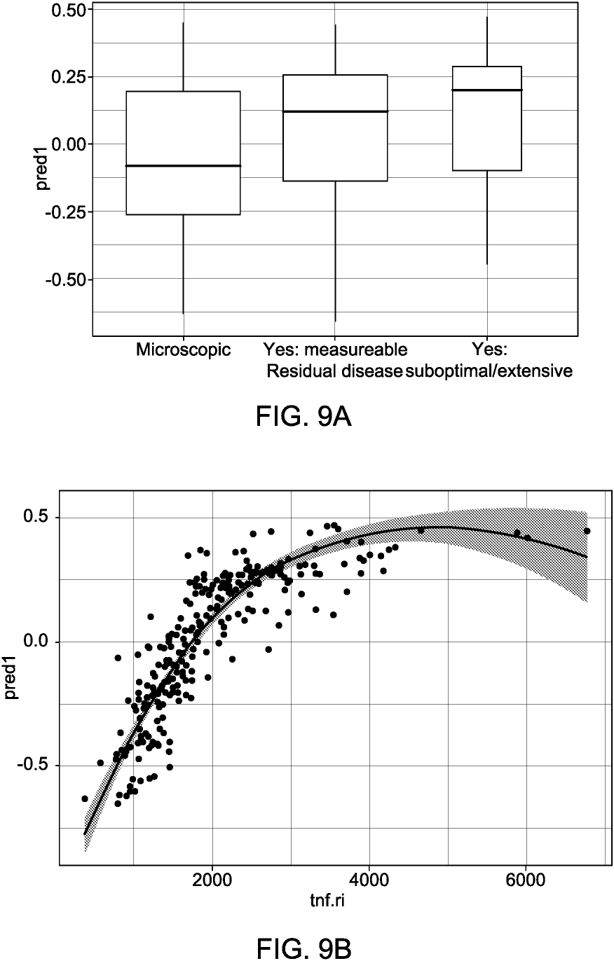

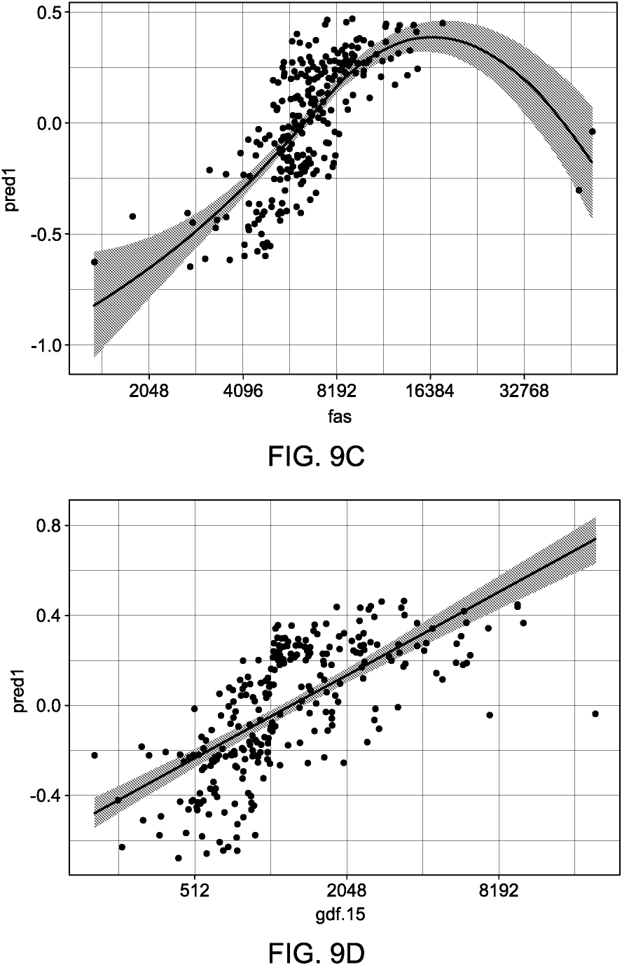

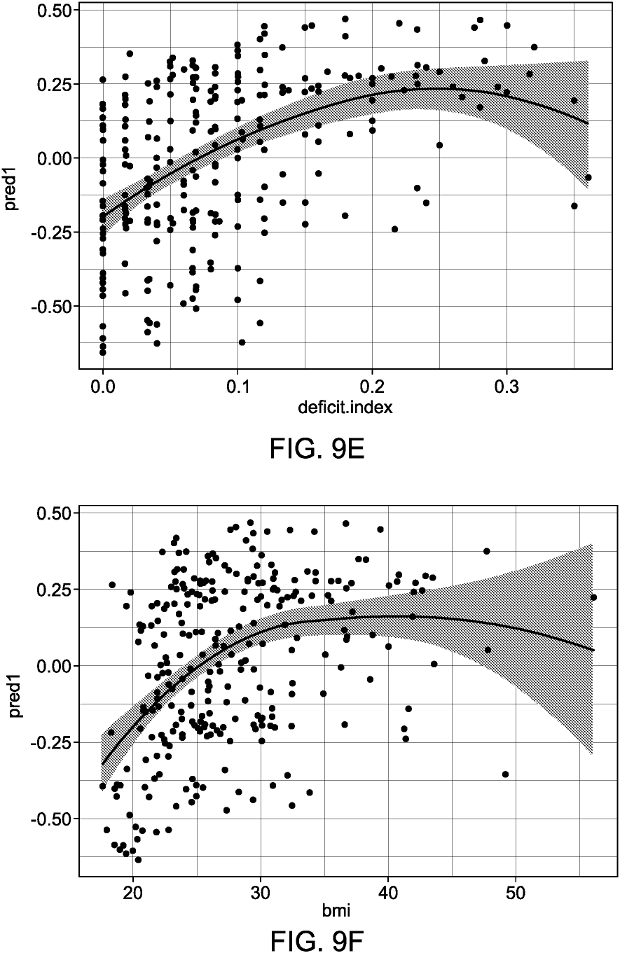

As one way to examine the results, the predicted values from the GBM model were looked at and plotted those against some of the top predictors. Resulting plots are shown in FIG. 8 and FIGS. 9A-9F.

### Run with 7 Markers

This model was fit using:
7 biomarkers: GDF 15, FAS, OPN, TNFRI, ACTIVIN A, MIP1A, and IL15

The unadjusted c-statistic is 0.65 and the adjusted c-statistic is 0.6. The best set of parameters for the model included a tree depth of 1, a shrinkage of 0.001, and 100 trees. The top variables include:

TABLE 13

| labels | rel_inf |
|---|---|
| TNF RI | 49.82 |
| Fas | 26.39 |
| GDF-15 | 23.40 |
| Osteopontin | 0.38 |

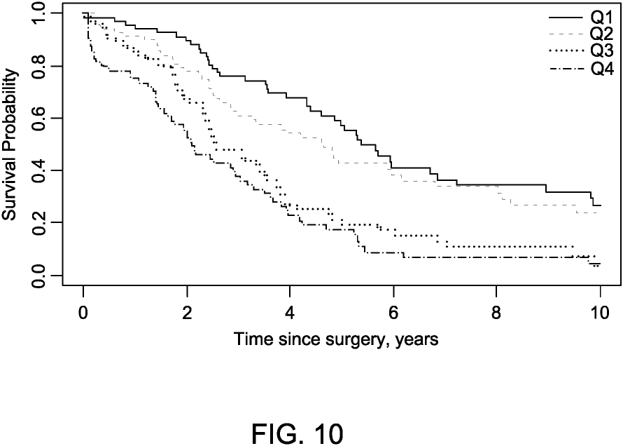

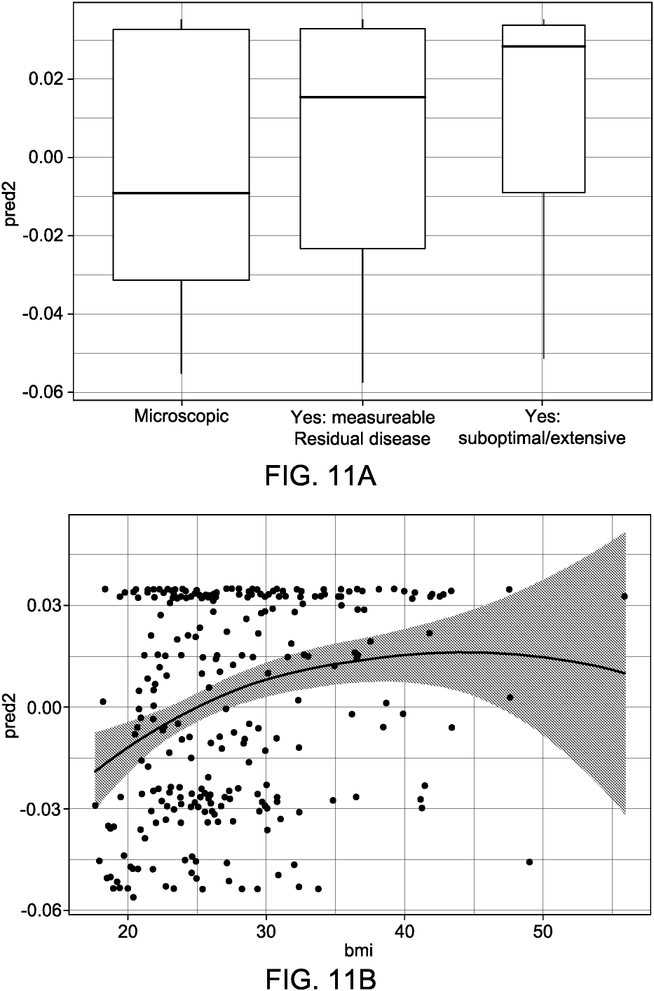

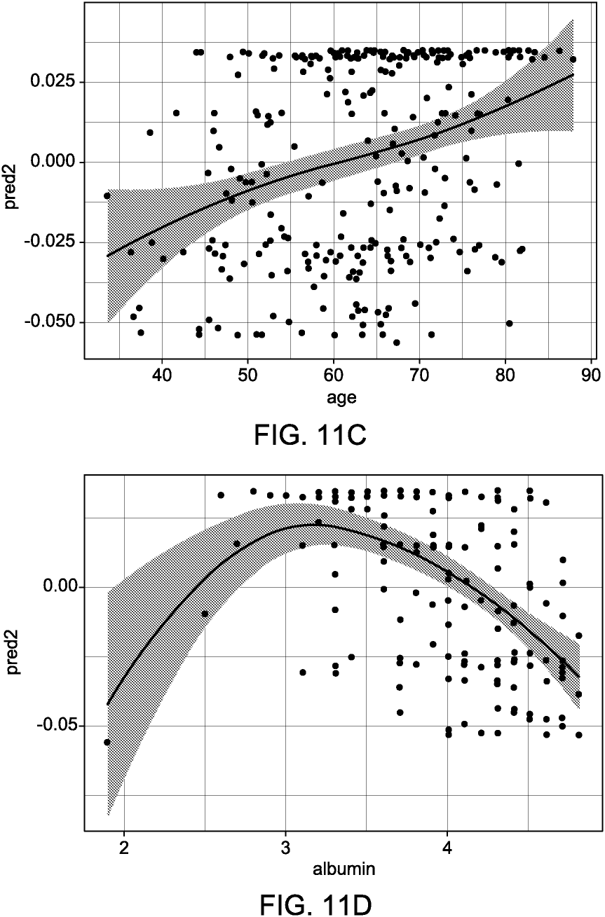

As one way to examine the results, the predicted values from the GBM model were looked at and plotted those against some of the top predictors. Resulting plots are shown in FIG. 10 and FIGS. 11A-11D.

### Run With Only Age and Bmi

This model was fit using:
age, bmi

The unadjusted c-statistic is 0.62 and the adjusted c-statistic is 0.53. The best set of parameters for the model included a tree depth of 1, a shrinkage of 0.01, and 200 trees. The top variables include:

TABLE 14

| labels | rel_inf |
|---|---|
| BMI | 54.33 |
| Age at surgery (years) | 45.67 |

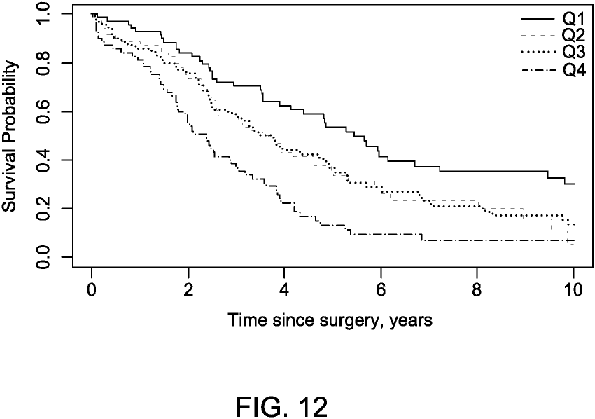

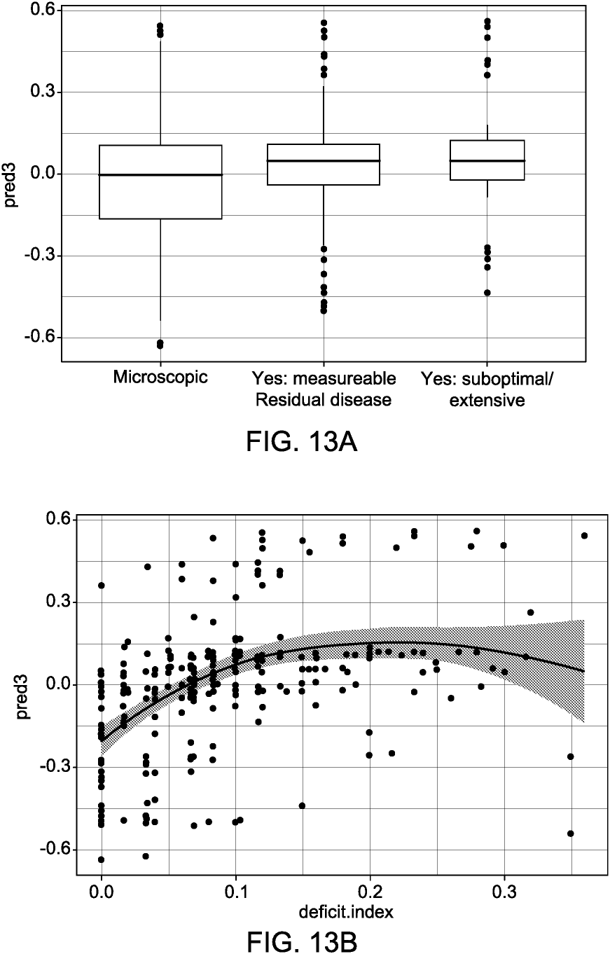

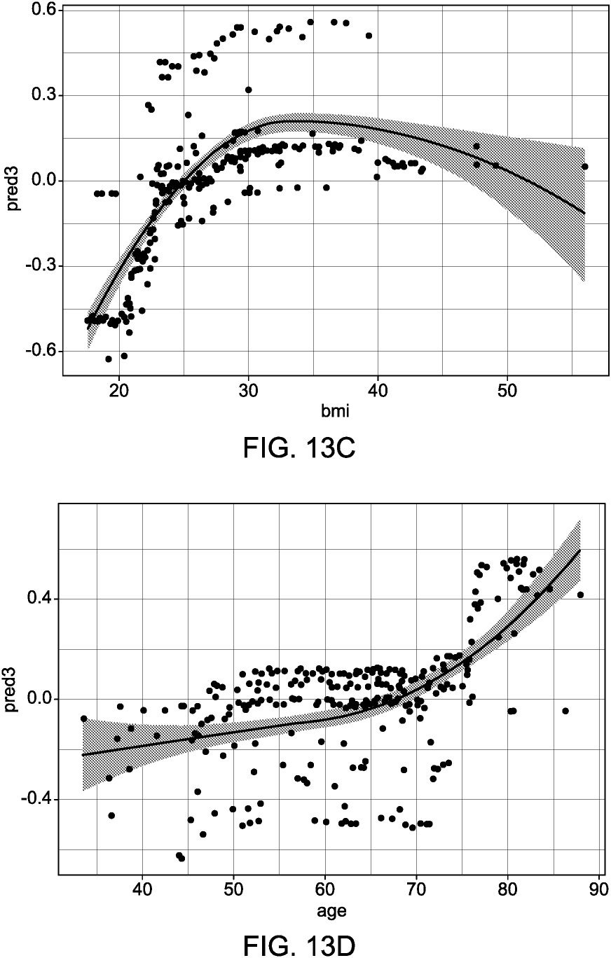

As one way to examine the results, the predicted values from the GBM model were looked at and plotted those against some of the top predictors. Resulting plots are shown in FIG. 12 and FIGS. 13A-13D.

### Run With 4 Markers

This model was fit using:
4 biomarkers: GDF 15, TNFRI, FAS, ACTIVIN A

The unadjusted c-statistic is 0.65 and the adjusted c-statistic is 0.61. The best set of parameters for the model included a tree depth of 1, a shrinkage of 0.001, and 700 trees. The top variables include:

TABLE 15

| labels | rel_inf |
|---|---|
| TNF RI | 39.84 |
| GDF-15 | 32.26 |
| Fas | 26.63 |
| IL-15 | 1.27 |

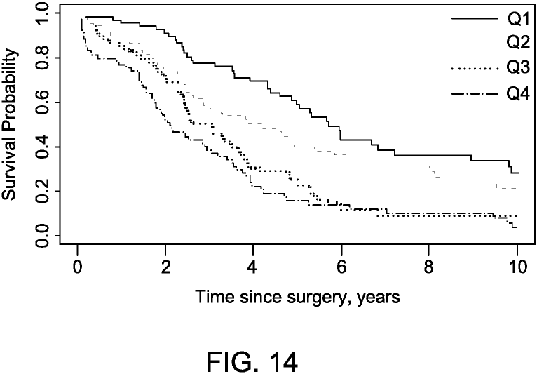

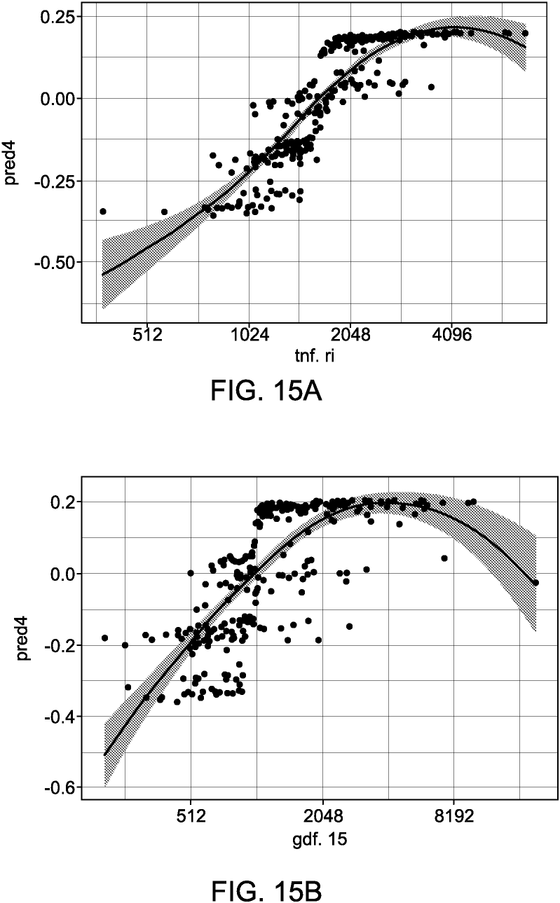

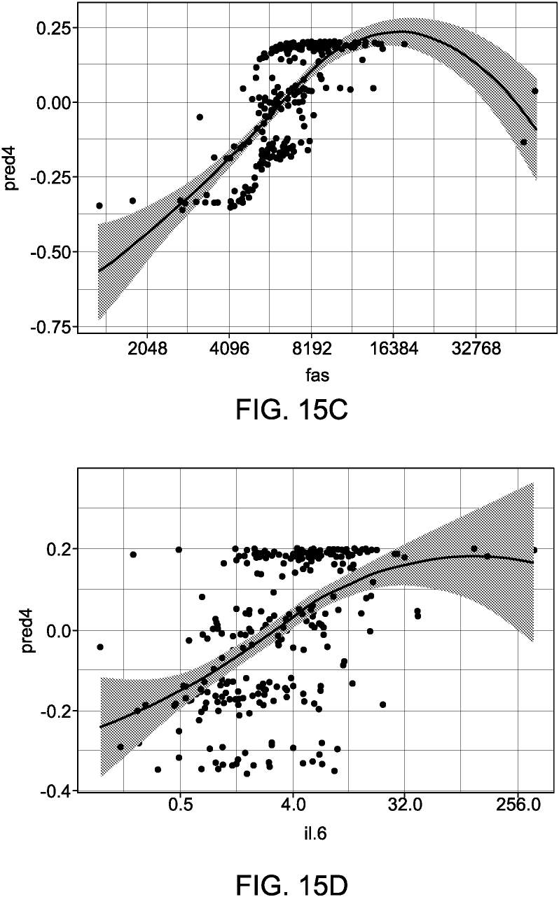

As one way to examine the results, the predicted values from the GBM model were looked at and plotted those against some of the top predictors. Resulting plots are shown in FIG. 14 and FIGS. 15A-15D.

### Run With 5 Markers

This model was fit using:
5 biomarkers: GDF 15, TNFRI, FAS, ACTIVIN A, and IL15

The unadjusted c-statistic is 0.65 and the adjusted c-statistic is 0.61. The best set of parameters for the model included a tree depth of 1, a shrinkage of 0.001, and 600 trees. The unadjusted c-statistic values, stratified by age (<60, 60-69, 70+) are: 0.64, 0.65, and 0.64 respectively. The top variables include:

TABLE 16

| labels | rel_inf |
|---|---|
| TNFRI | 37.59 |
| GDF-15 | 32.49 |
| Fas | 26.04 |
| Activin A | 2.95 |
| IL-15 | 0.93 |

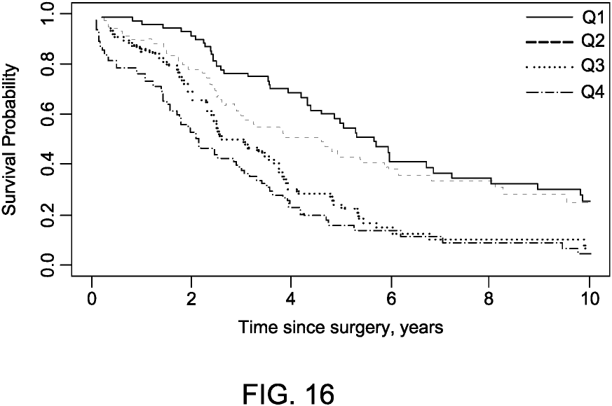

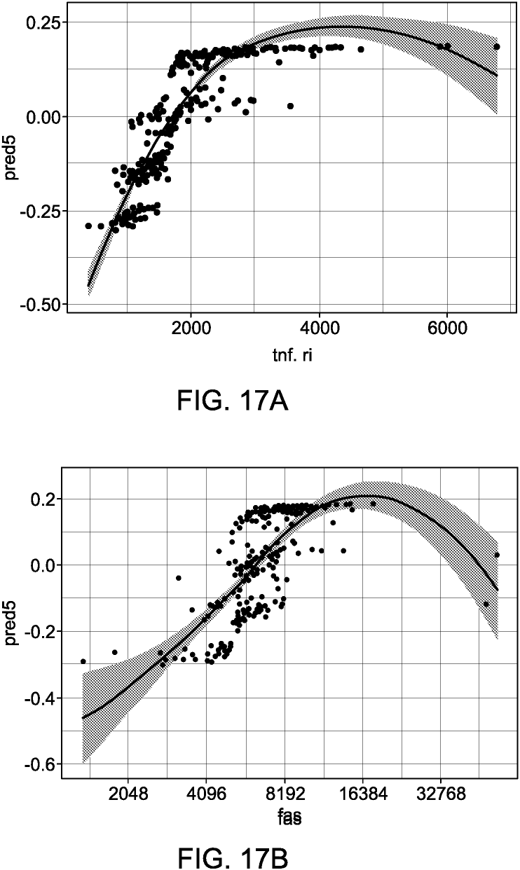

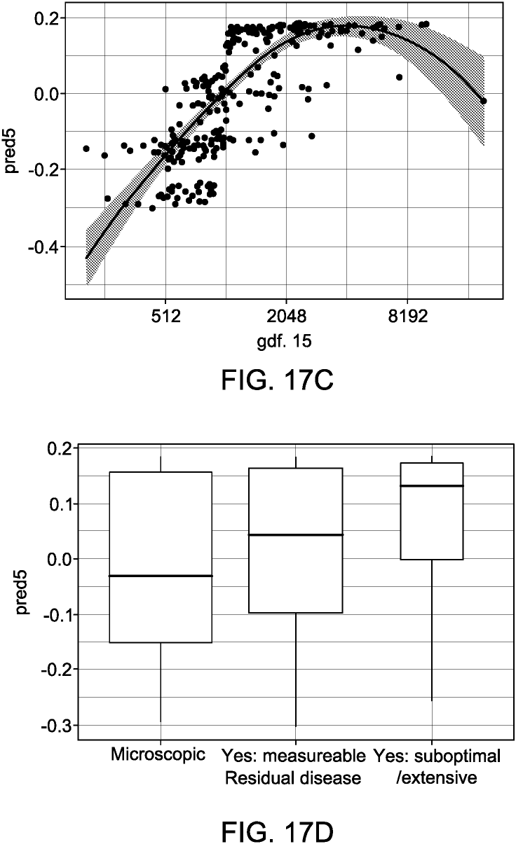

As one way to examine the results, the predicted values from the GBM model were looked at and plotted those against some of the top predictors. Resulting plots are shown in FIG. 16 and FIGS. 17A-17D.

### Run With 6 Markers

This model was fit using:
6 biomarkers: GDF15, TNFRI, FAS, ACTIVIN A, IL15, and OPN

The unadjusted c-statistic is 0.65 and the adjusted c-statistic is 0.6. The best set of parameters for the model included a tree depth of 1, a shrinkage of 0.001, and 800 trees. The unadjusted c-statistic values, stratified by age (<60, 60-69, 70+) are: 0.64, 0.65, and 0.64 respectively. The top variables include:

TABLE 17

| labels | rel_inf |
|---|---|
| TNF RI | 37.44 |
| GDF-15 | 29.77 |
| Fas | 26.28 |
| Activin A | 3.09 |
| Oateopontin | 1.90 |
| IL-15 | 1.52 |

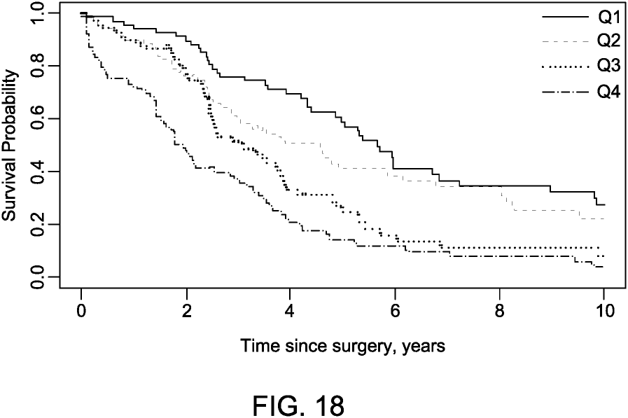

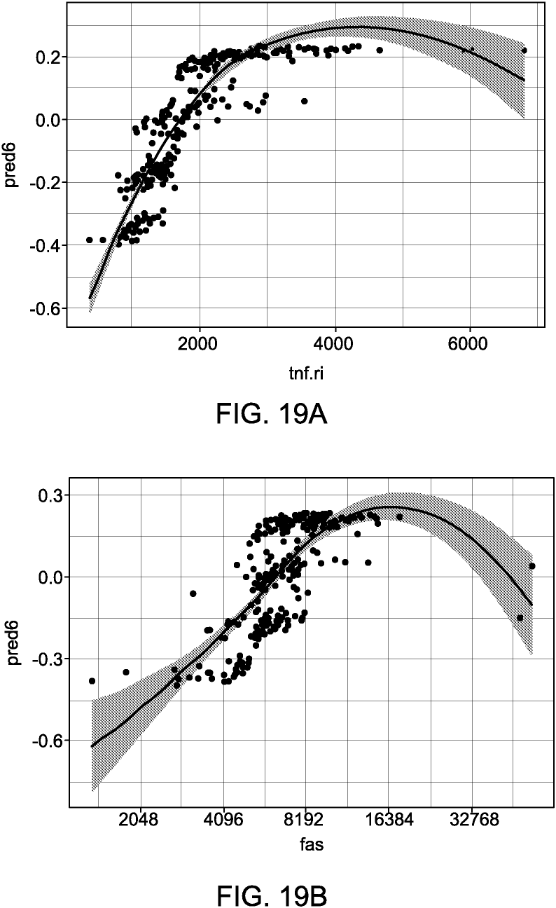

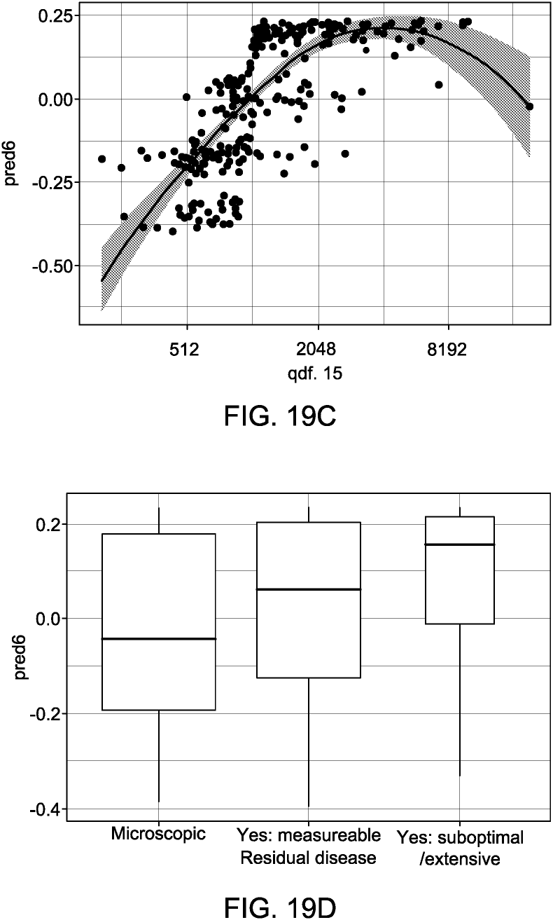

As one way to examine the results, the predicted values from the GBM model were looked at and plotted those against some of the top predictors. Resulting plots are shown in FIG. 18 and FIGS. 19A-19D.

### Penalized Cox Models

Since the top GBM models all appear to be using trees with just 1 split (i.e., no interactions), penalized Cox Cox models were looked at using all the variables used in the GBM analysis. A log transformation has been applied to the biomarkers divided by the sd of the log of X. Age is per 10 year increase and BMI is per a 5 point change. 3 scenarios were run:
age, bmi, and 7 biomarkers
7 biomarkers: GDF 15, FAS, OPN, TNFRI, ACTIVIN A, MIP1A, and IL15
7 biomarkers, forced model to keep IL-15

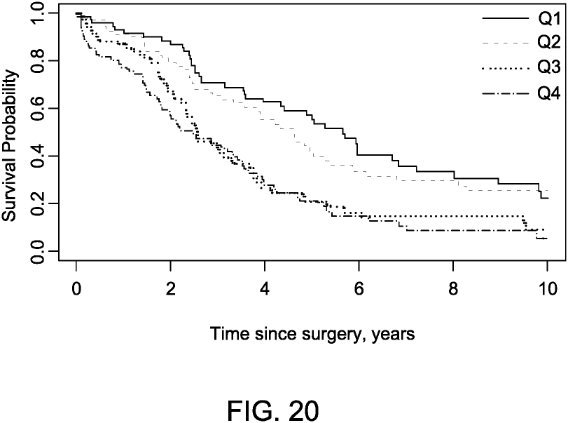

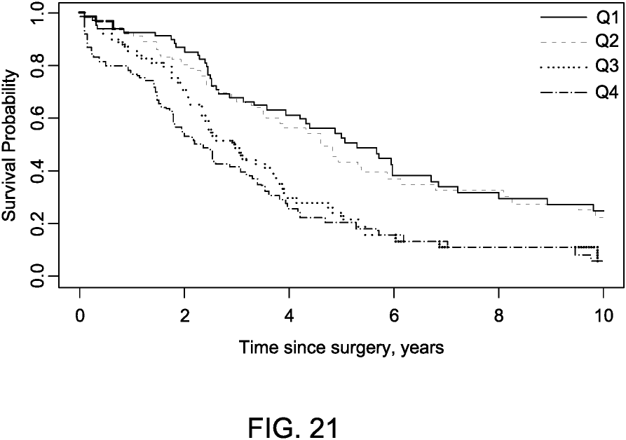

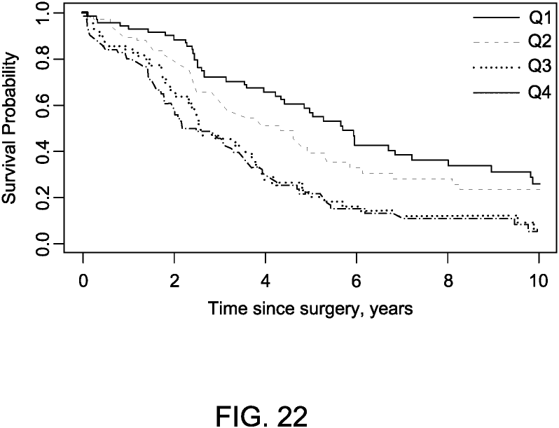

The “alpha” was varied, allowing the proportion of the L 1 and L2 penalty to range (0,0.25,0.5,0.75,1). L1-penalty tends to drops variables and the L2 penalty tends to keeps all the variables and shrinks the values. Using a value between 0 and 1 is called the “elastic net”. A representative c-statistic from each scenario is shown in FIG. 20, FIG. 21, and FIG. 22. A setting of alpha=0.25 was selected, and penalizing all 7 markers versus keeping IL-15 was examined.

TABLE 18

Maximum c-statistic for different alpha values and different sets of covariates

| alpha | allvars | markers7 | keep.iI15 |
|---|---|---|---|
| 0.00 | 0.62 | 0.62 | 0.61 |
| 0.25 | 0.62 | 0.63 | 0.62 |
| 0.60 | 0.62 | 0.63 | 0.60 |
| 0.75 | 0.63 | 0.60 | 0.61 |
| 1.00 | 0.62 | 0.61 | 0.61 |

TABLE 19

Hazard ratios for variables in the model

| | | | |
|---|---|---|---|
| | allvars | markers7 | keep.iI15 |
| age.10 | 1.016 | NA | NA |
| bmf.5 | 1.026 | NA | NA |
| gdf.15 | 1.083 | — | 1.008 |
| tnf.ri | 1.164 | 1.032 | 1.175 |
| fas | 1.039 | — | 1.050 |
| activin.a | 1.011 | — | 1.02 |
| IL.15 | — | — | 1.005 |
| osteopontin | — | — | — |
| mip.1.alpha | — | — | — |

The dashed lines in Table 19 indicate the variable was shrunk to zero, and NA indicates the variable wasn’t included in the model. The hazard ratios are per 1 SD change. GDF15, FAS, TNFRI, ACTIVIN A, and IL15appear to be strong predictors.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Protein concentration pg/ml/million cells

| | ACTIVIN A | ADAMTS13 | CCL3 | CCL4 | CCL5 | CCL17 | CCL22 | FAS |
|---|---|---|---|---|---|---|---|---|
| HUVEC C1 | 54.43 | 7997.85 | 102.34 | 156.27 | 3.88 | 93.35 | 32.68 | 21.76 |
| HUVEC C2 | 47.77 | 7071.12 | 69.17 | 136.96 | 3.54 | 78.76 | 28.45 | 19.43 |
| HUVEC C3 | 53.67 | 8558.22 | 88.26 | 154.08 | 4.10 | 90.74 | 32.51 | 22.30 |
| HUVEC SnC1 | 856.12 | 69992.62 | 608.17 | 960.14 | 30.81 | 648.19 | 226.75 | 173.72 |
| HUVEC SnC2 | 975.63 | 78541.98 | 581.47 | 1092.99 | 33.86 | 718.08 | 260.09 | 199.27 |
| HUVEC SnC3 | 949.93 | 87142.37 | 682.54 | 1170.17 | 36.77 | 762.95 | 281.14 | 230.36 |
| Preadipocytes C1 | 97.78 | 7351.64 | 345.86 | 424.75 | 4.77 | 88.98 | 115.54 | 66.91 |
| Preadipocytes C2 | 73.04 | 3783.93 | 201.90 | 301.47 | 2.45 | 91.60 | 59.47 | 54.80 |
| Preadipocytes C3 | 279.30 | 5198.13 | 277.36 | 459.37 | 3.37 | 125.84 | 128.78 | 87.37 |
| Preadipocytes SnC1 | 2823.62 | 72390.97 | 350.87 | 1678.38 | 38.49 | 819.76 | 368.55 | 254.26 |
| Preadipocytes SnC2 | 4690.39 | 67958.87 | 352.24 | 1554.21 | 31.00 | 769.57 | 327.10 | 260.93 |
| Preadipocytes SnC3 | 5172.35 | 68304.04 | 332.83 | 1384.66 | 27.62 | 685.62 | 291.41 | 222.55 |
| Fibroblasts C1 | 153.16 | 5189.39 | 244.13 | 301.69 | 3.36 | 125.62 | 81.55 | 31.48 |
| Fibroblasts C2 | 0.16 | 5189.39 | 80.75 | 301.69 | 3.36 | 62.81 | 40.78 | 19.81 |
| Fibroblasts C3 | 0.16 | 5189.39 | 205.72 | 301.69 | 3.36 | 62.81 | 40.78 | 23.68 |
| Fibroblasts SnC1 | 182.89 | 10378.78 | 276.89 | 571.00 | 3.36 | 215.86 | 152.79 | 107.44 |
| Fibroblasts SnC2 | 213.09 | 10378.78 | 183.23 | 690.75 | 3.36 | 289.32 | 152.79 | 127.77 |
| Fibroblasts SnC3 | 274.58 | 20354.73 | 244.13 | 717.34 | 3.36 | 289.32 | 170.49 | 123.69 |
| Epithelial C1 | 67.00 | 10093.14 | 107.07 | 196.49 | 3.55 | 103.53 | 42.58 | 47.73 |
| Epithelial C2 | 83.26 | 10360.70 | 120.38 | 218.64 | 3.99 | 116.40 | 45.19 | 46.79 |
| Epithelial C3 | 64.58 | 11601.05 | 73.10 | 212.48 | 3.96 | 109.73 | 45.13 | 49.94 |
| Epithelial SnC1 | 217.14 | 30187.94 | 529.43 | 676.54 | 11.16 | 352.66 | 142.93 | 140.17 |
| Epithelial SnC2 | 402.66 | 51660.77 | 505.81 | 1118.31 | 18.83 | 544.05 | 229.94 | 264.13 |
| Epithelial SnC3 | 190.14 | 44548.05 | 546.03 | 815.92 | 17.39 | 471.90 | 173.29 | 207.97 |
| Myoblasts C1 | 98.33 | 10265.18 | 260.95 | 246.11 | 0.86 | 103.08 | 60.74 | 25.35 |
| Myoblasts C2 | 0.31 | 11822.66 | 253.25 | 377.95 | 1.98 | 145.32 | 80.89 | 35.79 |
| Myoblasts C3 | 0.10 | 9518.65 | 216.78 | 274.47 | 2.65 | 135.90 | 59.12 | 28.04 |
| Myoblasts SnC1 | 74.69 | 6383.09 | 128.17 | 164.65 | 1.33 | 82.01 | 36.39 | 21.65 |
| Myoblasts SnC2 | 86.43 | 6982.45 | 44.00 | 129.14 | 3.49 | 70.06 | 26.17 | 34.16 |
| Myoblasts SnC3 | 89.91 | 10767.67 | 116.95 | 232.94 | 4.35 | 134.19 | 49.81 | 43.31 |
| OVCAR8 CM1 | 0.03 | 372.01 | 65.79 | 21.49 | 0.30 | 7.74 | 9.22 | 1.98 |
| OVCAR8 CM2 | 0.03 | 2005.01 | 50.97 | 64.10 | 0.59 | 28.77 | 13.72 | 5.23 |
| OVCAR8 CM3 | 0.02 | 653.48 | 62.75 | 35.95 | 0.27 | 13.59 | 9.62 | 2.72 |

Protein concentration pg/ml/million cells

| | | | | | | | | |
|---|---|---|---|---|---|---|---|---|
| | GDF15 | GDNF | ICAM1 | IL6 | IL7 | IL8 | IL15 | MMP2 |
| | 126.95 | 0.76 | 1719.71 | 13.30 | 3.95 | 26.09 | 3.46 | 5764.24 |
| HUVEC C1 | 123.41 | 0.74 | 1504.00 | 12.54 | 3.37 | 24.36 | 2.94 | 4983.55 |
| HUVEC C2 | 144.55 | 0.92 | 1765.54 | 14.01 | 3.79 | 26.84 | 3.36 | 5545.57 |
| HUVEC C3 | 4809.40 | 7.24 | 11965.22 | 81.16 | 27.39 | 1043.51 | 25.35 | 51089.58 |
| HUVEC SnC1 | 5894.52 | 7.74 | 13949.63 | 87.44 | 30.50 | 1155.25 | 29.08 | 57020.29 |
| HUVEC SnC2 | 5866.17 | 10.01 | 15412.34 | 97.24 | 32.90 | 1368.17 | 29.21 | 59941.16 |
| HUVEC SnC3 | 37.83 | 1.18 | 884.59 | 21.84 | 2.68 | 2.45 | 0.15 | 3715.45 |
| Preadipocytes C1 | 26.67 | 0.61 | 910.60 | 14.76 | 2.53 | 4.91 | 0.16 | 4250.96 |
| Preadipocytes C2 | 30.24 | 0.83 | 2085.17 | 35.50 | 3.48 | 8.24 | 0.22 | 4854.07 |
| Preadipocytes C3 | 270.63 | 12.45 | 17627.80 | 2347.57 | 37.68 | 129.81 | 29.12 | 25085.92 |
| Preadipocytes SnC1 | 247.02 | 9.54 | 16024.91 | 1967.01 | 31.69 | 64.72 | 22.19 | 28134.70 |
| Preadipocytes SnC2 | 135.84 | 8.50 | 14276.73 | 1378.21 | 29.88 | 34.39 | 22.84 | 17163.40 |
| Preadipocytes SnC3 | 20.88 | 0.83 | 2591.52 | 0.37 | 1.89 | 3.49 | 2.69 | 2179.17 |
| Fibroblasts C1 | 16.22 | 0.09 | 2591.52 | 0.18 | 0.10 | 2.02 | 2.69 | 883.71 |
| Fibroblasts C2 | 17.39 | 0.83 | 2591.52 | 0.18 | 0.20 | 3.19 | 2.69 | 1826.09 |
| Fibroblasts C3 | 178.45 | 2.36 | 4634.62 | 9.45 | 6.51 | 21.69 | 3.82 | 6300.57 |
| Fibroblasts SnC1 | 229.54 | 2.36 | 5183.04 | 16.90 | 7.99 | 54.65 | 5.39 | 6745.86 |
| Fibroblasts SnC2 | 231.87 | 2.36 | 6218.85 | 15.42 | 9.45 | 31.02 | 5.39 | 9233.84 |
| Fibroblasts SnC3 | 6.79 | 0.92 | 2173.01 | 2.69 | 4.62 | 55.17 | 4.04 | 1944.67 |
| Epithelial C1 | 7.63 | 1.16 | 2383.96 | 2.29 | 5.42 | 43.38 | 4.34 | 1657.13 |
| Epithelial C2 | 7.38 | 1.10 | 2467.50 | 2.33 | 5.11 | 48.82 | 4.46 | 1599.15 |
| Epithelial C3 | 28.37 | 2.92 | 7512.23 | 5.32 | 14.65 | 87.68 | 12.74 | 8693.67 |
| Epithelial SnC1 | 44.71 | 5.15 | 12474.32 | 12.94 | 27.02 | 233.24 | 21.15 | 18192.23 |
| Epithelial SnC2 | 36.60 | 5.15 | 9681.96 | 9.84 | 21.20 | 211.52 | 18.55 | 14533.98 |
| Epithelial SnC3 | 31.61 | 1.12 | 2034.40 | 0.27 | 3.37 | 1.03 | 1.92 | 7425.88 |
| Myoblasts C1 | 12.87 | 1.61 | 3143.11 | 0.31 | 5.64 | 3.99 | 4.57 | 6339.31 |
| Myoblasts C2 | 9.09 | 1.02 | 2572.10 | 0.60 | 5.05 | 4.09 | 3.61 | 3589.42 |
| Myoblasts C3 | 88.94 | 0.55 | 1575.45 | 0.73 | 2.90 | 2.12 | 1.95 | 5841.65 |
| Myoblasts SnC1 | 72.86 | 0.81 | 1452.46 | 1.37 | 2.95 | 8.25 | 2.46 | 11650.46 |
| Myoblasts SnC2 | 90.90 | 1.34 | 2539.69 | 2.43 | 5.23 | 7.77 | 4.08 | 13069.34 |
| Myoblasts SnC3 | 3.99 | 0.06 | 200.69 | 0.05 | 0.14 | 0.12 | 0.02 | 9.09 |
| OVCAR8 CM1 | 7.90 | 0.22 | 595.98 | 0.11 | 1.06 | 0.24 | 0.68 | 26.18 |
| OVCAR8 CM2 | 7.52 | 0.15 | 326.34 | 0.05 | 0.41 | 0.15 | 0.24 | 18.19 |
| OVCAR8 CM3 | | | | | | | | |

Protein concentration pg/ml/million cells

| | MMP9 | OPN | PAI1 | PAI2 | SOST | TNFα | INFR1 | VEGFA |
|---|---|---|---|---|---|---|---|---|
| HUVEC C1 | 18.88 | 2295.35 | 770949 | 11.74 | 2.49 | 2.34 | 17.75 | 3.89 |
| HUVEC C2 | 16.43 | 1973.56 | 164969 | 10.95 | 1.94 | 2.07 | 15.20 | 3.44 |
| HUVEC C3 | 19.96 | 2438.88 | 156078 | 13.08 | 2.48 | 2.41 | 17.38 | 4.00 |
| HUVEC SnC1 | 133.43 | 51952.09 | 8808587 | 99.80 | 18.97 | 16.44 | 170.34 | 31.08 |
| HUVEC SnC2 | 151.09 | 58894.22 | 10103968 | 107.09 | 23.49 | 19.13 | 195.39 | 35.65 |
| HUVEC SnC3 | 173.05 | 64520.33 | 10735466 | 112.59 | 23.12 | 20.32 | 205.65 | 36.12 |
| Preadipocytes C1 | 68.72 | 5998.25 | 3467 | 0.03 | 5.98 | 1.04 | 42.98 | 42.78 |
| Preadipocytes C2 | 33.44 | 3087.33 | 5928 | 0.01 | 3.08 | 1.78 | 24.02 | 29.27 |
| Preadipocytes C3 | 51.25 | 4630.31 | 6226 | 0.02 | 4.23 | 1.47 | 32.99 | 36.52 |
| Preadipocytes SnC1 | 174.60 | 14319.38 | 133459 | 14.71 | 16.89 | 23.72 | 152.30 | 567.51 |
| Preadipocytes SnC2 | 149.14 | 12531.53 | 327226 | 48.05 | 5.69 | 15.86 | 139.44 | 416.92 |
| Preadipocytes SnC3 | 146.03 | 11774.20 | 343526 | 40.43 | 14.12 | 17.95 | 111.66 | 233.92 |
| Fibroblasts C1 | 40.58 | 6487.27 | 905 | 0.02 | 2.38 | 0.34 | 32.94 | 32.72 |
| Fibroblasts C2 | 32.72 | 3838.89 | 241 | 0.02 | 2.38 | 0.17 | 22.56 | 21.10 |
| Fibroblasts C3 | 40.58 | 4234.06 | 451 | 0.02 | 2.38 | 0.17 | 27.75 | 27.64 |
| Fibroblasts SnC1 | 59.15 | 6847.81 | 4095 | 0.02 | 2.38 | 2.45 | 75.92 | 59.10 |
| Fibroblasts SnC2 | 77.94 | 6487.27 | 6200 | 0.02 | 2.38 | 5.14 | 85.06 | 68.25 |
| Fibroblasts SnC3 | 67.18 | 6847.81 | 5704 | 0.02 | 2.38 | 6.00 | 90.28 | 65.98 |
| Epithelial C1 | 23.40 | 2705.17 | 70705 | 12.10 | 2.60 | 2.70 | 17.02 | 77.82 |
| Epithelial C2 | 25.40 | 2741.06 | 81451 | 13.72 | 2.44 | 2.97 | 18.28 | 75.40 |
| Epithelial C3 | 27.39 | 2820.33 | 89558 | 11.31 | 3.40 | 2.80 | 19.66 | 74.31 |
| Epithelial SnC1 | 79.17 | 9347.02 | 153292 | 27.36 | 6.24 | 8.35 | 71.59 | 699.20 |
| Epithelial SnC2 | 133.48 | 16150.44 | 319390 | 62.98 | 12.16 | 16.08 | 119.03 | 1168.02 |
| Epithelial SnC3 | 101.86 | 15718.13 | 445542 | 57.68 | 13.88 | 12.14 | 89.53 | 918.51 |
| Myoblasts C1 | 25.85 | 2311.33 | 2683 | 0.01 | 0.85 | 2.14 | 32.17 | 12.99 |
| Myoblasts C2 | 36.42 | 2662.02 | 10355 | 1.09 | 0.98 | 3.49 | 41.34 | 23.54 |
| Myoblasts C3 | 27.42 | 1787.04 | 10234 | 0.94 | 1.24 | 2.86 | 26.29 | 15.72 |
| Myoblasts SnC1 | 16.48 | 1198.37 | 3905 | 0.92 | 0.83 | 1.92 | 13.97 | 16.21 |
| Myoblasts SnC2 | 15.45 | 1052.99 | 27479 | 6.24 | 1.66 | 1.83 | 15.01 | 28.60 |
| Myoblasts SnC3 | 25.10 | 1887.97 | 22836 | 6.31 | 2.81 | 3.13 | 20.58 | 32.15 |
| OVCAR8 CM1 | 3.67 | 438.94 | 337 | 0.00 | 0.17 | 0.18 | 15.86 | 2.52 |
| OVCAR8 CM2 | 6.55 | 464.03 | 2517 | 0.54 | 0.17 | 0.59 | 21.99 | 4.15 |
| OVCAR8 CM3 | 3.89 | 408.46 | 434 | 0.00 | 0.15 | 0.32 | 19.07 | 2.98 |

Average concentration pg/ml/million cells

| | Activin A | ADAMTS13 | CCL3 | CCL4 | CCL5 | CCL17 | CCL22 | Fas |
|---|---|---|---|---|---|---|---|---|
| HUVEC Ctrl | 51.96 | 7875.73 | 86.59 | 149.10 | 3.84 | 87.62 | 31.21 | 21.17 |
| HUVEC SnC | 927.23 | 78558.99 | 624.06 | 1074.43 | 33.81 | 709.74 | 255.99 | 201.12 |
| Preadipocytes Ctrl | 150.04 | 5444.57 | 275.04 | 395.20 | 3.53 | 102.14 | 101.26 | 69.70 |
| Preadipocytes SnC | 4228.79 | 69551.29 | 345.31 | 1539.08 | 32.37 | 758.31 | 329.02 | 245.91 |
| Fibroblasts Ctrl | 51.16 | 5189.39 | 176.87 | 301.69 | 3.36 | 83.75 | 54.37 | 24.99 |
| Fibroblasts SnC | 223.52 | 13704.10 | 234.75 | 659.70 | 3.36 | 264.84 | 158.69 | 119.63 |
| Epithelial Ctrl | 71.61 | 10684.96 | 100.18 | 209.20 | 3.83 | 109.89 | 44.30 | 48.15 |
| Epithelial SnC | 269.98 | 42132.25 | 527.09 | 870.26 | 15.80 | 456.20 | 182.05 | 204.09 |
| Myoblasts Ctrl | 32.92 | 10535.49 | 243.66 | 299.51 | 1.83 | 128.10 | 66.92 | 29.73 |
| Myoblasts SnC | 83.68 | 8044.41 | 96.37 | 175.58 | 3.06 | 95.42 | 37.46 | 33.04 |
| OVCAR8 | 0.03 | 1010.17 | 59.83 | 40.51 | 0.39 | 16.70 | 10.85 | 3.31 |

Average concentration pg/ml/million cells

| | GDF15 | GDNF | ICAM1 | IL6 | IL7 | IL8 | IL15 | MMP2 |
|---|---|---|---|---|---|---|---|---|
| HUVEC Ctrl | 131.64 | 0.81 | 1663.08 | 13.29 | 3.70 | 25.77 | 3.32 | 5431.12 |
| HUVEC SnC | 5523.36 | 8.33 | 13775.73 | 88.61 | 30.27 | 1188.98 | 27.88 | 56017.01 |
| Preadipocytes Ctrl | 31.58 | 0.87 | 1293.45 | 24.03 | 2.90 | 5.20 | 0.18 | 4273.49 |
| Preadipocytes SnC | 217.83 | 10.16 | 15976.48 | 1897.60 | 33.08 | 76.31 | 24.72 | 23461.34 |
| Fibroblasts Ctrl | 18.16 | 0.58 | 2591.52 | 0.25 | 0.73 | 2.90 | 2.69 | 1629.66 |
| Fibroblasts SnC | 213.28 | 2.36 | 5345.50 | 13.92 | 7.98 | 35.78 | 4.87 | 7426.76 |
| Epithelial Ctrl | 7.27 | 1.06 | 2341.49 | 2.44 | 5.05 | 49.12 | 4.28 | 1733.65 |
| Epithelial SnC | 36.56 | 4.41 | 9889.50 | 9.37 | 20.96 | 177.48 | 17.48 | 13806.63 |
| Myoblasts Ctrl | 17.85 | 1.25 | 2583.20 | 0.39 | 4.69 | 3.04 | 3.36 | 5784.87 |
| Myoblasts SnC | 84.23 | 0.90 | 1855.87 | 1.51 | 3.69 | 6.05 | 2.83 | 10187.15 |
| OVCAR8 | 6.47 | 0.14 | 374.34 | 0.07 | 0.53 | 0.17 | 0.31 | 17.82 |

Average concentration pg/ml/million cells

| | MMP9 | OPN | PAI1 | PAI2 | SOST | TNFα | TNFR1 | VEGFA |
|---|---|---|---|---|---|---|---|---|
| HUVEC Ctrl | 18.42 | 2235.93 | 363998.62 | 11921.25 | 2.30 | 2.28 | 16.78 | 3.78 |
| HUVEC SnC | 152.52 | 58455.55 | 9882673.65 | 106493.25 | 21.86 | 18.63 | 190.46 | 34.28 |
| Preadipocytes Ctrl | 51.14 | 4571.96 | 5207.43 | 19.56 | 4.43 | 1.43 | 33.33 | 36.19 |
| Preadipocytes SnC | 156.59 | 12875.04 | 268070.41 | 34398.14 | 12.24 | 19.17 | 134.47 | 406.11 |
| Fibroblasts Ctrl | 37.96 | 4853.41 | 532.45 | 18.64 | 2.38 | 0.23 | 27.75 | 27.16 |
| Fibroblasts SnC | 68.09 | 6727.63 | 5332.99 | 18.64 | 2.38 | 4.53 | 83.75 | 64.45 |
| Epithelial Ctrl | 25.40 | 2755.52 | 80571.36 | 12380.25 | 2.81 | 2.82 | 18.32 | 75.85 |
| Epithelial SnC | 104.83 | 13738.53 | 306074.80 | 49342.78 | 10.76 | 12.19 | 93.38 | 928.57 |
| Myoblasts Ctrl | 29.90 | 2253.46 | 7757.16 | 679.22 | 1.02 | 2.83 | 33.26 | 17.42 |
| Myoblasts SnC | 19.01 | 1379.78 | 18073.13 | 4488.96 | 1.77 | 2.29 | 16.52 | 25.65 |
| OVCAR8 | 4.70 | 437.14 | 1095.95 | 181.35 | 0.16 | 0.36 | 18.97 | 3.22 |

Q value

| | Activin A | ADAMTS13 | CCL3 | CCL4 | CCL5 | CCL17 | CCL22 | Fas |
|---|---|---|---|---|---|---|---|---|
| HUVEC | 0.00024171 | 0.000241708 | 0.00010788 | 0.0002417 | 0.000242 | 0.00024 | 0.000242 | 0.000423 |
| Preadipocytes | 0.0010461 | 1.74981E-05 | 0.12872198 | 0.0002243 | 0.000425 | 0.00015 | 0.000491 | 0.000224 |
| Fibroblasts | 0.10379919 | 0.258198834 | 0.22001861 | 0.0410509 | 0.37764 | 0.1038 | 0.03253 | 0.077624 |
| Epithelial | 0.04336563 | 0.012571996 | 6.0446E-05 | 0.012572 | 0.012572 | 0.01257 | 0.012572 | 0.015722 |
| Myoblasts | 0.37056562 | 0.370565617 | 0.00778454 | 0.2701084 | 0.370566 | 0.37057 | 0.270108 | 0.65362 |

Q value

| | GDF15 | GDNF | ICAM1 | IL6 | IL7 | IL8 | IL15 | MMP2 |
|---|---|---|---|---|---|---|---|---|
| HUVEC | 0.00024171 | 0.000964511 | 0.00032401 | 0.0002417 | 0.000242 | 0.00032 | 0.000242 | 0.000242 |
| Preadipocytes | 0.00233847 | 0.000491411 | 0.00018804 | 0.0006728 | 0.000224 | 0.0122 | 0.000237 | 0.000975 |
| Fibroblasts | 0.13764942 | 0.103799194 | 0.12539468 | 0.0462085 | 0.127534 | 0.24071 | 0.263376 | 0.103799 |
| Epithelial | 0.012572 | 0.01572196 | 0.012572 | 0.0385087 | 0.015722 | 0.04832 | 0.012572 | 0.015722 |
| Myoblasts | 0.05105334 | 0.370565617 | 0.37056562 | 0.2874725 | 0.448423 | 0.37057 | 0.650236 | 0.370566 |

Q value

| | MMP9 | OPN | PAI1 | PAI2 | SOST | TNFα | TNFR1 | VEGFA |
|---|---|---|---|---|---|---|---|---|
| HUVEC | 0.00034986 | 0.000241708 | 0.00028427 | 4.444E-05 | 0.000257 | 0.00024 | 0.000242 | 0.000242 |
| Preadipocytes | 0.00049141 | 0.000491411 | 0.01778193 | 0.0295985 | 0.015649 | 0.00049 | 0.000491 | 0.003759 |
| Fibroblasts | 0.09813872 | 0.242602299 | 0.0029603 | | 0.37764 | 0.13834 | 0.010563 | 0.350638 |
| Epithelial | 0.012572 | 0.012571996 | 0.04275467 | 0.0295985 | 0.030531 | 0.01691 | 0.012572 | 0.012572 |
| Myoblasts | 0.27010838 | 0.27010838 | 0.15055627 | 0.0789167 | 0.370566 | 0.45949 | 0.270108 | 0.370566 |

1-20. (canceled)

21. A method for treating a mammal having frailty, wherein said method comprises:
(a) identifying said mammal as having an elevated level of expression for each of four or more SASP polypeptides, for said mammal’s chronological age, in a sample from said mammal; and
(b) administering a senotherapeutic agent to said mammal.

22. (canceled)

23. The method of claim 21, wherein said mammal is a human.

24. The method of claim 21, wherein said senotherapeutic agent is selected from the group consisting of dasatinib, quercetin, navitoclax, A1331852, A1155463, fisetin, luteolin, geldanamycin, tanespimycin, alvespimycin, piperlongumine, panobinostat, FOX04-related peptides, nutlin3a, ruxolitinib, metformin, and rapamycin.

25. The method of claim 21, wherein said senotherapeutic agent is effective to reduce or eliminate a symptom of frailty.

26. The method of claim 25, wherein said symptom of frailty is selected from the group consisting of unintentional weight loss, exhaustion, muscle weakness, slowness while walking, low levels of activity, inflammation, difficulties with activities of daily living, and combinations thereof.

27. A method for improving the outcome of a mammal undergoing a medical intervention, wherein said method comprises:
(a) identifying said mammal as having an elevated level of expression for each of four or more SASP polypeptides, for said mammal’s chronological age, in a sample from said mammal; and
(b) administering a senotherapeutic agent to said mammal.

28. (canceled)

29. The method of claim 27, wherein said mammal is a human.

30. The method of claim 27, wherein said senotherapeutic agent is selected from the group consisting of dasatinib, quercetin, navitoclax, A1331852, A1155463, fisetin, luteolin, geldanamycin, tanespimycin, alvespimycin, piperlongumine, panobinostat, FOX04-related peptides, nutlin3a, ruxolitinib, metformin, and rapamycin.

31. The method of claim 27, wherein said senotherapeutic agent is effective to reduce or eliminate an adverse event that can occur following a medical intervention.

32. The method of claim 31, wherein said adverse event is selected from the group consisting of myocardial infarction, new arrhythmia, new conduction abnormality, stroke, deep venous thrombosis, pulmonary emboli, pneumonia, plural effusion, new renal insufficiency, GI bleeding, new seizure disorder, significant hypotension, significant tachycardia, significant bradycardia, urinary tract infection, other infection, acute dementia, vascular complication, acute kidney injury, and combinations thereof.

33. The method of claim 27, wherein said medical intervention comprises a surgery.

34. A method for reducing a systemic senescent cell burden of a mammal, wherein said method comprises:
(a) identifying said mammal as having an elevated level of expression for each of four or more SASP polypeptides, for said mammal’s chronological age, in a sample from said mammal; and
(b) administering a senotherapeutic agent to said mammal.

35. (canceled)

36. The method of claim 34, wherein said mammal is a human.

37. The method of claim 34, wherein said senotherapeutic agent is selected from the group consisting of dasatinib, quercetin, navitoclax, A1331852, A1155463, fisetin, luteolin, geldanamycin, tanespimycin, alvespimycin, piperlongumine, panobinostat, FOX04-related peptides, nutlin3a, ruxolitinib, metformin, and rapamycin.

38-52. (canceled)


SEQUENCE LISTING (3 sequences)

SEQ ID NO: 1 - DNA, 21 bp, artificial
  synthetic oligonucleotide primer
ccaacgcaccgaatagttacg

SEQ ID NO: 2 - DNA, 18 bp, artificial
  synthetic oligonucleotide primer
gcgctgcccatcatcatg

SEQ ID NO: 3 - DNA, 18 bp, artificial
  synthetic oligonucleotide probe
cctggatcggcctccgac